(19) United States
(12) Patent Application Publication
(10) Pub. No.: US 2024/0374524 A1
(43) Pub. Date: Nov. 14, 2024

(54) INCREASING PERMEATION FOR PRE-GASTRIC ABSORPTION OF ACTIVE PHARMACEUTICAL INGREDIENTS

(71) Applicant: Catalent U.K. Swindon Zydis Limited, Glasgow (GB)

(72) Inventors: Ruba BNYAN, Twickenham (GB); Afzal Ur Rahman MOHAMMED, Birmingham (GB); Daniel J. KIRBY, Tamworth (GB); Yik Teng WONG, Wiltshire (GB)

(73) Assignee: Catalent U.K. Swindon Zydis Limited, Glasgow (GB)

(21) Appl. No.: 18/660,567

(22) Filed: May 10, 2024

Related U.S. Application Data
(60) Provisional application No. 63/465,578, filed on May 11, 2023.

(51) Int. Cl. A61K 9/20; A61K 31/403; A61K 31/426; A61K 31/5415

(52) U.S. Cl. CPC A61K 9/2013; A61K 9/2063; A61K 9/2095; A61K 31/403; A61K 31/426; A61K 31/5415

(57) ABSTRACT

Provided are pharmaceutical compositions and methods for preparing pharmaceutical compositions comprising permeation enhancers for pre-gastric absorption of the active pharmaceutical ingredient (API). Specifically, the permeation enhancers can be chosen based on the specific biopharmaceutics classification system (BCS) API classification and subclassification.

### REFERENCE TO RELATED APPLICATIONS

This application claims the benefit of U.S. Provisional Application No. 63/465,578, filed May 11, 2023, the entire contents of which are incorporated herein by reference.

### FIELD OF THE INVENTION

This disclosure relates to formulations and compositions and methods of making thereof that include permeation enhancers for increasing permeation of active pharmaceutical ingredients (APIs). More specifically, this disclosure relates to orally disintegrating dosage forms and methods of making thereof that include specific permeation enhancers for a given API to aid pre-gastric absorption of the API.

### BACKGROUND

A majority of drugs are absorbed by the gastrointestinal system. Some drugs may be absorbed prior to entering the gastrointestinal system through buccal, sublingual, pharyngeal, and esophageal routes. For some drug products, using pre-gastric absorption routes is a preferred option because it can allow the drug molecules to diffuse directly through mucosal tissues and into the circulatory system. As a result, the drug can have a quicker onset and/or avoid being metabolized by the liver.

### SUMMARY

Drug development for pre-gastric delivery with freeze-dried tablets can be challenging when an active pharmaceutical ingredient (API) has low solubility and/or permeability. Absorption of an API can be strongly influenced by the physicochemical properties of the API such as molecular weight, solubility, hydrophilicity, and lipophilicity, as well as by factors such as drug loading, mucosal contact time, the drug formulation, and excipients used. APIs with poor solubility and/or permeability characteristics can give rise to poor absorption and variable drug bioavailability.

Various formulation approaches have been used to improve the absorption of drugs with poor permeability, including the addition of permeation enhancers (PEs) also known as permeation agents (PAs). In particular, PEs can be added to drug formulations to enhance permeability of APIs in biopharmaceutics classification system (BCS) classes II and III. The BCS system classifies APIs according to their solubility and permeability characteristics, where BCS Class II APIs are characterized by low solubility and high permeability, and BCS Class III APIs are characterized by high solubility and low permeability. Although some PEs can help Class II and III APIs overcome the oral mucosa barrier, it is difficult to predict which PEs will work with a given API. The selection of appropriate PEs for each type of API is largely based on empirical, trial-and-error experience, and most work on PEs to date has focused on improving drug permeation of intestinal and dermal tissues. The effect of PEs on pre-gastric absorption through the oral mucosal tissue, especially in a freeze-dried orally dissolvable tablet (ODT) format, is less established. As a result, formulations are often unsuccessful, and the required reformulation activities increase drug development time and incur additional development costs.

Disclosed herein are pharmaceutical formulations and compositions and methods of making thereof utilizing appropriate PEs for a given Class II and/or Class III API.

In some embodiments, a pharmaceutical composition includes a pharmaceutically effective amount of a biopharmaceutics classification system (BCS) Class II active pharmaceutical ingredient (API) or pharmaceutically acceptable salt or solvate thereof having a solubility of less than 0.1 mg/mL and a log P value greater than 2.5; a permeation enhancer comprising at least one selected from the group of counterions, a pH modifier, a surfactant with a hydrophilic lipophilic balance (HLB) greater than 10, a bile salt, a micelle, or a fatty acid; a matrix former; and a structure former. In some embodiments, the permeation enhancer comprises a surfactant with a hydrophilic lipophilic balance (HLB) greater than 10. In some embodiments, a molar ratio of API to the surfactant is 5:1 to 1:5 and/or the pharmaceutical composition comprises 0.5-20 wt. % the surfactant. In some embodiments, the pharmaceutical composition includes a pH modifier. In some embodiments, the permeation enhancer comprises a bile salt. In some embodiments, a molar ratio of API to the bile salt is 15:1 to 1:15 and/or the pharmaceutical composition comprises 1-20 wt. % the bile salt. In some embodiments, the permeation enhancer comprises a fatty acid. In some embodiments, a molar ratio of API to fatty acid is 3:1 to 1:3 and/or the pharmaceutical composition comprises 0.25-5 wt. % fatty acid. In some embodiments, the permeation enhancer comprises counterions. In some embodiments, a molar ratio of API to counterions is 6:1 to 1:6 and/or the pharmaceutical composition comprises 0.1-25 wt. % counterions. In some embodiments, the pharmaceutical composition includes a pH modifier. In some embodiments, the pharmaceutical composition includes 25-60 wt. % matrix former. In some embodiments, the matrix former comprises gelatin, pullulan, starch, or combinations thereof. In some embodiments, the gelatin comprises fish gelatin, bovine gelatin, porcine gelatin, or combination thereof. In some embodiments, the gelatin is fish gelatin and the fish gelatin is high molecular weight fish gelatin. In some embodiments, the pharmaceutical composition includes 20-45 wt. % structure former. In some embodiments, the structure former comprises mannitol. In some embodiments, the pharmaceutical composition comprises 1-35 wt. % the BCS Class II API or pharmaceutically acceptable salt or solvate thereof.

In some embodiments, a pharmaceutical composition includes: a pharmaceutically effective amount of a biopharmaceutics classification system (BCS) Class II active pharmaceutical ingredient (API) or pharmaceutically acceptable salt or solvate thereof having a solubility of 0.1-1 mg/mL and a log P value of 1-2.5; a permeation enhancer comprising at least one selected from the group of counterions, a pH modifier, a surfactant with a hydrophilic lipophilic balance (HLB) greater than 10, a bile salt, or a micelle; a matrix former; and a structure former. In some embodiments, the permeation enhancer comprises a surfactant with a hydrophilic lipophilic balance (HLB) greater than 10. In some embodiments, a molar ratio of API to surfactant is 5:1 to 1:5 and/or the pharmaceutical composition comprises 0.5-20 wt. % surfactant. In some embodiments, the permeation enhancer comprises a bile salt. In some embodiments, a molar ratio of API to bile salt is 15:1 to 1:15 and/or the pharmaceutical composition comprises 1-20 wt. % bile salt. In some embodiments, the permeation enhancer comprises counterions. In some embodiments, a molar ratio of API to counterions is 6:1 to 1:6 and/or the pharmaceutical composition comprises 0.1-25 wt. % counterions. In some embodiments, the pharmaceutical composition includes a pH modifier. In some embodiments, the pharmaceutical composition includes 1-35 wt. % the pharmaceutically effective amount of a biopharmaceutics classification system (BCS) Class II active pharmaceutical ingredient (API) or pharmaceutically acceptable salt or solvate thereof having a solubility of 0.1-1 mg/mL and a log P value of 1-2.5. In some embodiments, the pharmaceutical composition includes 25-60 wt. % matrix former. In some embodiments, the matrix former comprises gelatin, pullulan, starch, or combinations thereof. In some embodiments, the gelatin comprises fish gelatin, bovine gelatin, porcine gelatin, or combination thereof. In some embodiments, the gelatin is fish gelatin and the fish gelatin is high molecular weight fish gelatin. In some embodiments, the pharmaceutical composition includes 20-45 wt. % structure former. In some embodiments, the structure former comprises mannitol.

In some embodiments, a pharmaceutical composition includes: a pharmaceutically effective amount of a biopharmaceutics classification system (BCS) Class III active pharmaceutical ingredient (API) or pharmaceutically acceptable salt or solvate thereof having a solubility of 1-10 mg/mL and a log P value of less than 1; a permeation enhancer comprising at least one selected from the group of counterions, a pH modifier, or a bile salt; a matrix former; and a structure former. In some embodiments, the permeation enhancer comprises a bile salt. In some embodiments, a molar ratio of API to bile salt is 15:1 to 1:15 and/or the pharmaceutical composition comprises 1-20 wt. % bile salt. In some embodiments, the permeation enhancer comprises counterions. In some embodiments, a molar ratio of API to counterions is 6:1 to 1:6 and/or the pharmaceutical composition comprises 0.1-25 wt. % counterions. In some embodiments, the pharmaceutical composition includes a pH modifier. In some embodiments, a pharmaceutical composition includes 25-60 wt. % matrix former. In some embodiments, the matrix former comprises gelatin, pullulan, starch, or combinations thereof. In some embodiments, the gelatin comprises fish gelatin, bovine gelatin, porcine gelatin, or combination thereof. In some embodiments, the gelatin is fish gelatin and the fish gelatin is high molecular weight fish gelatin. In some embodiments, the pharmaceutical composition includes 20-45 wt. % structure former. In some embodiments, the structure former comprises mannitol. In some embodiments, the pharmaceutical composition includes 1-35 wt. % the BCS Class III API or pharmaceutically acceptable salt or solvate thereof.

In some embodiments, a pharmaceutical composition includes a pharmaceutically effective amount of a biopharmaceutics classification system (BCS) Class III active pharmaceutical ingredient (API) or pharmaceutically acceptable salt or solvate thereof having a solubility of 10-33 mg/mL and a log P value of less than 1; a permeation enhancer comprising at least one selected from the group of counterions or a pH modifier; a matrix former; and a structure former. In some embodiments, the permeation enhancer comprises counterions. In some embodiments, a molar ratio of API to counterions is 6:1 to 1:6 and/or the pharmaceutical composition comprises 0.1-25 wt. % counterions. In some embodiments, the pharmaceutical composition includes 25-60 wt. % matrix former. In some embodiments, the matrix former comprises gelatin, pullulan, starch, or combinations thereof. In some embodiments, the gelatin comprises fish gelatin, bovine gelatin, porcine gelatin, or combination thereof. In some embodiments, the gelatin is fish gelatin and the fish gelatin is high molecular weight fish gelatin. In some embodiments, the pharmaceutical compositions include 20-45 wt. % structure former. In some embodiments, the structure former comprises mannitol. In some embodiments, the pharmaceutical composition includes 1-35 wt. % the pharmaceutically effective amount of a biopharmaceutics classification system (BCS) Class II active pharmaceutical ingredient (API) or pharmaceutically acceptable salt or solvate thereof having a solubility of 10-33 mg/mL and a log P value of less than 1.

In some embodiments, the pharmaceutical composition is a solid dosage form. In some embodiments, a method of treating a patient includes placing the solid dosage form in an oral cavity of a person in need of the treatment. In some embodiments, the placement in the oral cavity is placement on or under the tongue or in the buccal or pharyngeal region. In some embodiments, a method of forming a solid dosage form includes: dosing

a pharmaceutical formulation into a preformed mold, wherein the pharmaceutical formulation comprises: a pharmaceutically effective amount of a biopharmaceutics classification system (BCS) Class II active pharmaceutical ingredient (API) or pharmaceutically acceptable salt or solvate thereof having a solubility of less than 0.1 mg/mL and a log P value greater than 2.5; a permeation enhancer comprising at least one selected from the group of counterions, a pH modifier, a surfactant with a hydrophilic lipophilic balance (HLB) greater than 10, a bile salt, a micelle, or a fatty acid; 1-10 wt. % matrix former; and 1-10 wt. % of a structure former; freezing the dosed pharmaceutical formulation; and freeze-drying the frozen pharmaceutical formulation to form the dosage form. In some embodiments, the permeation enhancer comprises a surfactant with a hydrophilic lipophilic balance greater than 10. In some embodiments, the pharmaceutical formulation comprises 0.01-5 wt. % the surfactant. In some embodiments, the surfactant has a concentration of 0.1-30× its critical micellar concentrations (CMC) in the pharmaceutical formulation. In some embodiments, the surfactant has a concentration of 0.5-3×CMC in the pharmaceutical formulation. In some embodiments, the pharmaceutical formulation has a molar ratio of API to the surfactant of 5:1 to 1:5. In some embodiments, the permeation enhancer comprises a bile salt. In some embodiments, the pharmaceutical formulation comprises 0.25-5 wt. % bile salt. In some embodiments, the bile salt has a concentration of 0.5-5×CMC in the pharmaceutical formulation. In some embodiments, the pharmaceutical formulation has a molar ratio of API to the bile salt of 15:1 to 1:15. In some embodiments, the molar ratio is 5:1 to 1:5. In some embodiments, the permeation enhancer comprises a fatty acid. In some embodiments, the pharmaceutical formulation comprises 0.03-0.15 wt. % fatty acid. In some embodiments, the pharmaceutical formulation has a molar ratio of API to fatty acid of 3:1 to 1:3. In some embodiments, the permeation enhancer comprises counterions. In some embodiments, the pharmaceutical formulation comprises 0.025-5 wt. % counterions. In some embodiments, the pharmaceutical formulation has a molar ratio of API to counterions of 6:1 to 1:6. In some embodiments, the molar ratio is 3:1 to 1:3. In some embodiments, the pharmaceutical formulation comprises a pH modifier. In some embodiments, the pharmaceutical formulation comprises 0.1-5 wt. % the BCS Class II API or pharmaceutically acceptable salt or solvate thereof.

In some embodiments, a method of forming a solid dosage form includes: dosing a pharmaceutical formulation into a preformed mold, wherein the pharmaceutical formulation comprises: a pharmaceutically effective amount of a biopharmaceutics classification system (BCS) Class II active pharmaceutical ingredient (API) or pharmaceutically acceptable salt or solvate thereof having a solubility of 0.1-1 mg/mL and a log P value 1-2.5; a permeation enhancer comprising at least one selected from the group of counterions, a pH modifier, a surfactant with a hydrophilic lipophilic balance (HLB) greater than 10, a bile salt, or a micelles; 1-10 wt. % matrix former; and 1-10 wt. % of a structure former; freezing the dosed pharmaceutical formulation; and freeze-drying the frozen pharmaceutical formulation to form the dosage form. In some embodiments, the permeation enhancer comprises a surfactant with a hydrophilic lipophilic balance greater than 10. In some embodiments, the pharmaceutical formulation comprises 0.01-5 wt. % the surfactant. In some embodiments, the surfactant has a concentration 0.1-30× its critical micellar concentrations (CMC) of in the pharmaceutical formulation. In some embodiments, the surfactant has a concentration of 0.5-3×CMC in the pharmaceutical formulation. In some embodiments, the pharmaceutical formulation has a molar ratio of API to the surfactant of 5:1 to 1:5. In some embodiments, the permeation enhancer comprises a bile salt. In some embodiments, the pharmaceutical formulation comprises 0.25-5 wt. % bile salt. In some embodiments, the bile salt has a concentration of 0.5-5×CMC in the pharmaceutical formulation. In some embodiments, the pharmaceutical formulation has a molar ratio of API to the bile salt of 15:1 to 1:15. In some embodiments, the molar ratio is 5:1 to 1:5. In some embodiments, the permeation enhancer comprises counterions. In some embodiments, the pharmaceutical formulation comprises 0.025-5 wt. % counterions. In some embodiments, the pharmaceutical formulation has a molar ratio of API to counterions of 6:1 to 1:6. In some embodiments, the molar ratio is 3:1 to 1:3. In some embodiments, the pharmaceutical formulation comprises a pH modifier. In some embodiments, the pharmaceutical formulation comprises 0.1-5 wt. % BCS Class II API or pharmaceutically acceptable salt or solvate thereof.

In some embodiments, a method of forming a solid dosage form includes: dosing a pharmaceutical formulation into a preformed mold, wherein the pharmaceutical formulation comprises: a pharmaceutically effective amount of a biopharmaceutics classification system (BCS) Class III active pharmaceutical ingredient (API) or pharmaceutically acceptable salt or solvate thereof having a solubility of 1-10 mg/mL and a log P value less than 1; a permeation enhancer comprising at least one selected from the group of counterions, a pH modifier, or a bile salt; 1-10 wt. % matrix former; and 1-10 wt. % of a structure former; freezing the dosed pharmaceutical formulation; and freeze-drying the frozen pharmaceutical formulation to form the dosage form. In some embodiments, the permeation enhancer comprises a bile salt. In some embodiments, the pharmaceutical formulation comprises 0.25-5 wt. % bile salt. In some embodiments, the bile salt has a concentration of 0.5-5×CMC in the pharmaceutical formulation. In some embodiments, the pharmaceutical formulation has a molar ratio of API to the bile salt of 15:1 to 1:15. In some embodiments, the molar ratio is 5:1 to 1:5. In some embodiments, the permeation enhancer comprises counterions. In some embodiments, the pharmaceutical formulation comprises 0.025-5 wt. % counterions. In some embodiments, the pharmaceutical formulation has a molar ratio of API to counterions of 6:1 to 1:6. In some embodiments, the pharmaceutical formulation comprises a pH modifier. In some embodiments, the molar ratio is 3:1 to 1:3. In some embodiments, the pharmaceutical formulation comprises 0.1-5 wt. % BCS Class III API or pharmaceutically acceptable salt or solvate thereof.

In some embodiments, a method of forming a solid dosage form includes: dosing a pharmaceutical formulation into a preformed mold, wherein the pharmaceutical formulation comprises: a pharmaceutically effective amount of a biopharmaceutics classification system (BCS) Class III active pharmaceutical ingredient (API) or pharmaceutically acceptable salt or solvate thereof having a solubility of 10-33 mg/mL and a log P value less than 1; a permeation enhancer comprising at least one selected from the group of counterions or a pH modifier, 1-10 wt. % matrix former; and 1-10 wt. % of a structure former; freezing the dosed pharmaceutical formulation; and freeze-drying the frozen pharmaceutical formulation to form the dosage form. In some embodiments, the permeation enhancer comprises counterions. In some embodiments, the pharmaceutical formulation comprises 0.025-5 wt. % counterions. In some embodiments, the pharmaceutical formulation has a molar ratio of API to counterions of 6:1 to 1:6. In some embodiments, the molar ratio is 3:1 to 1:3. In some embodiments, the pharmaceutical formulation comprises a pH modifier. In some embodiments, the pharmaceutical formulation comprises 0.1-5 wt. % BCS Class III API or pharmaceutically acceptable salt or solvate thereof. In some embodiments, the matrix former comprises gelatin, pullulan, starch, or combinations thereof. In some embodiments, the gelatin comprises fish gelatin, bovine gelatin, porcine gelatin, or combination thereof. In some embodiments, the gelatin is fish gelatin and the fish gelatin is high molecular weight fish gelatin. In some embodiments, the structure former comprises mannitol.

In some embodiments, a pharmaceutical composition includes: a

pharmaceutically effective amount of a biopharmaceutics classification system (BCS) Class II active pharmaceutical ingredient (API) or pharmaceutically acceptable salt or solvate thereof having a solubility of 0.1-1 mg/mL and a log P value of 1-2.5; a permeation inhibitor r comprising at least one selected from the group of a surfactant with a hydrophilic lipophilic balance (HLB) less than 10 or a fatty acid; a matrix former; and a structure former. In some embodiments, the permeation inhibitor comprises a fatty acid. In some embodiments, the pharmaceutical formulation comprises 0.25-0.5 wt. % fatty acid. In some embodiments, the pharmaceutical formulation has a molar ratio of API to fatty acid of 3:1 to 1:3. In some embodiments, the permeation inhibitor comprises the surfactant with a HLB less than 10. In some embodiments, a molar ratio of API to surfactant is 5:1 to 1:5.

Additional advantages will be readily apparent to those skilled in the art from the following detailed description. The examples and descriptions herein are to be regarded as illustrative in nature and not restrictive.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### BRIEF DESCRIPTION OF THE FIGURES

FIG. 1A shows various pharmaceutical pre-formulations (binary mixture of API and permeation enhancer) and piroxicam pharmaceutical compositions (ODTs) tested for permeability herein when surfactants are used as permeation agents.

FIG. 1B shows various pharmaceutical pre-formulations (binary mixture of API and permeation enhancer) and piroxicam pharmaceutical compositions (ODTs) tested for permeability herein when bile salts are used as permeation agents.

FIG. 1C shows various pharmaceutical pre-formulations (binary mixture of API and permeation enhancer) tested for permeability herein when fatty acids are used as permeation agents.

FIG. 1D shows various pharmaceutical pre-formulations (binary mixture of API and permeation enhancer) tested for permeability herein when pH modifiers are used as permeation agents.

FIG. 1E shows various pharmaceutical pre-formulations (binary mixture of API and permeation enhancer) tested for permeability herein when counterions are used as permeation agents.

FIG. 2A shows the binary mixes of atenolol and various permeation agents tested for permeability herein.

FIG. 2B shows the binary mixes of carvedilol and various permeation agents tested for permeability herein.

FIG. 2C shows the binary mixes of famotidine and various permeation agents tested for permeability herein.

FIG. 2D shows the binary mixes of piroxicam and various permeation agents and the orally disintegrating tablets containing piroxicam and various permeation agents tested for permeability herein.

FIG. 3 depicts an empirically derived example set of permeation enhancers associated with various combinations of API BCS information and sub-classification information.

FIG. 4 illustrates physicochemical properties of various Class II and Class III APIs tested herein.

FIG. 5 is exemplary permeability data illustrating the effect of using polymeric non-ionic surfactants as permeation enhancers for Piroxicam.

FIG. 6 is exemplary permeability data illustrating the effect of using various non-ionic and ionic surfactants and fatty acids as permeability enhancers for Piroxicam.

FIG. 7 is exemplary permeability data illustrating the effect of using bile salts as permeability enhancers for Piroxicam.

FIG. 8 is exemplary permeability data illustrating the effect of using various counterions as permeability enhancers for Piroxicam.

FIG. 9 is exemplary permeability data illustrating the effect of using a polymeric non-ionic surfactant as a permeability enhancer for Piroxicam in a freeze-dried orally disintegrating tablet.

FIG. 10 is exemplary permeability data illustrating the effect of using bile salts and anionic surfactants as permeability enhancers for Piroxicam in a freeze-dried orally disintegrating tablet.

FIG. 11 is exemplary permeability data illustrating the effect of using a pH modifier as a permeability enhancer for Carvedilol.

FIG. 12 is exemplary permeability data illustrating the effect of using non-ionic surfactants and a combination of non-ionic surfactants and pH modifiers as permeability enhancers for Carvedilol.

FIG. 13 is exemplary permeability data illustrating the effect of using various ionic and non-ionic surfactants and fatty acids as permeability enhancers for Carvedilol.

FIG. 14 is exemplary permeability data illustrating the effect of using bile salts and a combination of bile salts and pH modifiers as permeability enhancers for Carvedilol.

FIG. 15 is exemplary permeability data illustrating the effect of using various counterions as permeability enhancers for Carvedilol.

FIG. 16 is exemplary permeability data illustrating the effect of using bile salts and non-ionic surfactants as permeability enhancers for Famotidine.

FIG. 17 is exemplary permeability data illustrating the effect of using various counterions as permeability enhancers for Famotidine.

FIG. 18 is exemplary permeability data illustrating the effect of using various ionic and non-ionic surfactants and bile salts as permeability enhancers for Atenolol.

FIG. 19 is exemplary permeability data illustrating the effect of using PH modifiers as permeability enhancers for Atenolol.

FIG. 20 is exemplary permeability data illustrating the effect of using various counterions as permeability enhancers for Atenolol.

FIG. 21 is a flowchart depicting an empirically derived decision-making model for selecting suitable permeation enhancers based on various combinations of API BCS information and sub-classification information.

FIG. 22A illustrates exemplary permeability data illustrating that the base matrix excipients have no effect on the permeability of carvedilol.

FIG. 22B illustrates exemplary permeability data illustrating that the base matrix excipients have no effect on permeability of carvedilol versus a positive effect with a non-ionic surfactant (PF127).

FIG. 22C illustrate exemplary permeability data illustrating that base matrix excipients have no effect on permeability of piroxicam (at low dose).

FIG. 22D illustrates exemplary permeability data illustrating that base matrix (with excipients) has no effect on permeability of piroxicam (at low dose) versus a positive effect of a non-ionic surfactant (PF127).

FIG. 22E illustrates exemplary permeability data illustrating that base matrix excipients have no effect on permeability of piroxicam (at high dose).

FIG. 22F illustrates exemplary permeability data illustrating that base matrix excipients have no effect on permeability of piroxicam (at high dose) versus a positive effect of a non-ionic surfactant (PF127).

FIG. 22G illustrates exemplary permeability data illustrating that base matrix excipients have no enhancement effect on the permeability of atenolol.

### DETAILED DESCRIPTION

Described herein are exemplary embodiments of pharmaceutical formulations and compositions and methods of making thereof utilizing appropriate PEs for a given Class II and/or Class III API. Specifically, as explained in detail in the Examples herein, Applicant has discovered suitable permeation enhancers for aiding pre-gastric absorption for a given active pharmaceutical ingredient.

The combination of permeation enhancer and API can then be incorporated into a pharmaceutical composition (e.g., dosage form). In some embodiments, the pharmaceutical compositions including the permeation enhancer for the given APIs can be produced by a freeze-drying process.

In some embodiments, the API and permeation enhancer can be added to a base matrix to form a pharmaceutical formulation. As used herein, the pharmaceutical formulation refers to the pharmaceutical formulation prior to lyophilization and pharmaceutical composition refers to the composition post-lyophilization. The base matrix can help provide the structure for the final dosage form. In some embodiments, the base matrix can include at least a matrix former and a structure former. Examples of matrix formers and structure formers can be found in U.S. Pat. Nos. 6,316,027; 6,413,549; and 6,509,040, all of which are hereby incorporated by reference in their entirety.

The matrix former can provide the network structure that imparts strength and resilience to the dosage form. In some embodiments, the matrix former can be gelatin, pullulan, starch, hydrolyzed dextran, dextrin, alginates, polyvinyl alcohol, polyvinylpyrrolidone, acacia, cellulosic polymers (e.g., hydropropylmethyl cellulose, hydroxyethyl methyl cellulose, hydroxypropyl cellulose, and/or methylcellulose), soy, wheat, or combinations thereof. In some embodiments, the gelatin can be fish gelatin, bovine gelatin, porcine gelatin, or combination thereof. In some embodiments, the fish gelatin can be high molecular weight fish gelatin. In some embodiments, the fish gelatin can be high molecular weight fish gelatin, standard molecular weight fish gelatin, or combinations thereof. High molecular weight fish gelatin is defined as a fish gelatin in which more than 50% of the molecular weight distribution is greater than 30,000 Daltons. Standard molecular weight fish gelatin is defined as fish gelatin in which more than 50% of the molecular weight distribution is below 30,000 Daltons.

In some embodiments, the pharmaceutical formulation (pre-freeze drying) can include at least about 1 wt. %, at least about 2 wt. %, at least about 3 wt. %, at least about 4 wt. %, at least about 5 wt. %, at least about 6 wt. %, at least about 7 wt. %, or at least about 8 wt. % matrix former. In some embodiments, the pharmaceutical formulation can include at most about 15 wt. %, at most about 12 wt. %, at most about 10 wt. %, at most about 9 wt. %, at most about 8 wt. %, at most about 7 wt. %, at most about 6 wt. %, or at most about 5 wt. % matrix former. In some embodiments, the pharmaceutical formulation can include about 1-15 wt. %, about 1-10 wt. %, about 3-7 wt. %, about 4-6 wt. %, or about 5 wt. % matrix former.

The structure former can provide structural robustness to the dosage form. In some embodiments, the structure former can include sugars, including but not limited to mannitol, dextrose, lactose, galactose, cyclodextrin, or some combination thereof. In some embodiments, the pharmaceutical formulation (pre-freeze drying) can include at least about 1 wt. %, at least about 2 wt. %, at least about 3 wt. %, at least about 4 wt. %, at least about 5 wt. %, at least about 6 wt. %, at least about 7 wt. %, or at least about 8 wt. % structure former. In some embodiments, the pharmaceutical formulation can include at most about 15 wt. %, at most about 12 wt. %, at most about 10 wt. %, at most about 9 wt. %, at most about 8 wt. %, at most about 7 wt. %, at most about 6 wt. %, or at most about 5 wt. % structure former. In some embodiments, the pharmaceutical formulation can include about 1-15 wt. %, about 1-10 wt. %, about 2-6 wt. %, about 3-5 wt. %, or about 4 wt. % structure former.

In some embodiments, the base matrix (and thus the pharmaceutical formulation) can include pharmaceutically acceptable agents or excipients. Such additional pharmaceutically acceptable agents or excipients include, without limitation, sugars, such as mannitol, dextrose, and lactose, inorganic salts, such as sodium chloride and aluminum silicates, gelatins of mammalian origin, fish gelatin, modified starches, preservatives, antioxidants, surfactants, viscosity enhancers, coloring agents, flavoring agents, pH modifiers, sweeteners, taste-masking agents, and combinations thereof. Suitable coloring agents can include red, black and yellow iron oxides and FD & C dyes such as FD & C Blue No. 2 and FD & C Red No. 40, and combinations thereof. Suitable flavoring agents can include mint, raspberry, licorice, orange, lemon, grapefruit, caramel, vanilla, cherry and grape flavors and combinations of these. Suitable pH modifiers can include citric acid, tartaric acid, phosphoric acid, hydrochloric acid, maleic acid, sodium hydroxide (e.g., 3% w/w sodium hydroxide solution), and combinations thereof. Suitable sweeteners can include, sucralose aspartame, acesulfame K and thaumatin, and combinations thereof. Suitable taste-masking agents can include a range of flavorings and combinations thereof. One of ordinary skill in the art can readily determine suitable amounts of these various additional excipients if desired.

In addition, the base matrix (and thus the pharmaceutical formulation) can include a solvent. In some embodiments, the solvent can be water (e.g., purified water). In some embodiments, the pharmaceutical formulation includes an amount of solvent such that the pharmaceutical formulation is q.s. 100%.

In some embodiments, the base matrix can be prepared by dissolving the matrix former and structural former in solvent to form a pre-mix. In some embodiments, the API, permeation agent(s), and additional excipients can be incorporated into the pre-mix to form the (aqueous) pharmaceutical formulation of the API. Where required, a pH modifier can be added. In some embodiments, the pharmaceutical formulation can then be scaled up to a desired batch size with solvent, at which point it can be ready for dosing into blister trays containing preformed molds or pockets.

In some embodiments, the pharmaceutical formulation can be prepared by a dry mix. In some embodiments, the API may be dry-mixed with the permeation agent(s), and the dry mixture may be incorporated into the base matrix with the remaining excipients to form the pharmaceutical formulation. In some embodiments, the API may be dry-mixed with the permeation agent(s), and the dry blend may then be mixed with a portion of the base matrix to form an intermediate API suspension that may be incorporated into the remaining base matrix with the remaining excipients to form the pharmaceutical formulation. In some embodiments, the API may be dry-mixed with the permeation agent(s), and the dry blend may be mixed with an appropriate amount of solvent. A portion of base matrix may be incorporated into the API mixture. Multiple base matrix portions may then be added until all of the base matrix has been added.

As explained in detail in the Examples herein, Applicant has discovered suitable permeation enhancers for aiding pre-gastric absorption for a given BCS Class active pharmaceutical ingredient in a pharmaceutical composition/formulation. In some embodiments, the API can be a Class II or Class III API. In some embodiments, the BCS Class II API can be a practically insoluble Class II API. In some embodiments, the practically insoluble BCS Class II API can have a solubility of less than 0.1 mg/mL and/or a lipophilicity with a log P value of greater than 2.5.

APIs belonging to BCS Classes II and III were selected for analysis. The BCS system was developed to provide a scientific approach for classifying APIs based on their aqueous solubility in relation to dose and intestinal permeability in combination with the dissolution properties of an oral immediate release dosage form. Solubility and permeability are API-specific properties, while dissolution is product-specific. According to BCS, APIs are categorized into high and low solubility and permeability classes. Class I APIs have high permeability and high solubility, Class II APIs have low solubility and high permeability, Class III APIs have high solubility and low permeability, and Class IV APIs have low solubility and low permeability. APIs are considered highly soluble when the highest dose strength is soluble in 250 ml or less of aqueous medium at 37° C. with a pH between 1-7.5 as per USFDA guidelines, 1.2-6.8 as per WHO guidelines, and 1-8 as per EMEA guidelines. The BCS definition of aqueous solubility differs from that of “intrinsic solubility,” which reflects the equilibrium aqueous solubility of an API. For acids and bases, intrinsic solubility represents the concentration of the unionized species in a saturated solution at the pH value where that compound is fully unionized.

Examples of practically insoluble BCS Class II APIs include, but are not limited to, carvedilol, aripipazole, asenapine atorvastatin, benidipine HCl, bicalutamide, buspirone, cefditoren pivoxil, cilostazol, citalopram, clotrimazole, clozapine, danazol, digoxin, domperidone, ebastine, etomidate, felodipine, flufenamic acid, flunarizine, flurbiprofen, glibenclamide, glimepiride, glyburide, haloperidol, hydroxyzine, isradipine, ketoprofen, loratadine, lorazepam, lovastatin, nicergoline, nifedipine, nivalipine, nimodipine, nitrendipine, olanzepine, oxatomide, pentazocine, pioglitazone, raloxifene, selegiline, simvastatin, sirolimus, spironolactone, tacrolimus, tamoxifen, testosterone, torsemide, trepostinil, verapamil, or combinations thereof.

In some embodiments, the BCS Class II API can be a very slightly soluble BCS Class II API. In some embodiments, the very slightly soluble BCS Class II API can have a solubility of 0.1-1 mg/mL and/or a lipophilicity with a log P value of 1-2.5. Examples of very slightly soluble BCS Class II APIs include, but are not limited to, piroxicam, allopurinol, apomorphine, cyclosporine, dapsone, diazoxide, diacoumarol, diclofenac, meloxicam, ondansetron, phenazopyridine, prednisone, triamterene, trimethoprim, or combinations thereof.

In some embodiments, the BCS Class III API can be a slightly soluble BCS Class III API. In some embodiments, the slightly soluble BCS Class III API can have a solubility of 1-10 mg/mL and/or a lipophilicity with a log P value less than 1. Examples of slightly soluble BCS Class III API include, but are not limited to, famotidine, amlodipine, blacofen, ceftazidime, dacarbazine, gimeracil, oteracil potassium, tetracycline, or combinations thereof.

In some embodiments, the BCS Class III API can be a sparingly soluble BCS Class III API. In some embodiments, the sparingly soluble BCS Class III API can have a solubility of 10-33 mg/mL and/or a lipophilicity with a log P value less than 1. Examples of sparingly soluble BCS Class III API include, but are not limited to, atenolol, acarbose, amiloride, azacytidine, benznidazole, carbidopa, cefazolin, cefoxitin, cefuroxime, desmopressin acetate hydrate, enalapril acetate, hydralazine, ketorolac, imidapril HCl, latamoxef, lisinopril, midodrine HCl, miglitol, minocyline HCl, morphine, penciclovir, pentostatin, pirenzepine, pramipexole, risedronate sodium hydrate, stavudine, terbutaline, zalcitabine, or combinations thereof.

In some embodiments, the API is included in the pharmaceutical formulations and compositions (e.g., dosage forms) disclosed herein in an amount, which is sufficient to render it pharmaceutically effective when provided in a pharmaceutical composition. A person of skill in the art can readily determine the pharmaceutically effective amount for a given disease or infection based on, among other facts, age and weight of the patient to whom the pharmaceutical composition will be administered. In some embodiments, the pharmaceutical formulation can include at least about 0.05 wt. %, at least about 0.1 wt. %, at least about 0.25 wt. %, at least about 0.5 wt. %, at least about 0.75 wt. %, at least about 1 wt. %, at least about 2 wt. %, at least about 3 wt. %, at least about 4 wt. %, or at least about 5 wt. % API. In some embodiments, the pharmaceutical formulation can include at most about 10 wt. %, at most about 7 wt. %, at most about 5 wt. %, at most about 4.5 wt. %, at most about 4 wt. %, at most about 3.5 wt. %, at most about 3 wt. %, at most about 2.5 wt. %, at most about 2 wt. %, at most about 1.5 wt. %, at most about 1 wt. %, at most about 0.75 wt. %, or at most about 0.5 wt. % API. In some embodiments, the pharmaceutical formulation can include about 0.01-10 wt. % or about 0.1-5 wt. % API.

As shown herein, Applicant discovered that for a practically insoluble (e.g., solubility less than 0.1 mg/mL and Log P value greater than 2.5) BCS Class II APIs, the following permeation enhancers can effectively enhance permeation for these given APIs: counterions, pH modifiers, surfactants having a hydrophilic lipophilic balance (HLB) greater than 10, bile salts, a micelles, a fatty acids, or combinations thereof. As such, at least one of these permeation enhancers can be added with the practically insoluble BCS Class II API (and the base matrix) to form the pharmaceutical formulation.

In addition, Applicant discovered that for very slightly soluble (solubility of 0.1-1 mg/mL and a log P value of 1-2.5) BCS Class II APIs, the following permeation enhancers can effectively enhance permeation for these given APIs: counterions, pH modifiers, surfactants having a hydrophilic lipophilic balance (HLB) greater than 10, bile salts, micelles, or combinations thereof. As such, at least one of these permeation enhancers can be added with the very slightly BCS Class II API (and the base matrix) to form the pharmaceutical formulation. Furthermore, Applicant also discovered that fatty acids and surfactants having a hydrophilic lipophilic balance (HLB) less than 10 can inhibit permeation of the very slightly soluble BCS Class II APIs.

Applicant also discovered that for slightly soluble (solubility of 1-10 mg/mL and Log P value less than 1) BCS Class III APIs, the following permeation enhancers can effectively enhance permeation for these given APIs: counterions, bile salts, pH modifiers, or combinations thereof. As such, at least one of these permeation enhancers can be added with the slightly soluble BCS Class III API (and the base matrix) to form the pharmaceutical formulation. Further, Applicant discovered that micelles and surfactants having an HLB greater than 10 can have no enhancement or only slightly enhance permeation of the slightly soluble BCS Class III APIs.

Lastly, Applicant discovered that for sparingly soluble (solubility of 10-33 mg/mL and Log P less than 1) BCS Class III APIs, the following permeation enhancers can effectively enhance permeation for these given APIs: counterions, pH modifiers, or combinations thereof. As such, at least one of these permeation enhancers can be added with the sparingly soluble BCS Class III API (and the base matrix) to form the pharmaceutical formulation. Furthermore, Applicant discovered that surfactants (both HLB>10 and HLB<10), bile salts, and micelles can have no enhancement of the permeation of sparingly soluble BCS Class III APIs.

As explained above, the permeation enhancer for the practically or very slightly soluble BCS Class II API can include a surfactant. In some embodiments, the surfactant can be at least one of a cationic surfactant (e.g., cetyl trimethylammonium bromide (CTAB), decyltrimethyl ammonium bromide, benzyldimethyl dodecyl ammonium chloride, myristyltrimethyl ammonium chloride, benzalkonium chloride, benzethonium chloride, and/or cetylpyridinium chloride), an anionic surfactant (e.g., sodium lauryl sulfate, sodium dodecl sulphate, sodium octyl sulphate), a hydrophilic surfactant (e.g., polyoxyethylene-20 sorbitan monolaurate (polysorbate 20/Tween20), polyoxyethylene-4 sorbitan monolaurate (Tween 21), polyoxyethylene-20 sorbitan monostearate (polysorbate 60/Tween 60), polyoxyethylene-4 sorbitan monostearate (Tween 61), polyoxyethylene-20 sorbitan monoleate (polysorbate 80/Tween 80), polyoxyethylene-5 sorbitan monoleate (Tween 81)), a lipophilic surfactant (e.g., sorbitan monoleate (Span 80), sorbitan monostearate (span 60), sorbitan monoplamitate (Span 40), and/or sorbitan monolaurate (Span 20)), a polymeric surfactant (e.g., polyoxyethylene (POE)—polyoxypropylene (POP) block copolymer (Poloxamer 124, 188, 407→Pluronic L44, F68, F127)), a glycosidic surfactant (e.g., Dodecyl-B-D-Maltoside, tetradecylmaltoside, tridecylmaltoside, decyl β-D-maltopyranosid/dodecylmaltoside (DDM)), or combinations thereof. In some embodiments, the surfactant can be a surfactant with a hydrophilic lipophilic balance (HLB) greater than 10. The HLB values can be provided from the material data sheets of the various surfactants.

In some embodiments, the pharmaceutical formulation can include at least about 0.01 wt. %, at least about 0.02 wt. %, at least about 0.025 wt. %, at least about 0.05 wt. %, at least about 0.075 wt. %, at least about 0.1 wt. %, at least about 0.25 wt. %, at least about 0.3 wt. %, at least about 0.4 wt. %, at least about 0.5 wt. %, at least about 0.75 wt. %, at least about 1 wt. %, at least about 1.5 wt. %, at least about 2 wt. %, at least about 2.5 wt. %, at least about 3 wt. %, at least about 3.5 wt. %, or at least about 4 wt. % surfactants. In some embodiments, the pharmaceutical formulation can include at most about 5 wt. %, at most about 4.5 wt. %, at most about 4 wt. %, at most about 3.5 wt. %, at most about 3 wt. %, at most about 2.5 wt. %, at most about 2 wt. %, at most about 1.5 wt. %, at most about 1 wt. %, at most about 0.75 wt. %, at most about 0.5 wt. %, at most about 0.4 wt. %, at most about 0.3 wt. %, at most about 0.25 wt. %, at most about 0.1 wt. %, at most about 0.075 wt. %, at most about 0.05 wt. %, or at most about 0.025 wt. %. surfactants. In some embodiments, the pharmaceutical formulation can include about 0.01-5 wt. %, about 0.01-4 wt. %, or about 0.025-3.5 wt. % surfactants. In some embodiments, the pharmaceutical formulation has a critical micellar concentration (CMC) (in mg/mL) of surfactants of at least about 0.1×CMC (meaning 0.1 times its CMC), at least about 0.2×CMC, at least about 0.5×CMC, at least about 1×CMC, at least about 1.5×CMC, at least about 2×CMC, at least about 5×CMC, at least about 10×CMC, at least about 15×CMC, at least about 20×CMC, or at least about 25×CMC. In some embodiments, the pharmaceutical formulation has a CMC of surfactants of at most about 30×CMC, at most about 25×CMC at most about 20×CMC, at most about 15×CMC, at most about 10×CMC, at most about 5×CMC, at most about 3×CMC, at most about 2×CMC, at most about 1.5×CMC, at most about 1×CMC, at most about 0.5×CMC, or at most about 0.2×CMC. In some embodiments, the pharmaceutical formulation has a CMC of surfactants of about 0.1-30×CMC, about 0.5-2×CMC, or about 0.5-1×CMC. In some embodiments, the CMC can be determined according to the methods listed in the article Surfactant Self-Assembling and Critical Micelle Concentration: One Approach Fits All?, by Diego Romano Perinelli et al., Langmuir 2020, 36, 5745-4753, which is hereby incorporated by reference in its entirety.

In some embodiments, the pharmaceutical formulation has a molar ratio of API to surfactant of at least about 1:10, at least about 1:6, at least about 1:5, at least about 1:3, at least about 1:2, or at least about 1:1. In some embodiments, the pharmaceutical formulation has molar ratio of API to surfactant of at most about 10:1, at most about 6:1, at most about 5:1, at most about 3:1, at most about 2:1, or at most about 1:1. In some embodiments, the pharmaceutical formulation has a molar ratio of API to surfactant of about 10:1 to 1:10, about 6:1 to 1:6, about 5:1 to 1:5, about 2:1 to 1:2, or about 1:1.

As explained above, the permeation enhancer for the practically insoluble or very slightly soluble BCS Class II API or permeation enhancer for the slightly soluble BCS Class III API can include bile salts. Examples of bile salts can include, but are not limited to, sodium cholate hydrate, sodium glycodeoxycholate, sodium deoxycholate, sodium taurocholate, sodium taurodeoxycholate, sodium glycocholate, sodium glyodeoxycholate, sodium cholate, sodium ursodeoxycholate, sodium chenodeoxycholate, sodium taurochenodeoxycholate, sodium glycol cheno deoxycholate, sodium cholysaroosinate, sodium N-methyl taurocholate, sodium litocholate, or combinations thereof. In some embodiments, the pharmaceutical formulation can include at least about 0.01 wt. %, at least about 0.02 wt. %, at least about 0.025 wt. %, at least about 0.05 wt. %, at least about 0.075 wt. %, at least about 0.1 wt. %, at least about 0.25 wt. %, at least about 0.3 wt. %, at least about 0.4 wt. %, at least about 0.5 wt. %, at least about 0.75 wt. %, at least about 1 wt. %, at least about 1.5 wt. %, at least about 2 wt. %, at least about 2.5 wt. %, at least about 3 wt. %, at least about 3.5 wt. %, or at least about 4 wt. % bile salts. In some embodiments, the pharmaceutical formulation can include at most about 5 wt. %, at most about 4.5 wt. %, at most about 4 wt. %, at most about 3.5 wt. %, at most about 3 wt. %, at most about 2.5 wt. %, at most about 2 wt. %, at most about 1.5 wt. %, at most about 1 wt. %, at most about 0.75 wt. %, at most about 0.5 wt. %, at most about 0.4 wt. %, at most about 0.3 wt. %, at most about 0.25 wt. %, at most about 0.1 wt. %, at most about 0.075 wt. %, at most about 0.05 wt. %, or at most about 0.025 wt. %. bile salts. In some embodiments, the pharmaceutical formulation can include about 0.01-5 wt. %, about 0.1-4 wt. %, or about 0.3-3 wt. % bile salts. In some embodiments, the pharmaceutical formulation has a critical micellar concentration (CMC) (in mg/mL) of bile salts of at least about 0.1×CMC, at least about 0.2×CMC, at least about 0.5×CMC, at least about 1×CMC, at least about 1.5×CMC, at least about 2×CMC, at least about 5×CMC, or at least about 10×CMC. In some embodiments, the pharmaceutical formulation has a CMC of bile salts of at most about at most about 15×CMC, at most about 10×CMC, at most about 5×CMC, at most about 3×CMC, at most about 2×CMC, at most about 1.5×CMC, at most about 1×CMC, or at most about 0.5×CMC. In some embodiments, the pharmaceutical formulation has a CMC of bile salts of about 0.1-10×CMC, about 0.2-8×CMC, or about 0.5-4×CMC. In some embodiments, the pharmaceutical formulation has a molar ratio of API to bile salts of at least about 1:20, at least about 1:15, at least about 1:10, at least about 1:6, at least about 1:5, at least about 1:3, at least about 1:2, or at least about 1:1. In some embodiments, the pharmaceutical formulation has molar ratio of API to bile salts of at most about 20:1, at most about 15:1, at most about 10:1, at most about 6:1, at most about 5:1, at most about 3:1, at most about 2:1, or at most about 1:1. In some embodiments, the pharmaceutical formulation has a molar ratio of API to bile salts of about 15:1 to 1:15, about 10:1 to 1:10, about 5:1 to 1:5, about 2:1 to 1:2, or about 1:1.

As explained above, the permeation enhancer for the practically insoluble BCS Class II API can include fatty acids. In some embodiments, the fatty acid can be saturated and/or unsaturated. Examples of fatty acids can include, but are not limited to, capric acid, sucrose fatty acid esters (e.g., sucrose monopalmitate), caproic acid, caprylic acid, lauric acid, myristic acid, palmitic acid, stearic acid, arachidic acid, behenic acid, palmitoleic acid, oleic acid, linoleic acid, arachidonic acid, or combinations thereof. In some embodiments, the pharmaceutical formulation can include at least about 0.01 wt. %, at least about 0.02 wt. %, at least about 0.025 wt. %, at least about 0.03 wt. %, at least about 0.05 wt. %, at least about 0.075 wt. %, at least about 0.1 wt. %, at least about 0.25 wt. %, at least about 0.3 wt. %, at least about 0.4 wt. %, at least about 0.5 wt. %, at least about 0.75 wt. %, at least about 1 wt. %, at least about 1.5 wt. %, at least about 2 wt. %, at least about 2.5 wt. %, at least about 3 wt. %, at least about 3.5 wt. %, or at least about 4 wt. % fatty acids. In some embodiments, the pharmaceutical formulation can include at most about 5 wt. %, at most about 4.5 wt. %, at most about 4 wt. %, at most about 3.5 wt. %, at most about 3 wt. %, at most about 2.5 wt. %, at most about 2 wt. %, at most about 1.5 wt. %, at most about 1 wt. %, at most about 0.75 wt. %, at most about 0.5 wt. %, at most about 0.4 wt. %, at most about 0.3 wt. %, at most about 0.25 wt. %, at most about 0.15 wt. %, at most about 0.1 wt. %, at most about 0.075 wt. %, at most about 0.05 wt. %, or at most about 0.025 wt. %. fatty acids. In some embodiments, the pharmaceutical formulation can include about 0.01-5 wt. %, about 0.1-1 wt. %, or about 0.03-0.15 wt. % fatty acids. In some embodiments, the pharmaceutical formulation has a molar ratio of API to fatty acids of at least about 1:5, at least about 1:3, at least about 1:2, or at least about 1:1. In some embodiments, the pharmaceutical formulation has molar ratio of API to fatty acids of at most about 5:1, at most about 3:1, at most about 2:1, or at most about 1:1. In some embodiments, the pharmaceutical formulation has a molar ratio of API to fatty acids of about 5:1 to 1:5, about 2:1 to 1:2, or about 1:1.

As explained above, the permeation enhancer for the practically insoluble or very slightly soluble BCS Class II API or slightly soluble or sparingly soluble BCS Class III API can include counterions. In some embodiments, the ionized counterion can hold an opposite charge to that of the ionized drug molecule at about 1 pH unit, about 1.5 pH units, or 2 pH units from the pKa of the counterions and drug molecule for full ionization. In some embodiments, the difference in pKa between the API and counterion (DpKa) can be greater than 5, greater than 6, or greater than 7. In some embodiments, the counterions can be amino acids such as weak basic, strong basic, basic, acidic, and/or neutral amino acids. Examples of counterions can include, but are not limited to, arginine, histidine, lysine, leucine, glutamic acid, aspartic acid, benzoic acid, glycine, alanine, or combinations thereof. In some embodiments, the pharmaceutical formulation can include at least about 0.01 wt. %, at least about 0.02 wt. %, at least about 0.025 wt. %, at least about 0.03 wt. %, at least about 0.04 wt. %, at least about 0.05 wt. %, at least about 0.075 wt. %, at least about 0.1 wt. %, at least about 0.25 wt. %, at least about 0.3 wt. %, at least about 0.4 wt. %, at least about 0.5 wt. %, at least about 0.75 wt. %, at least about 1 wt. %, at least about 1.5 wt. %, at least about 2 wt. %, at least about 2.5 wt. %, at least about 3 wt. %, at least about 3.5 wt. %, or at least about 4 wt. % counterions. In some embodiments, the pharmaceutical formulation can include at most about 5 wt. %, at most about 4.5 wt. %, at most about 4 wt. %, at most about 3.5 wt. %, at most about 3 wt. %, at most about 2.5 wt. %, at most about 2 wt. %, at most about 1.5 wt. %, at most about 1 wt. %, at most about 0.75 wt. %, at most about 0.5 wt. %, at most about 0.4 wt. %, at most about 0.3 wt. %, at most about 0.25 wt. %, at most about 0.15 wt. %, at most about 0.1 wt. %, at most about 0.075 wt. %, at most about 0.05 wt. %, or at most about 0.025 wt. %. counterions. In some embodiments, the pharmaceutical formulation can include about 0.01-5 wt. %, about 0.1-5 wt. %, or about 0.04-4 wt. % counterions. In some embodiments, the pharmaceutical formulation has a molar ratio of API to counterions of at least about 1:10, at least about 1:6, at least about 1:5, at least about 1:3, at least about 1:2, or at least about 1:1. In some embodiments, the pharmaceutical formulation has molar ratio of API to counterions of at most about 10:1, at most about 6:1, at most about 5:1, at most about 3:1, at most about 2:1, or at most about 1:1. In some embodiments, the pharmaceutical formulation has a molar ratio of API to counterions of about 6:1 to 1:6, about 3:1 to 1:3, about 2:1 to 1:2, or about 1:1.

In some embodiments, the permeation enhancer can include a pH modifier. In some embodiments, the enhancement or inhibition of enhancement of the pH modifier can depend on the effect of the un-ionization fraction of the API. In some embodiments, the pH modifier can be acidic and/or an alkalizing agent. Examples of pH modifiers can include, but are not limited to, citric acid, malic acid, maliec acid, tartaric acid, lactic acid, fumaric acid, succinic acid, adipic acid, ascorbic acid, sodium hydroxide, magnesium hydroxide, potassium hydroxide, sodium carbonate, magnesium carbonate, potassium carbonate, calcium carbonate, sodium phosphate monobasic, sodium phosphate dibasic, sodium phosphate tribasic, sodium citrate, sodium acetate, or combinations thereof. In some embodiments, the pharmaceutical formulation can include at least about 0.005 wt. %, at least about 0.01 wt. %, at least about 0.02 wt. %, at least about 0.025 wt. %, at least about 0.03 wt. %, at least about 0.04 wt. %, at least about 0.05 wt. %, at least about 0.075 wt. %, at least about 0.1 wt. %, at least about 0.25 wt. %, at least about 0.3 wt. %, at least about 0.4 wt. %, at least about 0.5 wt. %, at least about 0.75 wt. %, at least about 1 wt. %, at least about 1.5 wt. %, at least about 2 wt. %, at least about 2.5 wt. %, at least about 3 wt. %, at least about 3.5 wt. %, or at least about 4 wt. % pH modifiers. In some embodiments, the pharmaceutical formulation can include at most about 5 wt. %, at most about 4.5 wt. %, at most about 4 wt. %, at most about 3.5 wt. %, at most about 3 wt. %, at most about 2.5 wt. %, at most about 2 wt. %, at most about 1.5 wt. %, at most about 1 wt. %, at most about 0.75 wt. %, at most about 0.5 wt. %, at most about 0.4 wt. %, at most about 0.3 wt. %, at most about 0.25 wt. %, at most about 0.15 wt. %, at most about 0.1 wt. %, at most about 0.075 wt. %, at most about 0.05 wt. %, at most about 0.025 wt. %. pH modifiers. In some embodiments, the pharmaceutical formulation can include about 0.005-2.5 wt. % pH modifiers. In some embodiments, the pH of the pharmaceutical formulation can be about 3.5-9.5 or about 4-8.

Once the pharmaceutical formulation was prepared, the pharmaceutical formulation can be dosed into preformed molds. As used herein, “dosed” (or similar terminology) refers to the deposition of a pre-determined aliquot of solution or suspensions. As used herein, “preformed molds” refers to any suitable container or compartment into which an aqueous solution or suspension may be deposited and within which subsequently freeze dried. In some embodiments, the preformed mold is a blister pack with one or more blister pockets. In some embodiments, predetermined aliquots in an amount of about 150-1000 mg or about 500 mg wet filling dosing weight of the pharmaceutical formulation can be metered into preformed molds. In some embodiments, the minimum unit size (e.g., wet fill weight, 150 mg) can be selected to minimize the amount of API in solution proportionally to the unit dose, and therefore its surface area and potential for oxidative degradation in the final dosage form.

The dosed pharmaceutical formulation can then be frozen in the preformed molds. The dosed formulations in the preformed molds can be frozen by any means known in the art. For example, the formulations can be passed through a cryogenic chamber (e.g., liquid nitrogen tunnel). In some embodiments, the freezing temperature and duration of freezing can be varied to ensure that the dosed pharmaceutical formulation was frozen.

The frozen units can then be collected and held in a freezer at a sub-zero temperature appropriate for the formulation prior to freeze-drying. Alternatively, the frozen units may be annealed for a suitable period of time to crystallize the structural former. Structural former crystallization can provide the frozen units with the structural strength to prevent collapse during freeze-drying.

Following the frozen hold, the frozen units can be freeze-dried into pharmaceutical compositions such as dosage forms (e.g., tablets). The freeze-drying cycle can be optimized for individual formulations. During the freeze-drying process, water can removed from the frozen units by sublimation, leaving porous freeze-dried units which can rapidly disintegrate when placed in the oral cavity.

The freeze-dried pharmaceutical compositions can then removed from the freeze-drier and inspected for any defects. The inspected pharmaceutical compositions can then be placed in a storage cabinet at a temperature and humidity-controlled environment ready to be sealed in their preformed blister trays.

Blister trays can be sealed by placing a lidding foil on the preformed trays. The preformed trays can then be cut to form individual blisters containing the freeze-dried pharmaceutical compositions (e.g., ODTs).

In some embodiments, the pharmaceutical compositions (i.e., post lyophilization) can include at least about 20 wt. %, at least about 25 wt. %, at least about 30 wt. %, at least about 35 wt. %, at least about 40 wt. %, or at least about 50 wt. % matrix former. In some embodiments, the pharmaceutical composition includes at most about 65 wt. %, at most about 60 wt. %, at most about 55 wt. %, at most about 50 wt. %, at most about 45 wt. %, or at most about 40 wt. % matrix former. In some embodiments, the pharmaceutical compositions (i.e., post lyophilization) can include about 25-60 wt. %, about 30-60 wt. %, or about 33-56.25 wt. % matrix former. In some embodiments, the pharmaceutical compositions can include at least about 15 wt. %, at least about 20 wt. %, at least about 25 wt. %, at least about 30 wt. %, at least about 35 wt. %, or at least about 40 wt. % structure former. In some embodiments, the pharmaceutical composition can include at most about 50 wt. %, at most about 45 wt. %, at most about 40 wt. %, at most about 35 wt. %, or at most about 30 wt. %, structure former. In some embodiments, the pharmaceutical compositions can include about 20-45 wt. % or about 29-42.4 wt. % structure former.

In some embodiments, the API is included in the pharmaceutical compositions (e.g., dosage forms) disclosed herein in an amount, which is sufficient to render it pharmaceutically effective when provided in a pharmaceutical composition. A person of skill in the art can readily determine the pharmaceutically effective amount for a given disease or infection based on, among other facts, age and weight of the patient to whom the pharmaceutical composition will be administered. In some embodiments, the pharmaceutical composition can include at least about 1 wt. %, at least about 2 wt. %, at least about 3 wt. %, at least about 4 wt. %, at least about 5 wt. %, at least about 10 wt %, at least about 15 wt. %, at least about 20 wt. %, at least about 25 wt. %, at least about 30 wt. %, or at least about 35 wt. % API. In some embodiments, the pharmaceutical composition can include at most about 40 wt. %, at most about 35 wt. %, at most about 30 wt. %, at most about 25 wt. %, at most about 21 wt. %, at most about 20 wt. %, at most about 15 wt. %, at most about 10 wt. %, or at most about 5 wt. % API. In some embodiments, the pharmaceutical composition can include about 1-40 wt. % or about 1.41-21 wt. % API.

As explained herein, Applicant has discovered specific permeation enhancers for specific APIs to be used in pharmaceutical compositions. In situations where surfactants are included as the permeation enhancer, the pharmaceutical compositions (i.e., post lyophilization) can include at least about 0.1 wt. %, at least about 0.5 wt. %, at least about 0.75 wt. %, at least about 1 wt. %, at least about 5 wt. %, at least about 10 wt. %, at least about 15 wt. %, at least about 17 wt. %, or at least about 20 wt. % surfactants. In some embodiments, the pharmaceutical compositions can include at most about 30 wt. %, at most about 25 wt. %, at most about 20 wt. %, at most about 17 wt. %, at most about 15 wt. %. at most about 10 wt. %, or at most about 5 wt. % surfactants. In some embodiments, the pharmaceutical compositions can include 0.8-21.2 wt. % or about 0.14-17 wt. % surfactants.

In some embodiments, the pharmaceutical composition has a molar ratio of API to surfactants of at least about 1:10, at least about 1:6, at least about 1:5, at least about 1:3, at least about 1:2, or at least about 1:1. In some embodiments, the pharmaceutical composition has molar ratio of API to surfactants of at most about 10:1, at most about 6:1, at most about 5:1, at most about 3:1, at most about 2:1, or at most about 1:1. In some embodiments, the pharmaceutical composition has a molar ratio of API to surfactants of about 10:1 to 1:10, about 6:1 to 1:6, about 5:1 to 1:5, about 2:1 to 1:2, or about 1:1.

In situations where bile salts are included as the permeation enhancer, the pharmaceutical compositions (i.e., post lyophilization) can include at least about 0.1 wt. %, at least about 0.5 wt. %, at least about 0.75 wt. %, at least about 1 wt. %, at least about 2 wt. %, at least about 5 wt. %, at least about 10 wt. %, at least about 15 wt. %, or at least about 17 wt. % bile salts. In some embodiments, the pharmaceutical compositions can include at most about 20 wt. %, at most about 17 wt. %, at most about 15 wt. %. at most about 10 wt. %, or at most about 5 wt. % bile salts. In some embodiments, the pharmaceutical compositions can include about 0.14-17 wt. % or about 2.5-17.3 wt. % bile salts.

In some embodiments, the pharmaceutical compositions have a molar ratio of API to bile salts of at least about 1:20, at least about 1:15, at least about 1:10, at least about 1:6, at least about 1:5, at least about 1:3, at least about 1:2, or at least about 1:1. In some embodiments, the pharmaceutical compositions have molar ratio of API to bile salts of at most about 20:1, at most about 15:1, at most about 10:1, at most about 6:1, at most about 5:1, at most about 3:1, at most about 2:1, or at most about 1:1. In some embodiments, the pharmaceutical compositions have a molar ratio of API to bile salts of about 15:1 to 1:15, about 10:1 to 1:10, about 5:1 to 1:5, about 2:1 to 1:2, or about 1:1.

In situations where fatty acids are included as the permeation enhancer, the pharmaceutical compositions (i.e., post lyophilization) can include at least about 0.1 wt. %, at least about 0.5 wt. %, at least about 0.75 wt. %, at least about 1 wt. %, at least about 2 wt. %, at least about 5 wt. %, at least about 10 wt. %, at least about 15 wt. %, or at least about 17 wt. % fatty acids. In some embodiments, the pharmaceutical compositions can include at most about 20 wt. %, at most about 17 wt. %, at most about 15 wt. %. at most about 10 wt. %, or at most about 5 wt. % fatty acids. In some embodiments, the pharmaceutical compositions can include about 0.15-17 wt. % fatty acids. In some embodiments, the pharmaceutical composition has a molar ratio of API to fatty acids of at least about 1:6, at least about 1:5, at least about 1:3, at least about 1:2, or at least about 1:1. In some embodiments, the pharmaceutical composition has molar ratio of API to fatty acids of at most about 6:1, at most about 5:1, at most about 3:1, at most about 2:1, or at most about 1:1. In some embodiments, the pharmaceutical composition has a molar ratio of API to fatty acids of about 5:1 to 1:5, about 3:1 to 1:3, about 2:1 to 1:2, or about 1:1.

In situations where counterions are included as the permeation enhancer, the pharmaceutical compositions (i.e., post lyophilization) can include at least about 0.1 wt. %, at least about 0.5 wt. %, at least about 0.75 wt. %, at least about 1 wt. %, at least about 5 wt. %, at least about 8 wt. %, at least about 10 wt. %, at least about 15 wt. %, at least about 17 wt. %, or at least about 20 wt. % counterions. In some embodiments, the pharmaceutical compositions can include at most about 30 wt. %, at most about 25 wt. %, at most about 20 wt. %, at most about 17 wt. %, at most about 15 wt. %. at most about 10 wt. %, or at most about 5 wt. % counterions. In some embodiments, the pharmaceutical compositions can include about 5-25 wt. %, about 8-22 wt. %, or about 0.1-20 wt. % counterions. In some embodiments, the pharmaceutical composition has a molar ratio of API to counterions of at least about 1:10, at least about 1:6, at least about 1:5, at least about 1:3, at least about 1:2, or at least about 1:1. In some embodiments, the pharmaceutical composition has molar ratio of API to counterions of at most about 10:1, at most about 6:1, at most about 5:1, at most about 3:1, at most about 2:1, or at most about 1:1. In some embodiments, the pharmaceutical composition has a molar ratio of API to counterions of about 6:1 to 1:6, about 3:1 to 1:3, about 2:1 to 1:2, or about 1:1.

As stated above, the permeation enhancer can also include a pH modifier, in some embodiments. In some embodiments, the pharmaceutical composition includes about 0.1-5 wt. %, about 0.1-3 wt. %, about 0.1-2 wt. %, about 0.1-1.5 wt. %, about 0.2-1.5 wt. %, about 0.3-1.2 wt. %, about 0.5-1.1 wt. %, or about 0.5-1 wt. % pH modifier. In some embodiments, the pharmaceutical composition can include at least about 0.1 wt. %, at least about 0.2 wt. %, at least about 0.3 wt. %, at least about 0.4 wt. %, at least about 0.5 wt. %, at least about 0.58 wt. %, at least about 0.75 wt. %, or at least about 1 wt. % pH modifier. In some embodiments, the pharmaceutical composition can include at most about 5 wt. %, at most about 3 wt. %, at most about 2 wt. %, at most about 1.5 wt. %, at most about 1.25 wt. %, at most about 1 wt. %, at most about 0.99 wt. %, or at most about 0.75 wt. % pH modifier. In some embodiments, the pharmaceutical composition includes 0.1-5 wt. % pH modifier. The pH modifier in the pharmaceutical composition is just the pH modifier itself even if the pH modifier was in solution in the pharmaceutical formulation. The freeze-drying process can remove the water from the pH modifier solution.

The pharmaceutical compositions (e.g., dosage forms) can be dissolving dosage forms and accordingly have the distinct advantage of a faster disintegrating time.

Permeability was assessed by quantifying the amount of drug permeated into the receiver compartment at the given time points. % permeability was calculated from the proportion of drug permeated (i.e. in the receiver compartment) when compared to the total amount in the donor compartment (i.e. how much drug was placed in the donor at the start of the studies). The resultant value was then multiplied by 100 to yield a percentage. In some embodiments, percent increase in permeability by adding the permeation agent (when compared to no permeation agent) can be at least about 0.1%, at least about 1%, at least about 5%, at least about 10 wt. %, at least about 15%, at least about 20%, at least about 25%, at least about 30%, at least about 35%, at least about 40%, at least about 45%, or at least about 50%. In some embodiments, percent increase in permeability by adding the permeation agent (when compared to no permeation agent) is at most about 100%, at most about 75%, or at most about 50%.

For the purpose of clarity and a concise description, features are described herein as part of the same or separate embodiments; however, it will be appreciated that the scope of the disclosure includes embodiments having combinations of all or some of the features described.

### EXAMPLES

The following examples are provided as model data representing combinations of API BCS information and sub-classification information (solubility and permeability) and suitable permeation enhancers associated respectively with each of the combinations of API BCS information and sub-classification information.

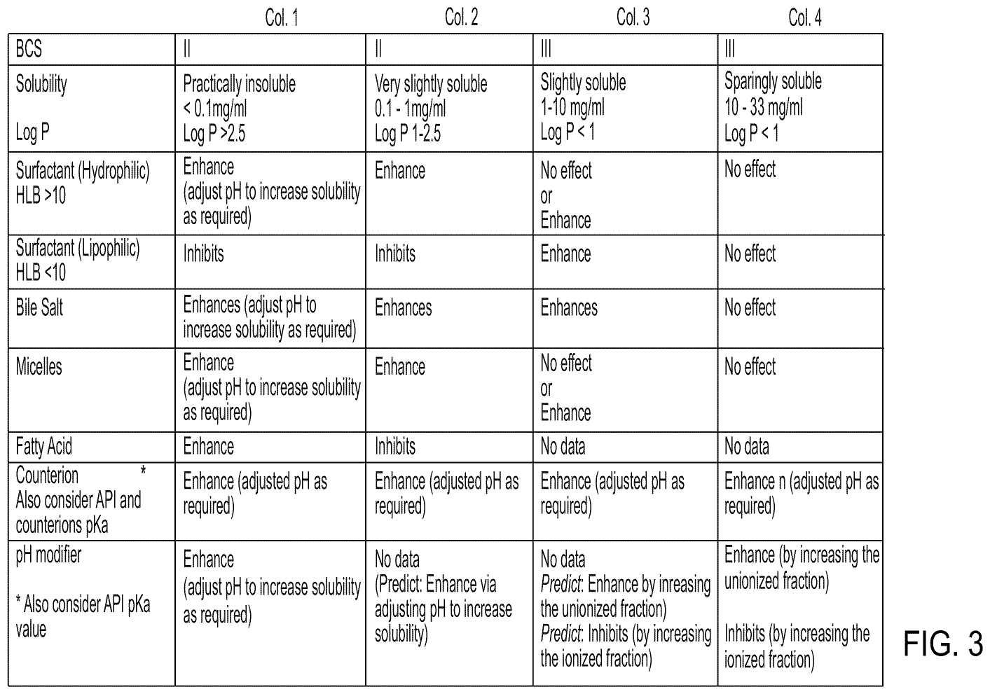

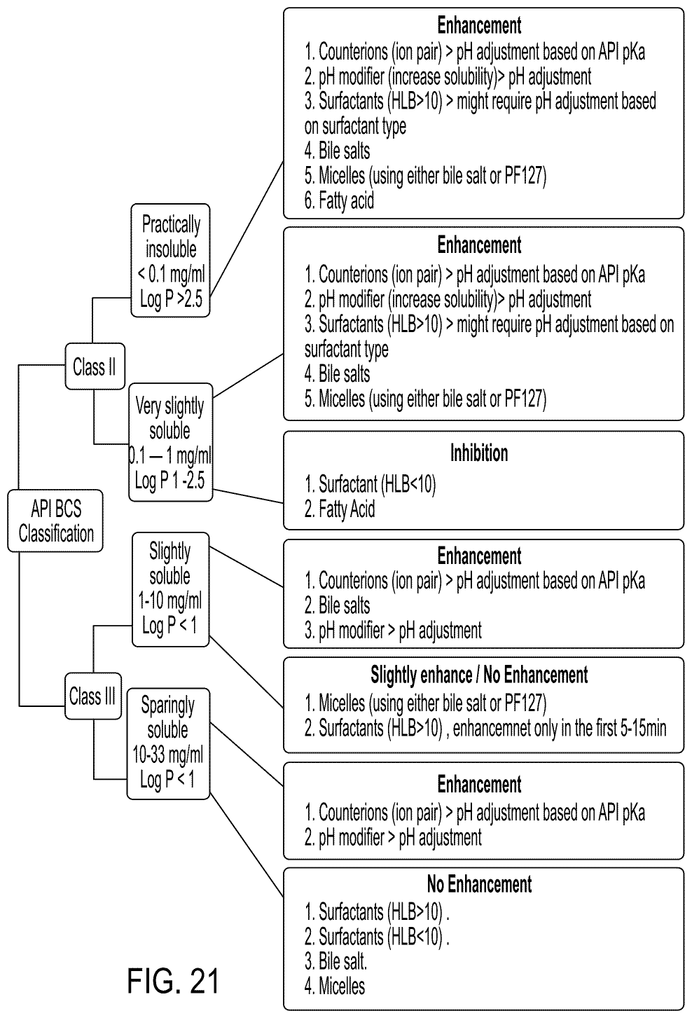

The following procedures are examples of how the data representing combinations of API BCS information and sub-classification information and suitable permeation enhancers associated respectively with each of the combinations of API BCS information and sub-classification information illustrated in FIGS. 3 and 21 were generated.

### Selection of Model APIs

Several model mucosal permeation agents were assessed for their ability to modulate the permeability of four model BCS Class II and Class III APIs for pre-gastric delivery (e.g., via the oral cavity, pharynx, and/or esophagus) by freeze-dried ODTs.

APIs are considered highly permeable when the extent of intestinal systemic absorption of the parent drug plus metabolites in humans is determined to be 90% or more of an administered dose based on a mass-balance determination or in comparison to an intravenous reference dose. The methods for estimating BCS permeability criteria are based on in vivo intestinal absorption or high throughput systems available for examining human intestinal permeability, such as CaCO₂ cells and parallel artificial membrane permeability assay (PAMPA). Thus, they may not be easily extrapolated to pre-gastric permeability. One of the objectives of this experiment was to establish an in vitro permeability testing approach which could be translated to pre-gastric absorption.

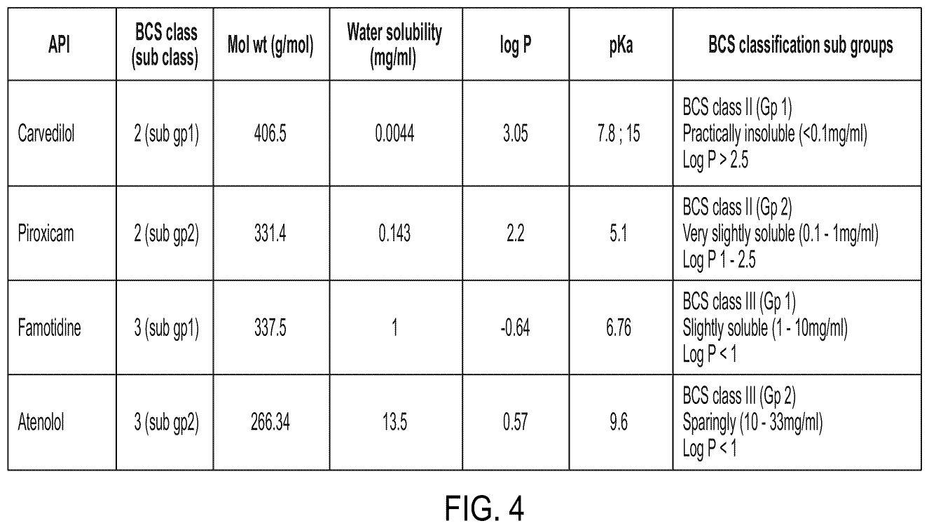

Two model APIs were selected from each of BCS Classes II and III. The BCS Class II APIs used were Piroxicam and Carvedilol. The BCS Class III APIs used were Atenolol and Famotidine. These APIs were selected because they have well-characterized properties (shown in FIG. 4) such as molecular size, solubility, lipophilicity (log P), and ionization properties (pKa). There is also existing pharmacokinetics data on gastrointestinal adsorption of these APIs when delivered through oral dosage forms such as tablets and capsules. However, their pre-gastric absorption is less established.

### Selection of Permeation Agents

Permeation agents including surfactants, bile salts, counterions, fatty acids, and pH modifiers were assessed. The model permeation agents selected are listed in Table 1 below. Permeation agents were selected based on their mode of action, compatibility with the formulation and manufacturing process of a freeze-dried ODT, toxicity to the mucosal membrane, solubilizing capacity, drug release characteristics, and compatibility with numerous APIs and excipients.

Surfactants can be classified according to their polar head group as ionic or non-ionic. Surfactants can exhibit a diverse range of properties such as hydrophilic-lipophilic balance (HLB) values, molecular weights, chain lengths, critical micellar concentrations (CMC), and solubility parameters. Surfactants may enhance solubility and can form micelles when at or above the CMC. Surfactants can also interact with the lipid bilayer of cell membranes, thereby increasing the permeability of an API. Non-ionic surfactants are considered favorable because of their low toxicity. In this experiment, sodium lauryl sulfate was selected as a model ionic surfactant, and Tween 21, Span 80, Pluronics F127 and L44, and Dodecyl-B-D-Maltoside were selected as model non-ionic surfactants.

Bile salts may increase drug permeability due to their amphiphilic properties. When their concentration is above the CMC, they can self-associate in water and form supramolecular aggregates or micelles. To enhance permeability across an epithelial barrier, the bile salts may disrupt the plasma membrane, causing leakage of phospholipids and loosening the intercellular filaments, resulting in decreased integrity of the epithelium and micelle formation with the API. In this experiment, sodium cholate hydrate was selected as a model bile salt.

Fatty acids may increase drug permeability through several processes such as drug solubilization, increasing membrane fluidity, and paracellular transport. In this experiment, capric acid was selected as a model fatty acid.

Counterions may increase drug permeability by forming ion pairs. The ion pairing approach involves complexing a counterion with an oppositely charged drug molecule, resulting in an overall neutral ion pair held together as a single unit by coulombic attractive forces. The choice of counterion is dependent upon the pKa and charge of the ionized drug molecule with which the counterion needs to form the ion pair. In this experiment, arginine, lysine, aspartic acid, benzoic acid, glycine, and alanine were selected as model counterions.

TABLE 1
Model permeation agents assessed

| | | Model Permeation | |
|---|---|---|---|
| Type | | Agent | Proposed Mode of Action |
| Anionic surfactant | | Sodium Lauryl Sulfate | Solubilizes drug. |
| Non- | hydrophilic | POE-(4)-sorbitan | Fluidizes the bilayer membrane. |
| ionic | | monolaurate: | Increases the drug partitioning. |
| surfactant | | Tween 21 | Particulate system (micelles). |
| | lipophilic | Sorbitan monooleate: | |
| | | Span 80 | |
| | polymer | Poloxamer: | Solubilizes drug. |
| | | Pluronic F127 | Fluidizes the bilayer membrane. |
| | | Pluronic L44 | Changes mucus rheology. |
| | | | Particulate systems (micelles). |
| | glycosidic | Dodecyl-B-D-Maltoside | Solubilizes drug. |
| | | (DDM) | Fluidizes the bilayer membrane. |
| | | | Particulate systems (micelles). |
| Bile salt | | Sodium Cholate hydrate | Fluidizes the bilayer membrane. |
| | | | Inhibits mucosal enzymes. |
| | | | Particulate system (micelles). |
| Fatty acid | | Capric acid | Solubilizes drug. |
| | | | Fluidizes membrane. |
| | | | Particulate system (colloidal). |
| pH modifier | | Citric acid | Solubilizes drug. |
| | | | Modifies the unionized vs ionized |
| | | | fractions. |
| Counterion | | Arginine | Ion pairing. |
| | | Lysine | Solubilizes drug. |
| | | Aspartic acid | |
| | | Benzoic acid | |
| | | Glycine | |
| | | Alanine | |

### Cell Culture Setup

Drug permeability of several APIs was assessed using an in-vitro cultured cell method using a TR146 buccal cell line, a well-characterized cell culture model derived from a neck node metastasis of a buccal carcinoma. TR146 cells were grown in a culture medium enriched with nutrients to produce a multi-layer cellular barrier that mimics the buccal epithelium. Cells were seeded on Transwell polymer inserts and allowed to stratify over a period of 25 to 30 days. Media was changed regularly and optimal cell conditions were maintained at 37° C. and 5% CO2 at 98% relative humidity. During cell culture, transepithelial electrical resistance (TEER) was measured every 2 to 3 days. TEER indicates the integrity of the cellular layer in the Transwell inserts. A consistent reading across a study implies that the integrity of the cells remains strong and increases confidence in the results. Once the TEER indicated the cells were fully stratified, the inserts were ready to use in the permeability study.

### Pre-Formulation Assessments of APIs and Permeation Agents

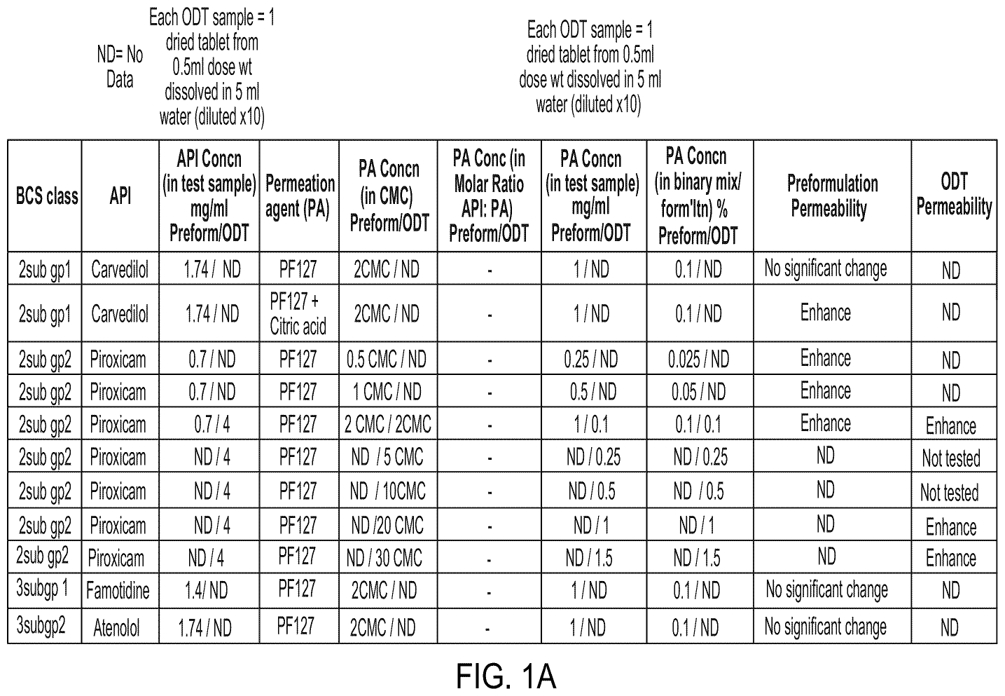

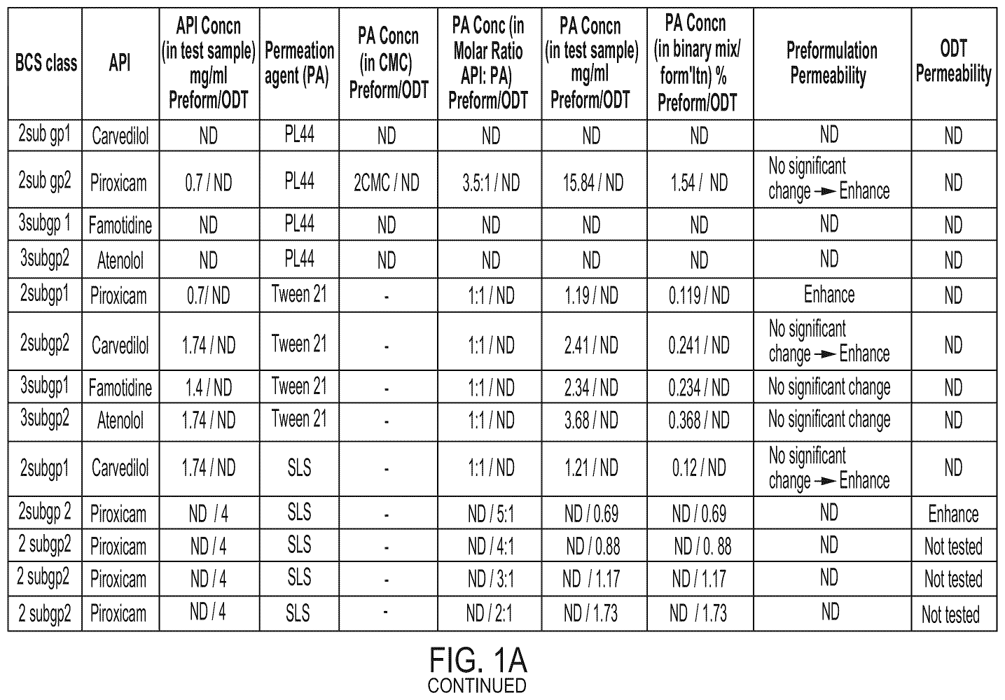

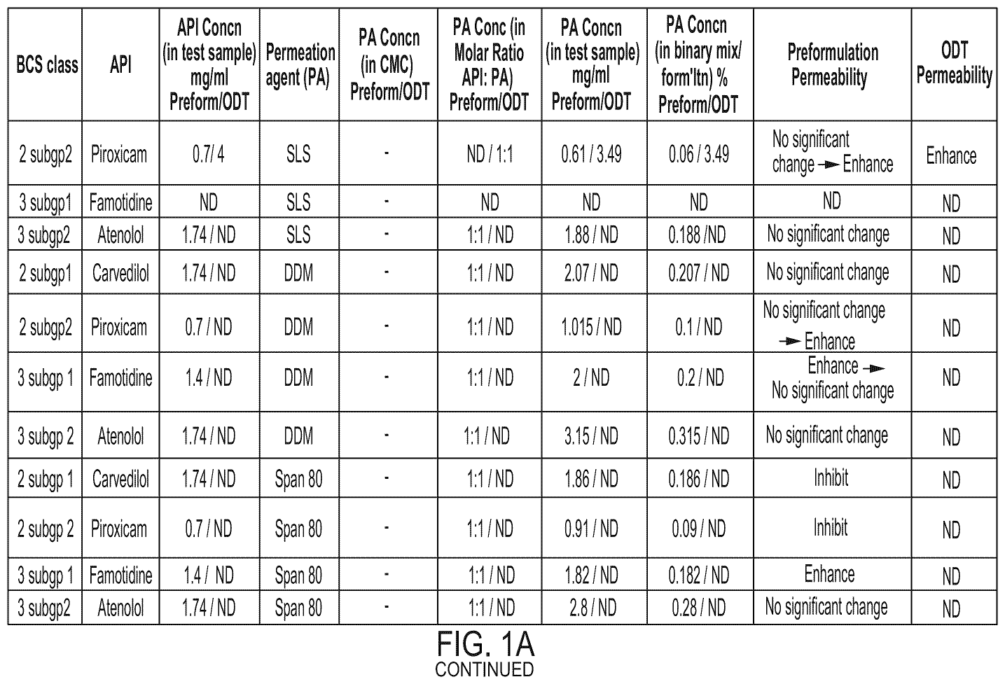

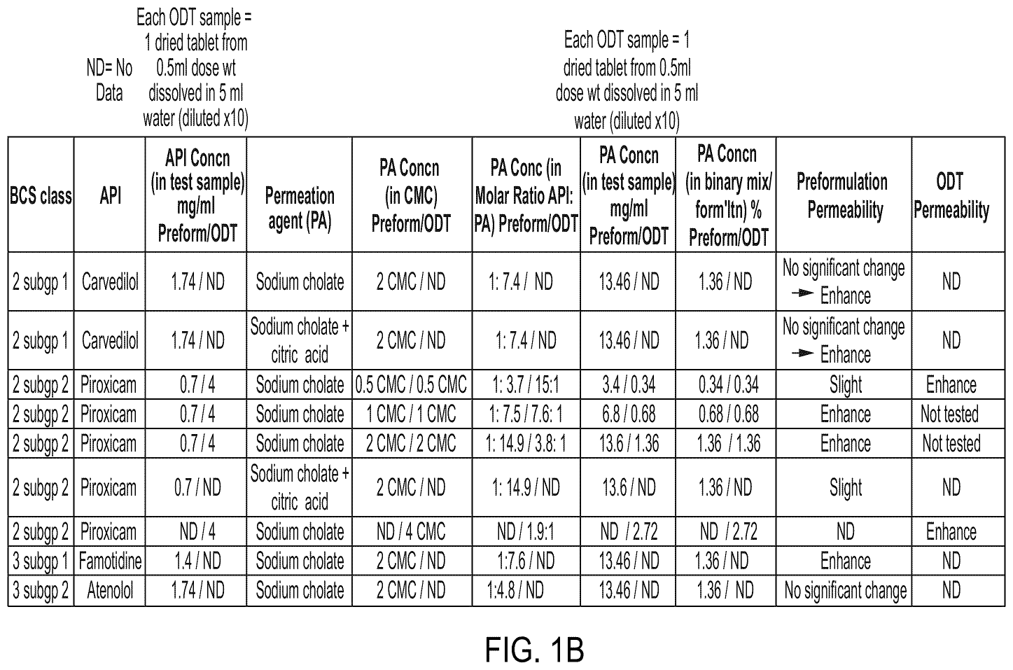

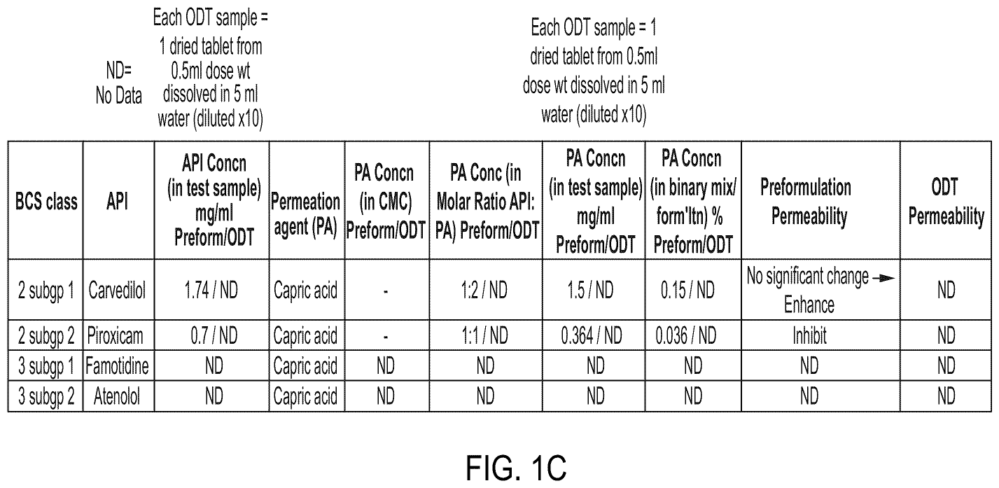

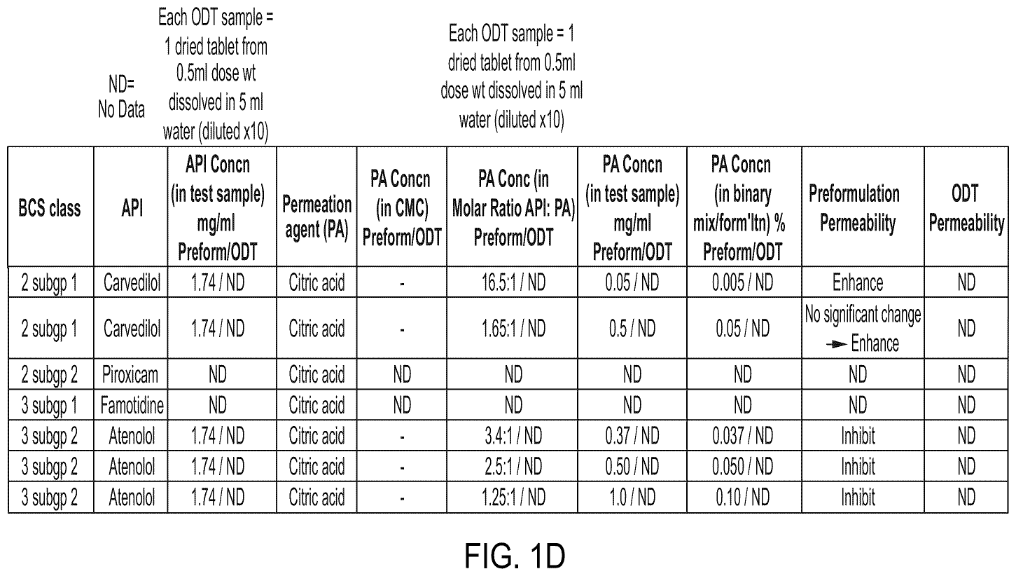

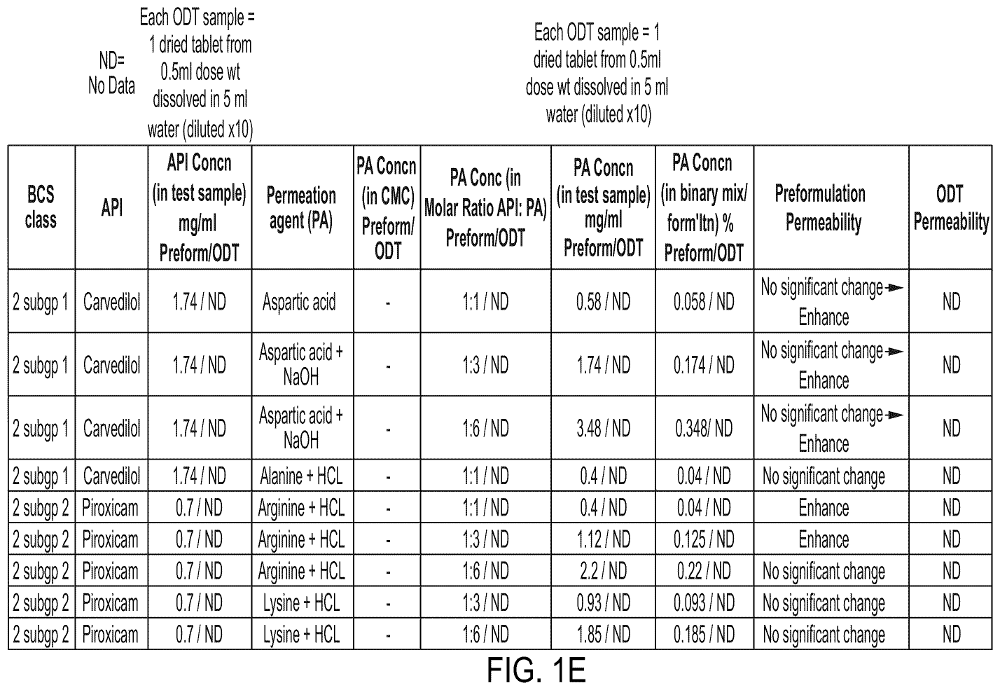

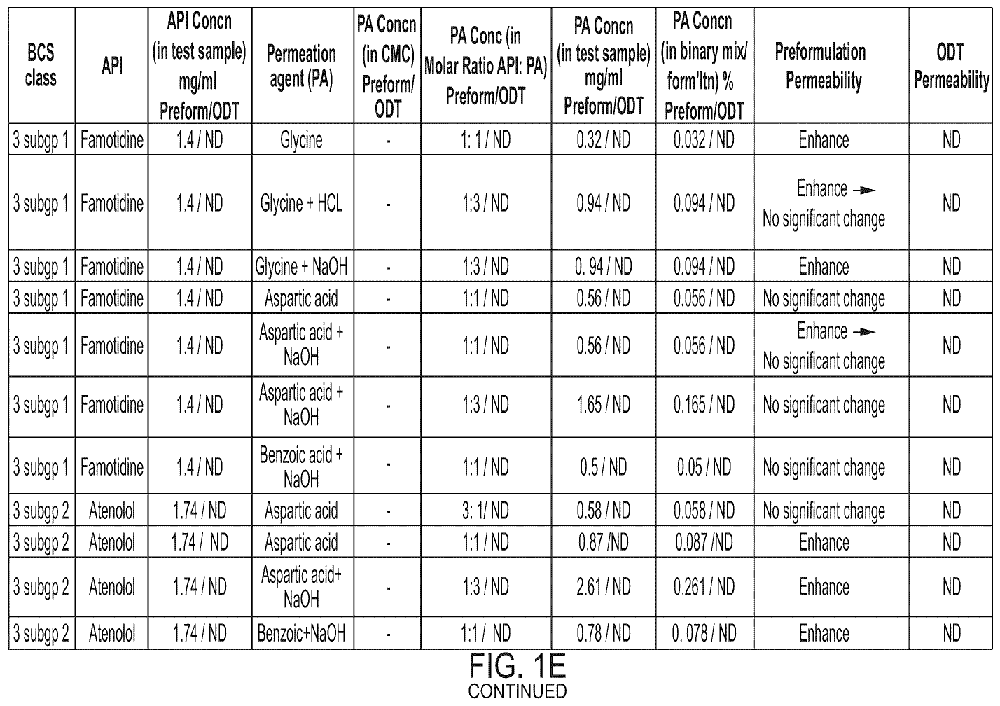

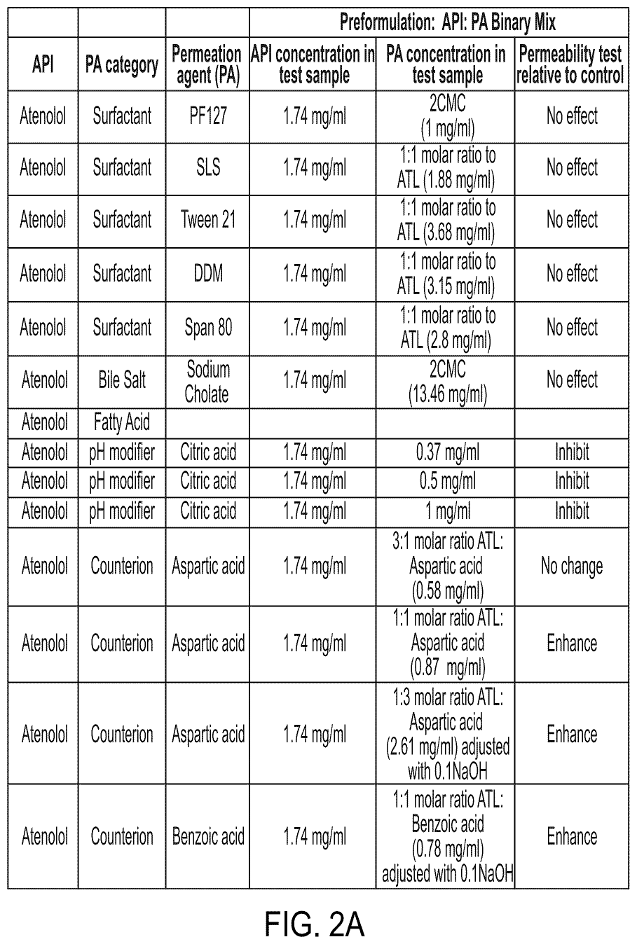

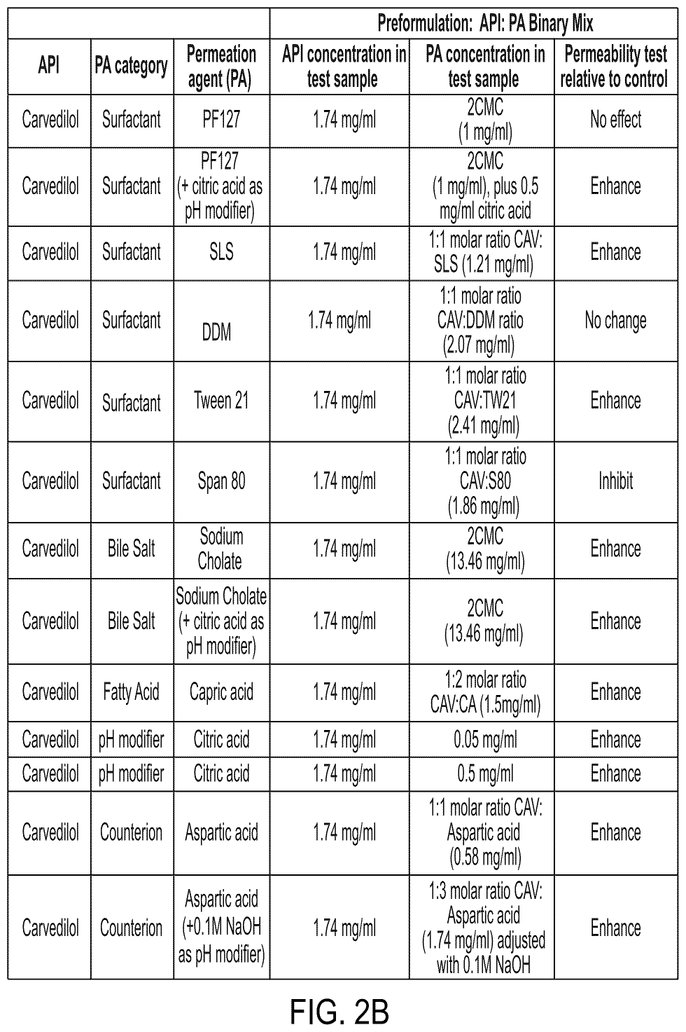

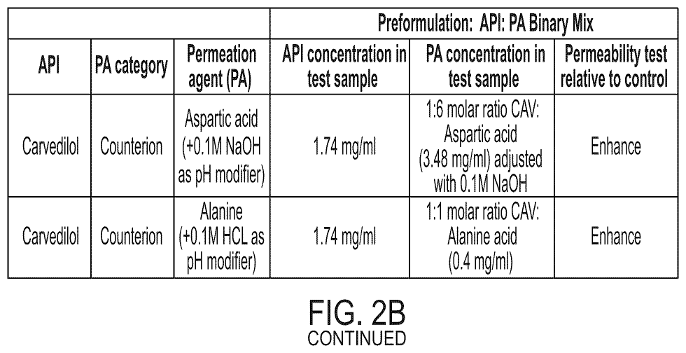

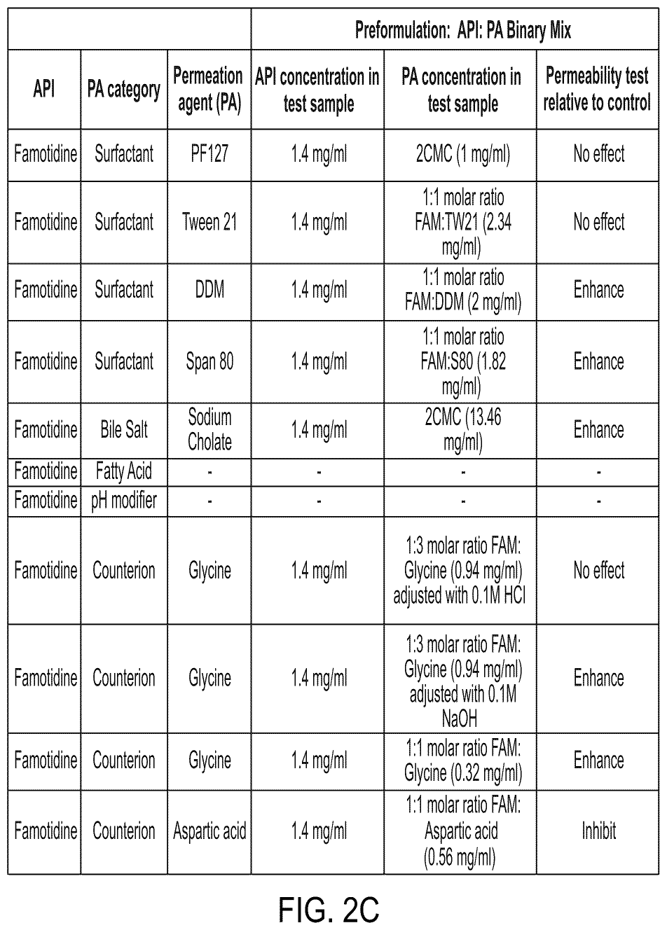

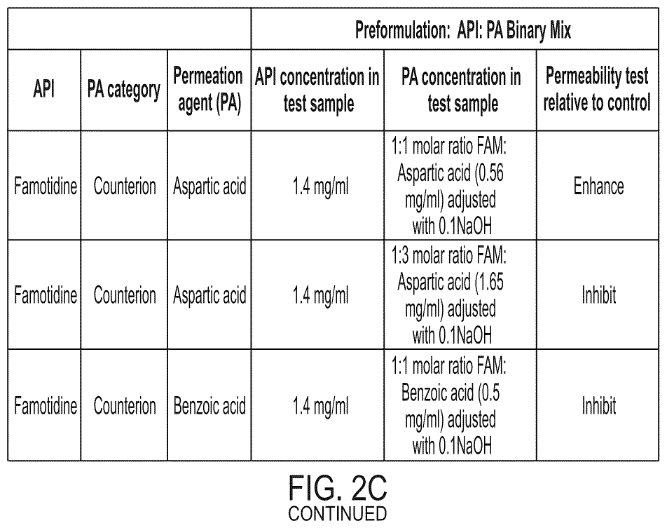

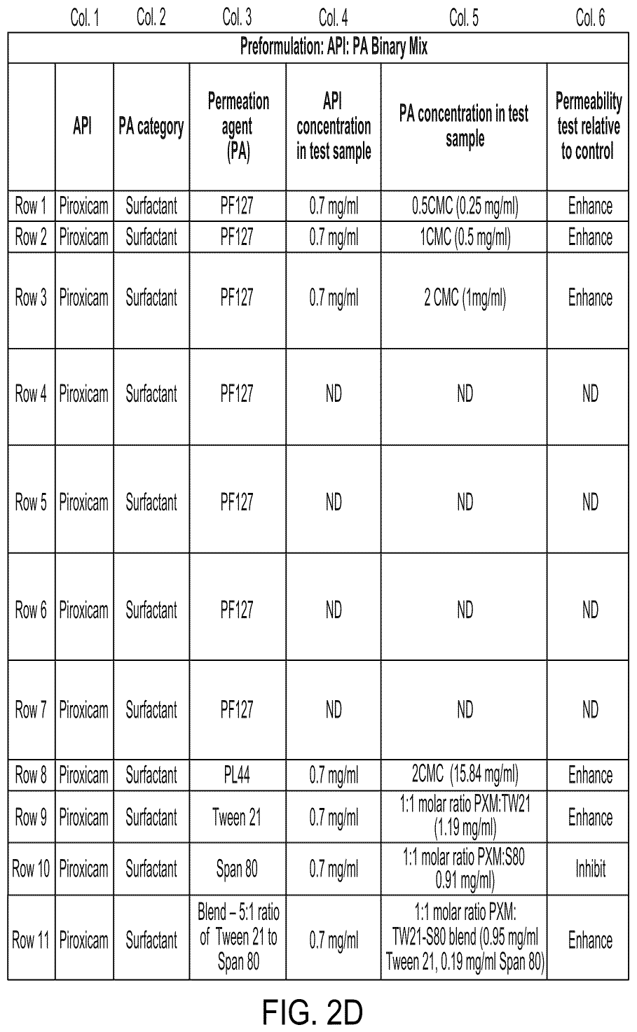

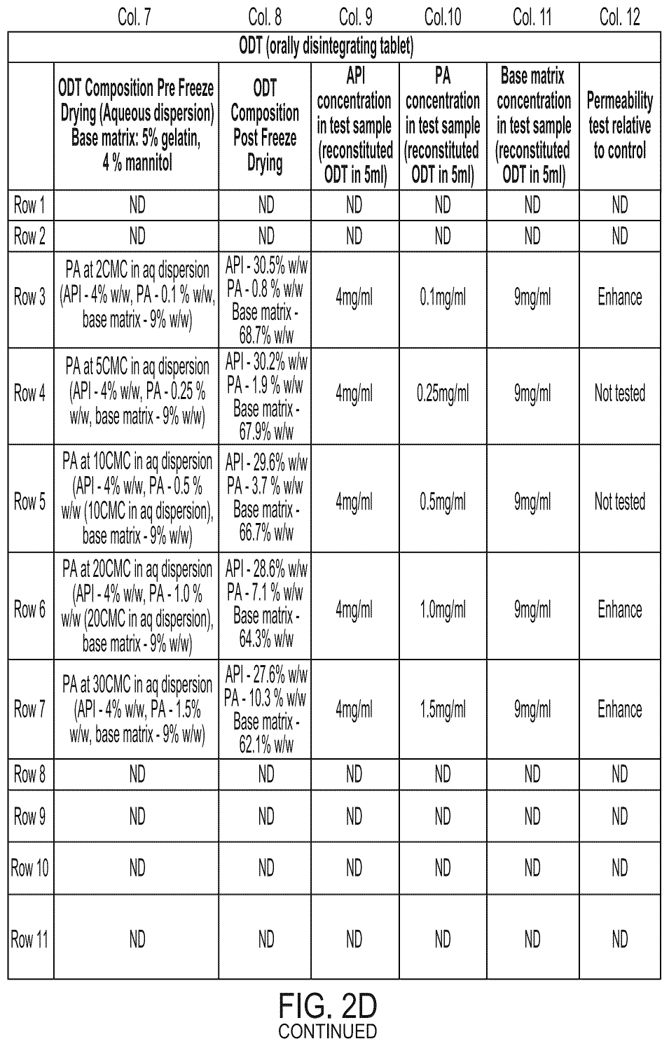

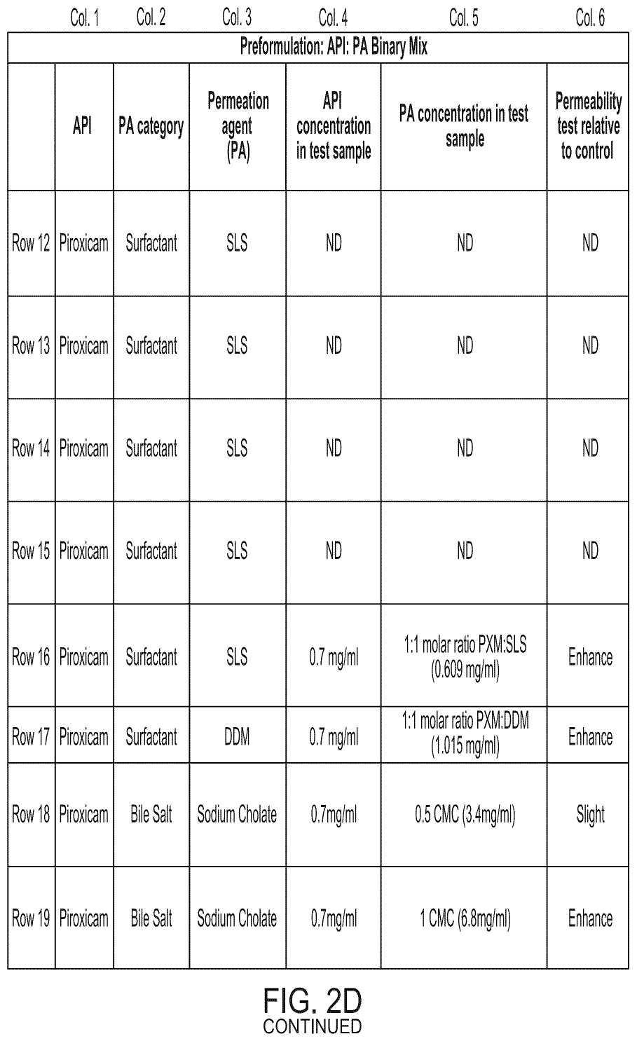

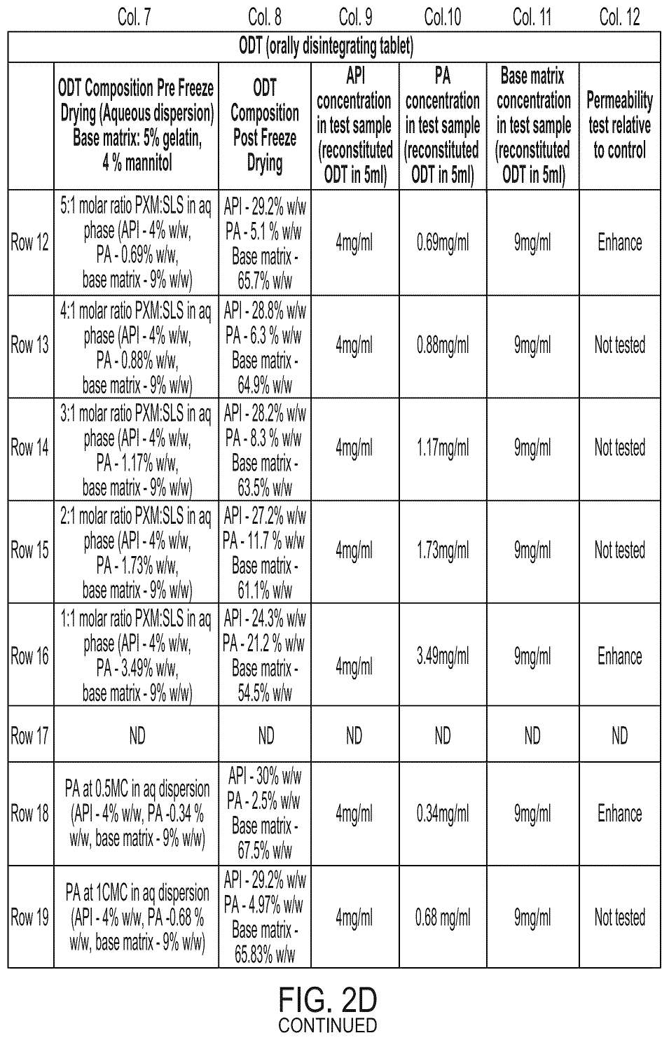

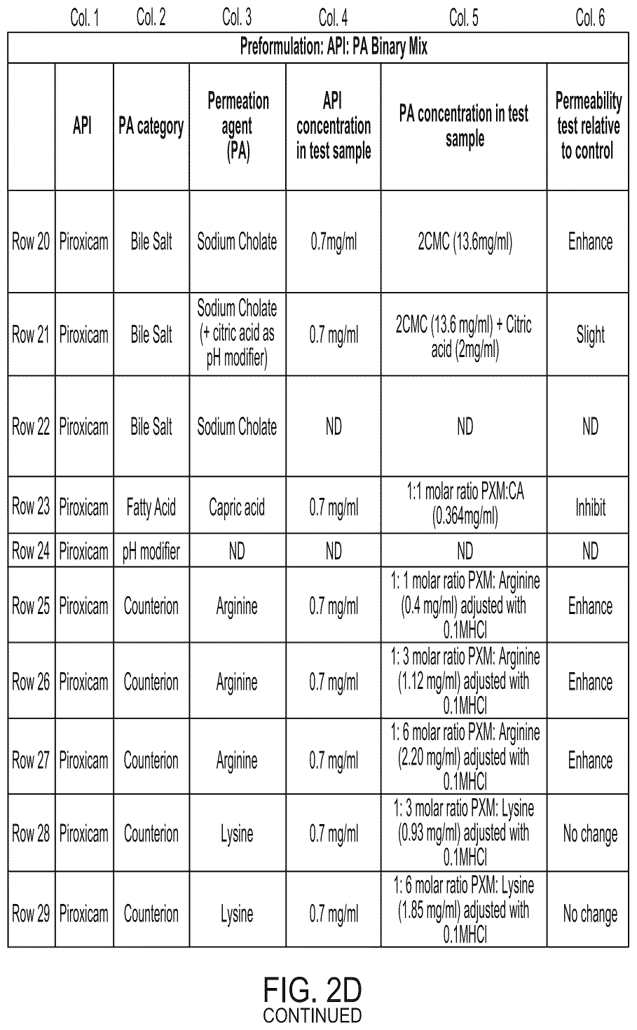

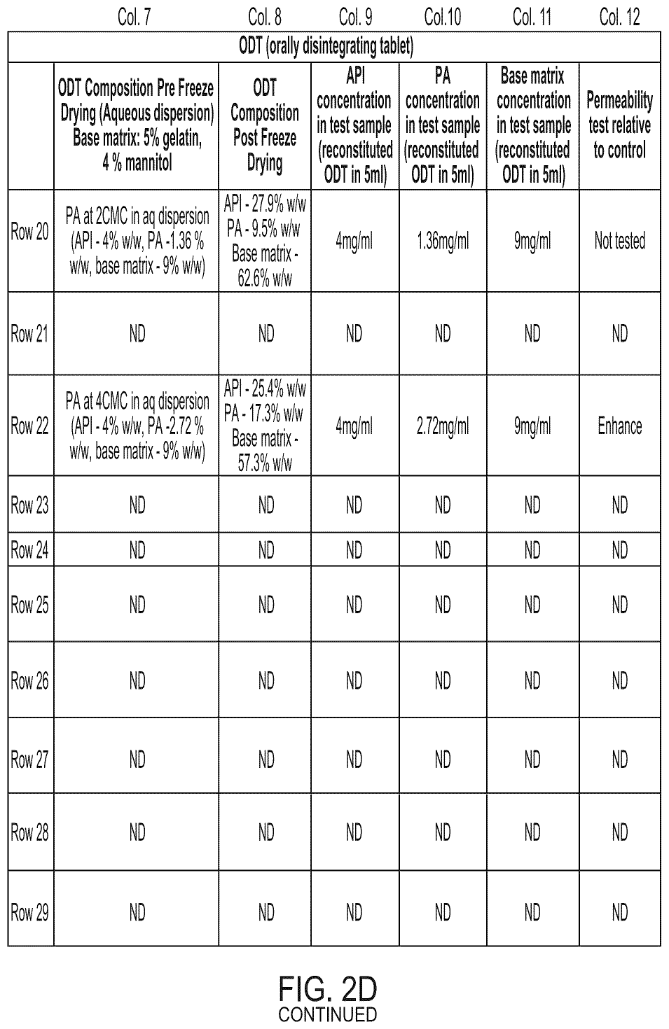

Before performing permeability studies on APIs using ODTs, permeation agents were screened in pre-formulation permeability studies on binary mixes of the permeation agents and APIs. The compositions of these pre-formulation binary mixtures can be shown in FIGS. 1A-1E and 2A-2D.

An appropriate aliquot (e.g., 0.5 ml) of the drug and permeation agent binary mixture was added to a donor chamber in the Transwell polymer insert. The donor chamber was then placed into a receiver chamber containing media. Samples were withdrawn periodically from the receiver chamber, and the concentration of drug in those samples was determined. Drug permeation was calculated with respect to the drug concentration in the donor chamber at the start of the experiment. The measured drug content detected in those samples can be presented as drug transport at a fixed point or as a temporal profile.

### Freeze-Dried ODT Formulation Procedures

Formulation permeability studies for Piroxicam was also carried out using ODT formulations including selected permeation agents. The pharmaceutical compositions (ODTs) were produced by a freeze-drying process. The compositions of these pharmaceutical formulations can be shown in FIGS. 1A-1B and 2D. The freeze-dried ODT includes a base matrix formulation to help provide structure to the final freeze-dried tablets. The base matrix formulation is comprised of a matrix former and a structural former. The matrix former provides the network structure that imparts strength and resilience to the freeze-dried tablet. Suitable matrix formers can include gelatin, starch, or some combination thereof. The structural former is used to provide structural robustness to the freeze-dried tablet. Suitable structural formers can include sugars, including but not limited to mannitol, dextrose, lactose, galactose, cyclodextrin, or some combination thereof. The base matrix formulation may also include additional agents or excipients, such as permeation agents.

The base matrix was prepared by dissolving the matrix former and structural former in water to form a pre-mix. Other suitable solvents may be used. The API, permeation agent(s), and additional excipients were incorporated into the pre-mix to form an aqueous dispersion of the API. Where required, a pH modifier was added. The aqueous API dispersion was then scaled up to a desired batch size with water, at which point it was ready for dosing into blister trays containing preformed pockets.

The aqueous API dispersion may alternatively be made by preparing a dry mix. In some embodiments, the API may be dry-mixed with the permeation agent(s), and the dry mixture may be incorporated into the pre-mix with the remaining excipients to form an aqueous dispersion of the API. In another embodiment, the API may be dry-mixed with the permeation agent(s), and the dry blend may then be mixed with a portion of the pre-mix to form an intermediate API suspension that may be incorporated into the remaining pre-mix with the remaining excipients to form an aqueous dispersion of the API. In another embodiment, the API may be dry-mixed with the permeation agent(s), and the dry blend may be mixed with an appropriate amount of water. A portion of pre-mix may be incorporated into the API mixture. Multiple pre-mix portions may then be added until all of the pre-mix has been added.

Once the aqueous API formulation was prepared, predetermined aliquots of about 150 to 1200 mg (wet fill weight) of the formulation were dosed into each pocket on the blister trays at about 10° C. to 30° C. (±5° C.). The dosed formulation was then frozen in the pocket of the preformed blisters (hereafter referred to as frozen units) by passing the dosed blister trays through a cryogenic chamber at a range of sub-zero temperatures. The freezing temperature and duration of freezing were varied to ensure that the dosed API formulation was frozen.

The frozen units were then collected and held in a freezer at a sub-zero temperature appropriate for the formulation prior to freeze-drying. Alternatively, the frozen unit may be annealed for a suitable period of time to crystallize the structural former. Structural former crystallization can provide the frozen units with the structural strength to prevent collapse during freeze-drying.

Following the frozen hold, the frozen units were freeze-dried into pharmaceutical compositions (e.g., tablets). The freeze-drying cycle was optimized for individual formulations. During the freeze-drying process, water is removed from the frozen units by sublimation, leaving porous freeze-dried units which can rapidly disintegrate when placed in the oral cavity. The freeze-dried tablets were then removed from the freeze-drier and inspected for any defects. The inspected tablets were then placed in a storage cabinet at a temperature and humidity-controlled environment ready to be sealed in their preformed blister trays. Blister trays were sealed by placing a lidding foil on the preformed trays. The preformed trays were then cut to form individual blisters containing the freeze-dried ODTs. Following ODT formulation, ODTs were then re-dispersed in distilled water. An appropriate aliquot (e.g., 0.5 ml) of the dispersed formulation was sampled for the permeability study.

### Permeability Assessment Procedures

TR146 buccal cells seeded on Transwell plates were grown in a growth media for 25 to 28 days. Before the permeability assessment began, the TEER of each Transwell insert in the same growth media was measured. The inserts then underwent two washes with Hanks Balance Salt Solution (HBSS), and the TEER was measured again. The HBSS buffer in the Transwell was then decanted.

Three types of samples were used in permeability testing: (a) an API control sample, (b) a binary mixture of a permeation agent and API in water, and (c) a reconstituted aqueous dispersion of the ODT comprising the permeation agent and API. For each test sample, the permeability experiment was done in triplicate. Each of the API control samples and each of the binary mixtures of API and permeation agents were prepared in 20 ml of distilled water. Each of the ODT samples was dispersed in 5 ml of distilled water.

For each type of sample, 0.5 ml of sample was added directly to each donor chamber of the Transwell. The donor chamber was transferred to a new receiving chamber at each of the following time points: after 1, 2, 5, 15, 20, and 30 minutes cumulatively. Each receiving chamber contained 1.5 ml of HBSS. The test was performed at 36° C. on an orbital plate shaker at 150 rpm. Once all measurements were taken, the donor chamber was washed again with HBSS, and the TEER was measured to ensure that the integrity of the cell line was still acceptable.

After the permeability test was complete, an appropriate amount of solvent was added to the receiving chamber containing 1.5 ml of HBSS buffer. The appropriate solvent depends on the type of sample in the receiving chamber (e.g., 0.25 ml methanol for Piroxicam (API), 2.5 ml methanol for Piroxicam ODT, 2.5 ml methanol for Carvedilol (API), 1.5 ml acetronitrile for Atenolol (API), 1.5 ml acetronitrile for Atenolol ODT, and 2.5 ml methanol for Famotidine (API)).

For the binary mixture of API and permeation agent, each receiving chamber was shaken at 150 rpm for a minimum of two hours at room temperature. The liquid sample in the receiving chamber was then filtered using a 0.45 μm filter and appropriately aliquoted for HPLC analysis.

For the ODT formulations, after adding the appropriate solvent to the receiving chamber, the liquid sample in each receiving chamber was transferred into separate vials and then sonicated for 15 to 20 minutes in a water bath. The liquid sample in each vial was then filtered using a 0.45 μm filter and appropriately aliquoted for HPLC analysis.

An HPLC sample assay for each sample was then determined. Based on the HPLC data, the cumulative concentration of permeated API at each time point was converted into a mass (in mcg) that was then used to calculate the percentage cumulative permeability at each time point according to the following equation:

$$\% cumulative permeability at each time point = \frac{cumulative mass at each time point ( mcg )}{tested dose ( mcg )} * 100$$

For a temporal profile (time dependent), the percentage cumulative permeability at each time point was plotted against the respective time points.

### Example 1: Permeability Assessment of Piroxicam

Piroxicam is a weakly acidic BCS Class II drug. It has a molecular weight of 331.4 g/mol and water solubility of 0.143 mg/ml. It exhibits a weakly acid pKa of 5.1 and a log P value of 2.2.

The permeability of Piroxicam was assessed in combination with the following permeation agents: Pluronic F127, Pluronic L44, sodium cholate hydrate, Tween 21, Span 80, sodium lauryl sulfate, Dodecyl-B-D-Maltoside, capric acid, arginine, and lysine.

For this assessment, control samples (1_{PXM} and 3_{PXM}) containing Piroxicam alone at 0.7 mg/ml, with and without pH adjustment respectively, and nineteen binary mixtures of Piroxicam and permeation agents were prepared in distilled water. Each mixture contained 0.7 mg/ml of Piroxicam. The concentrations of the permeation agents in the mixtures are summarized in Table 2. The samples were prepared at room temperature and stirred before they were sampled for testing. The pH of the mixtures were then measured, and the theoretical percent ionized and percent un-ionized of Piroxicam at the respective pH values were calculated using the Henderson-Hasselbach equation.

TABLE 2
Summary of Piroxicam & permeation agent binary mixture samples

| Sample | | Permeation Agent | | Calculated | Calculated |
|---|---|---|---|---|---|
| Reference | Sample name | & concn (mg/ml) | pH | % ionized | % unionized |
| 1_{PXM} | Control | None | 5.30 | 61.31 | 38.69 |
| | (drug only) | | | | |
| 3_{PXM} | Control (drug only), | None | 6.64 | 97.20 | 2.80 |
| | pH adjusted with | | | | |
| | 0.1M NaOH | | | | |
| 35_{PXM} | PXM-PF127 | PF127 | 5.59 | 75.55 | 24.45 |
| | (0.5 × CMC) | 0.25 mg/ml | | | |
| 36_{PXM} | PXM-PF127 | PF127 | 5.64 | 77.62 | 22.38 |
| | (1 × CMC) | 0.5 mg/ml | | | |
| 10_{PXM} | PXM-PF127 | PF127 | 5.40 | 66.61 | 33.39 |
| | (2 × CMC) | (1 mg/ml) | | | |
| 13_{PXM} | PXM-P44 | PL44 | 4.80 | 33.39 | 66.61 |
| | (2 × CMC) | (15.84 mg/ml) | | | |
| 20_{PXM} | PXM-NaCholate | Sodium Cholate | 6.63 | 97.13 | 2.87 |
| | (0.5 × CMC) | (3.4 mg/ml) | | | |
| 37_{PXM} | PXM-NaCholate | Sodium Cholate | 6.61 | 97.00 | 3.00 |
| | (1 × CMC) | (6.8 mg/ml) | | | |
| 21_{PXM} | PXM-NaCholate | Sodium Cholate | 6.66 | 97.32 | 2.68 |
| | (2 × CMC) | (13.6 mg/ml) | | | |
| 19_{PXM} | PXM-NaCholate | Sodium Cholate | 5.51 | 71.99 | 28.01 |
| | (2 × CMC) + | (13.6 mg/ml)/ | | | |
| | Citric acid | Citric acid | | | |
| | | (2 mg/ml) | | | |
| 23_{PXM} | PXM-Tween 21 | Tween 21 | 5.37 | 65.06 | 39.94 |
| | (1:1 molar ratio) | (1.19 mg/ml) | | | |
| 24_{PXM} | PXM-Span 80 | Span 80 | 4.92 | 39.78 | 60.22 |
| | (1:1 molar ratio) | (0.91 mg/ml) | | | |
| 25_{PXM} | PXM-Tween 21: | Tween 21:Span 80 | 5.17 | 54.02 | 45.98 |
| | Span 80 (5:1) | (5:1) | | | |
| | (1:1 molar ratio) | (0.95 mg/ml of | | | |
| | | Tween 21 and | | | |
| | | 0.19 mg/ml of | | | |
| | | Span 80) | | | |
| 26_{PXM} | PXM-SLS | SLS | 5.08 | 48.85 | 51.15 |
| | (1:1 molar ratio) | (0.609 mg/ml) | | | |
| 27_{PXM} | PXM-DDM | DDM | 4.88 | 37.60 | 62.40 |
| | (1:1 molar ratio) | (1.015 mg/ml) | | | |
| 28_{PXM} | PXM-Capric acid | Capric acid | 4.26 | 12.63 | 87.37 |
| | (1:1 molar ratio) | (0.364 mg/ml) | | | |
| 30_{PXM} | PXM-Arginine | Arginine | 6.92^{a} | 98.51 | 1.49 |
| | (1:1 molar ratio) | (0.4 mg/ml) | | | |
| | adjusted with | | | | |
| | 0.1M HCl | | | | |
| 31_{PXM} | PXM-Arginine | Arginine | 6.54^{a} | 96.50 | 3.50 |
| | (1:3 molar ratio) | (1.12 mg/ml) | | | |
| | adjusted with | | | | |
| | 0.1M HCl | | | | |
| 32_{PXM} | PXM-Arginine | Arginine | 6.67^{a} | 97.38 | 2.62 |
| | (1:6 molar ratio) | (2.20 mg/ml) | | | |
| | adjusted with | | | | |
| | 0.1M HCl | | | | |
| 33_{PXM} | PXM-Lysine | Lysine | 6.79^{a} | 98.00 | 2.00 |
| | (1:3 molar ratio) | (0.93 mg/ml) | | | |
| | adjusted with | | | | |
| | 0.1M HCl | | | | |
| 34_{PXM} | PXM-Lysine | Lysine | 6.76^{a} | 97.86 | 2.14 |
| | (1:6 molar ratio) | (1.85 mg/ml) | | | |
| | adjusted with | | | | |
| | 0.1M HCl | | | | |

^{a}pH after pH adjustment with 0.1M HCl

For the permeability assessment, the control samples and samples of the binary mixtures summarized in Table 2 were tested by the procedure described in the preceding Permeability Assessment Procedures section. For each test sample (done in triplicate), 0.5 ml containing 0.35 mg of Piroxicam was added to the donor chamber of the Transwell plate. Prior to starting the permeability assessment, 1.5 ml of HBSS was added to each receiving chamber. After completion of the permeability assessment, 0.25 ml of methanol was added to each receiving chamber. The Transwell plates were then placed on a plate shaker at 150 rpm for two hours at room temperature. The samples were then prepared for HPLC analysis and assayed for Piroxicam content.

To determine the drug concentration of Piroxicam in the samples, an HPLC method was developed and validated. The method was optimized with chromatographic conditions of 60/40 TFA/ACN (trifluoroacetic acid/acetonitrile), 0.1% TFA, an Eclipse Plus HPLC column (3.5μ C18, 150×4.6 mm), 10 μl injection volume, a flow rate of 1 ml/min at ambient temperature (25° C.) and detected at 330 nm (UV spectrometry). The linearity range is 0.19-100 mcg/ml of Piroxicam in methanol, acceptable linearity (R²=0.999), and low LOD and LOQ (0.10 mcg/ml and 0.30 mcg/ml respectively). The run time was 8 minutes.

The results of the percentage cumulative permeability of Piroxicam are summarized in Table 3.

TABLE 3
Pre-formulation permeability results of Piroxicam & permeation
agent binary mixtures - percentage cumulative permeability

| | | Permeation | | % Cumulative Permeability (n = 3) | | | | | |
|---|---|---|---|---|---|---|---|---|---|
| Sample | | Agent & concn | | 1 | 2 | 5 | 15 | 20 | 30 |
| Ref | Sample name | (mg/ml) | pH | min | min | min | min | min | min |
| 1_{PXM} | Control | None | 5.3 | 0.08 | 0.24 | 0.96 | 3.82 | 5.89 | 9.96 |
| | (drug only) | | | | | | | | |
| 3_{PXM} | Control (drug | None | 6.4 | 0.13 | 0.41 | 1.60 | 5.88 | 7.91 | 11.24 |
| | only), pH | | | | | | | | |
| | adjusted | | | | | | | | |
| 35_{PXM} | PXM-PF127 | PF127 | 5.59 | 0.26 | 3.00 | 9.91 | 31.99 | 38.74 | 48.15 |
| | (0.5 × CMC) | 0.25 mg/ml | | | | | | | |
| 36_{PXM} | PXM-PF127 | PF127 | 5.64 | 0.62 | 3.08 | 9.13 | 28.81 | 35.58 | 45.29 |
| | (1 × CMC) | 0.5 mg/ml | | | | | | | |
| 10_{PXM} | PXM-PF127 | PF127 | 5.4 | 0.29 | 0.95 | 4.39 | 13.94 | 19.15 | 27.22 |
| | (2 × CMC) | (1 mg/ml) | | | | | | | |
| 13_{PXM} | PXM-P44 | PL44 | 4.80 | 0.24 | 0.33 | 1.75 | 6.58 | 8.03 | 9.94 |
| | (2 × CMC) | (15.84 mg/ml) | | | | | | | |
| 20_{PXM} | PXM- | Sodium | 6.63 | 0.04 | 0.09 | 0.72 | 4.33 | 6.18 | 10.48 |
| | NaCholate | Cholate | | | | | | | |
| | (0.5 × CMC) | (3.4 mg/ml) | | | | | | | |
| 37_{PXM} | PXM- | Sodium | 6.61 | 0.42 | 1.39 | 5.40 | 20.35 | 26.53 | 39.52 |
| | NaCholate | Cholate | | | | | | | |
| | (1 × CMC) | (6.8 mg/ml) | | | | | | | |
| 21_{PXM} | PXM- | Sodium | 6.66 | 0.03 | 0.11 | 0.78 | 5.70 | 8.34 | 14.44 |
| | NaCholate | Cholate | | | | | | | |
| | (2 × CMC) | (13.6 mg/ml) | | | | | | | |
| 19_{PXM} | PXM- | Sodium | 5.51 | 0.14 | 0.40 | 1.42 | 5.27 | 6.78 | 9.39 |
| | NaCholate | Cholate | | | | | | | |
| | (2 × CMC) + | (13.6 mg/ml)/ | | | | | | | |
| | Citric acid | Citric acid | | | | | | | |
| | | (2 mg/ml) | | | | | | | |
| 23_{PXM} | PXM-Tween 21 | Tween 21 | 5.37 | 0.37 | 1.16 | 5.10 | 13.18 | 16.21 | 20.82 |
| | (1:1 molar ratio) | (1.19 mg/ml) | | | | | | | |
| 24_{PXM} | PXM-Span 80 | Span 80 | 4.92 | 0.12 | 0.26 | 0.68 | 1.51 | 1.86 | 2.40 |
| | (1:1 molar ratio) | (0.91 mg/ml) | | | | | | | |
| 25_{PXM} | PXM-Tween | Tween | 5.17 | 0.11 | 0.75 | 2.10 | 7.55 | 9.42 | 12.03 |
| | 21:Span 80 (5:1) | 21:Span 80 (5:1) | | | | | | | |
| | (1:1 molar ratio) | (0.95 mg/ml of | | | | | | | |
| | | Tween 21 and | | | | | | | |
| | | 0.19 mg/ml of | | | | | | | |
| | | Span 80) | | | | | | | |
| 26_{PXM} | PXM-SLS | SLS | 5.08 | 0.09 | 0.28 | 1.22 | 7.75 | 12.29 | 21.94 |
| | (1:1 molar ratio) | (0.609 mg/ml) | | | | | | | |
| 27_{PXM} | PXM-DDM | DDM | 4.88 | 0.14 | 0.65 | 2.43 | 9.22 | 15.11 | 25.60 |
| | (1:1 molar ratio) | (1.015 mg/ml) | | | | | | | |
| 28_{PXM} | PXM-Capric | Capric acid | 4.26 | 0.14 | 0.27 | 0.80 | 3.19 | 4.06 | 6.11 |
| | acid | (0.364 mg/ml) | | | | | | | |
| | (1:1 molar ratio) | | | | | | | | |
| 30_{PXM} | PXM-Arginine | Arginine | 6.92^{a} | 0.31 | 0.70 | 2.21 | 6.58 | 8.82 | 12.49 |
| | (1:1 molar ratio) | (0.4 mg/ml) | | | | | | | |
| | adjusted with | | | | | | | | |
| | 0.1MHCl | | | | | | | | |
| 31_{PXM} | PXM-Arginine | Arginine | 6.54^{a} | 0.32 | 0.73 | 2.00 | 5.62 | 7.82 | 11.66 |
| | (1:3 molar ratio) | (1.12 mg/ml) | | | | | | | |
| | adjusted with | | | | | | | | |
| | 0.1MHCl | | | | | | | | |
| 32_{PXM} | PXM-Arginine | Arginine | 6.67^{a} | 0.20 | 0.31 | 1.34 | 4.33 | 6.40 | 10.15 |
| | (1:6 molar ratio) | (2.20 mg/ml) | | | | | | | |
| | adjusted with | | | | | | | | |
| | 0.1MHCl | | | | | | | | |
| 33_{PXM} | PXM-Lysine | Lysine | 6.79^{a} | 0.21 | 0.44 | 1.28 | 3.96 | 5.80 | 8.52 |
| | (1:3 molar ratio) | (0.93 mg/ml) | | | | | | | |
| | adjusted with | | | | | | | | |
| | 0.1MHCl | | | | | | | | |
| 34_{PXM} | PXM-Lysine | Lysine | 6.76^{a} | 0.18 | 0.51 | 1.47 | 4.18 | 6.10 | 9.45 |
| | (1:6 molar ratio) | (1.85 mg/ml) | | | | | | | |
| | adjusted with | | | | | | | | |
| | 0.1MHCl | | | | | | | | |

^{a}pH after pH adjustment with 0.1M HCl

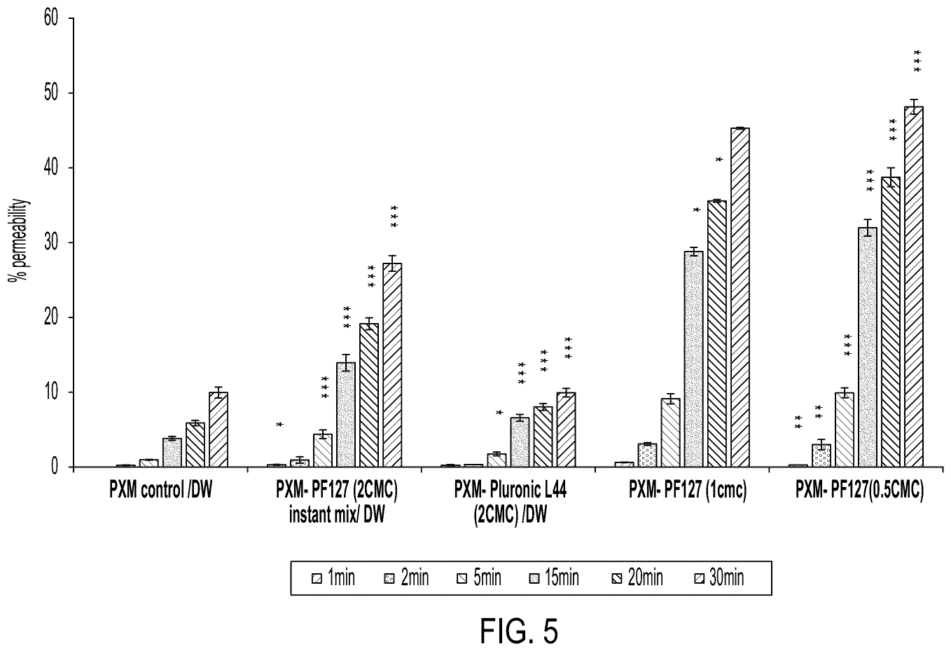

FIG. 5 depicts exemplary permeability data illustrating the effect of using polymeric non-ionic surfactants as permeability enhancers for Piroxicam. Pluronic F127 (PF127) and Pluronic LA4 (PLA4) were used as model surfactants. PF127 and PL44 molecular weights are 12600 g/mol and 2200 g/mol, respectively. Each grade has a different hydrophilic-lipophilic balance (HLB) value; the HLB values of PF127 and PL44 are 22 and 16, respectively. Both grades were investigated at a concentration of 2×CMC. PF127 was also assessed at 0.5×CMC and ×1 CMC. All samples were prepared by mixing the surfactant solution with Piroxicam suspension for one minute before carrying out the permeability assessment. The results show that the solubilization and micellization effects of PL44 and PF127 increase the permeability of Piroxicam.

In the case of PL44, the permeability of Piroxicam increased slightly when PL44 was added, with a maximum of a 1.7-fold increase and a 1.3-fold increase after 15 and 20 minutes, respectively. However, no difference was found in the percentage cumulative permeability compared to the control samples at 30 minutes. In the case of PF127, the addition of PF127 significantly increased the permeability of Piroxicam compared with the control sample starting at the first minute. At 0.5×CMC and 1×CMC, the permeability increased rapidly, ranging from a 7 to 12-fold increase in the first 15 minutes followed by a gradual decrease to approximately a 4.5-fold increase at 30 minutes, resulting in a percentage cumulative permeability of 48.15% and 45.29% at 30 minutes, respectively. At 2×CMC, the increase was more than 3-fold at all time points (p<0.001), resulting in a percentage cumulative permeability of 27.22% at 30 minutes. The difference in the permeability effect of the two grades can be attributed to the difference in the lengths of the PPO and PEO polymer blockers. For PF127, the lower permeability at 2×CMC could be due to more encapsulation of the API within micelles, which may decrease the permeability.

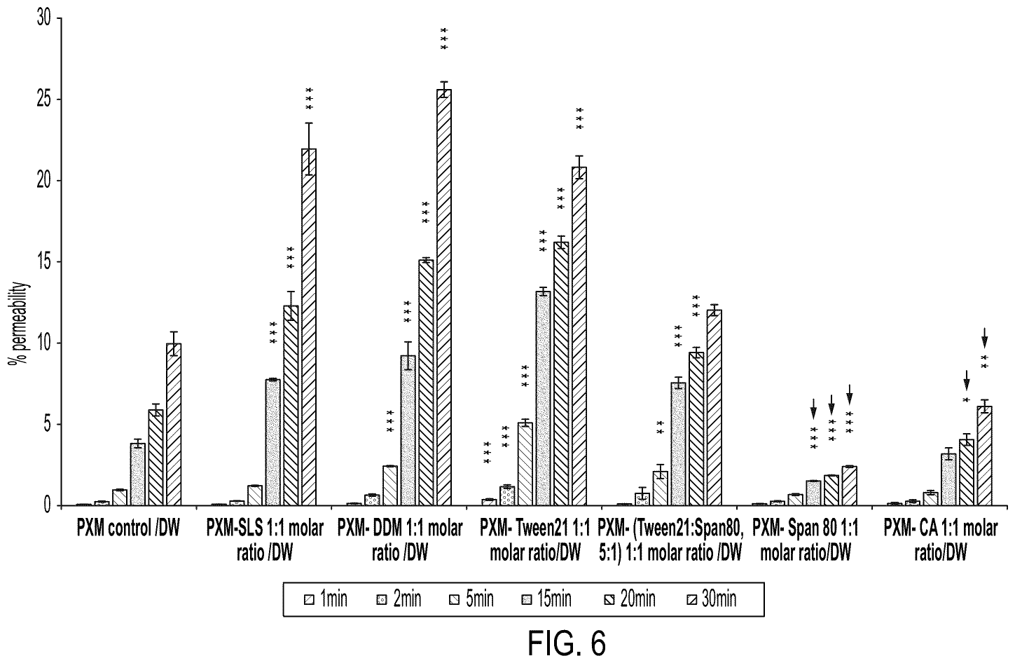

FIG. 6 depicts exemplary permeability data illustrating the effect of using various non-ionic and ionic surfactants and fatty acids as permeability enhancers for Piroxicam. Tween 21, Span 80, and a blend of both surfactants in a 5:1 ratio of Tween 21 to Span 80, with HLB values of 4.3, 13.3, and 11.8, respectively, were used as model non-ionic surfactants. Sodium lauryl sulfate (SLS), which has an HLB value of 40, was used as an anionic surfactant. Dodecyl-B-D-Maltoside (DDM), which has an HLB value of 13.5, was used as a glycosidic surfactant. All binary mixtures were screened at a 1:1 molar ratio to Piroxicam. All samples were prepared by mixing a surfactant solution with a Piroxicam suspension and then left to stir for 1 to 1.5 hours before carrying out the permeability assessment.

Span 80 is a non-polar, lipophilic surfactant with a low HLB of 4.3. It was hypothesized that Span 80 mainly disturbs the tissue integrity by forming hydrogen bonds with the polar head groups of the membrane lipids. However, in this experiment, Span 80 was observed to reduce the Piroxicam permeability significantly. Since Piroxicam was partially ionized at the pH of the sample (4.2), it may have displaced the polar heads of the membrane lipids and interacted with the Span 80. In addition, Span 80 can form lipophilic aggregates. Since Piroxicam is lipophilic in nature, the lipophilic aggregates may have delayed its release.

In contrast, the permeability of Piroxicam was increased when hydrophilic surfactants (SLS, Tween 21, and DDM) and a 5:1 blend of Tween 21 to Span 80 were used, all of which have HLB values greater than 10. Surfactants with HLB values in the range of 10-20 may enhance drug permeation by inserting themselves between the lipophilic tails of the bilayers, thus disturbing the lipid arrangements in the cell membrane.

Tween 21, which has an HLB value of 13.3, achieved a 3.5 to 5-fold increase in permeability compared to Piroxicam control sample over the first 15 minutes and reached a percentage cumulative permeability of 20.82% (a 2.1-fold increase over the control sample) at 30 minutes. The blend of Tween 21 and Span 80, which has an HLB value of 11.8, also enhanced Piroxicam permeability compared to the control, with a 1.4 to 3-fold increase in permeability within the first 15 minutes and a percentage cumulative permeability of 12.03% (a 1.2-fold increase) at 30 minutes.

DDM, a glycosidic surfactant, has an HLB value of 13.5. DDM showed a statistically significant improvement in Piroxicam permeability, with a 1.75 to 2.6-fold increase in percentage cumulative permeability. It also showed the highest percentage cumulative permeability of all surfactants at 25.60% (a 2.6-fold increase over the control sample) after 30 minutes.

SLS has an HLB value of 40. An increase in Piroxicam permeability was only observed after 15 minutes, and a maximum of a 2.2-fold increase in percentage cumulative permeability compared to the control sample was observed at the 30 minute mark. Although SLS had the highest HLB value of surfactants tested, the enhancement effect on Piroxicam permeability was statistically significantly lower than the effect of Tween 21 and DDM. The lower permeability can be explained by the micellar lipid extraction effect of SLS and encapsulation in micelles.

FIG. 6 also shows the effect of fatty acids on the permeability of Piroxicam in comparison with the Piroxicam control sample. Capric acid (CA) was used as a medium-chain fatty acid (MCFA) model, with a C-10 carbon chain length. To prepare samples using CA, the required weight of CA was melted at 50° C. and then cooled to 25° C. to 30° C. Once cooled, but before solidified, the CA was mixed with a Piroxicam-water mixture and left to stir at room temperature until starting the permeability assessment.

Fatty acids were hypothesized to promote drug permeability by several processes such as drug solubilization, increasing membrane fluidity, and paracellular transport. It was also hypothesized that saturated fatty acids, especially those with a medium chain length (i.e., C10 or C12), display optimum activity when used for delivery through the buccal mucosa, which has a high content of polar lipids. However, in this experiment, the opposite effect was observed for CA and Piroxicam. CA significantly reduced Piroxicam permeability after 20 and 30 minutes when compared to the Piroxicam control. In the aqueous solution, fatty acid molecules above CMC may have self-assembled or aggregated to form micelles of various sizes and structures. Those micelles may have interfered with the efficiency of CA as a transient permeability enhancer and reduced drug permeability. However, the aggregation behavior and CMC value of different MCFAs are highly dependent on various system properties such as pH, temperature, and ionic strength. Additionally, the lipophilic fatty acid aggregates may delay the release of lipophilic drugs, and Piroxicam is a lipophilic drug, with a log P value of 2.2.

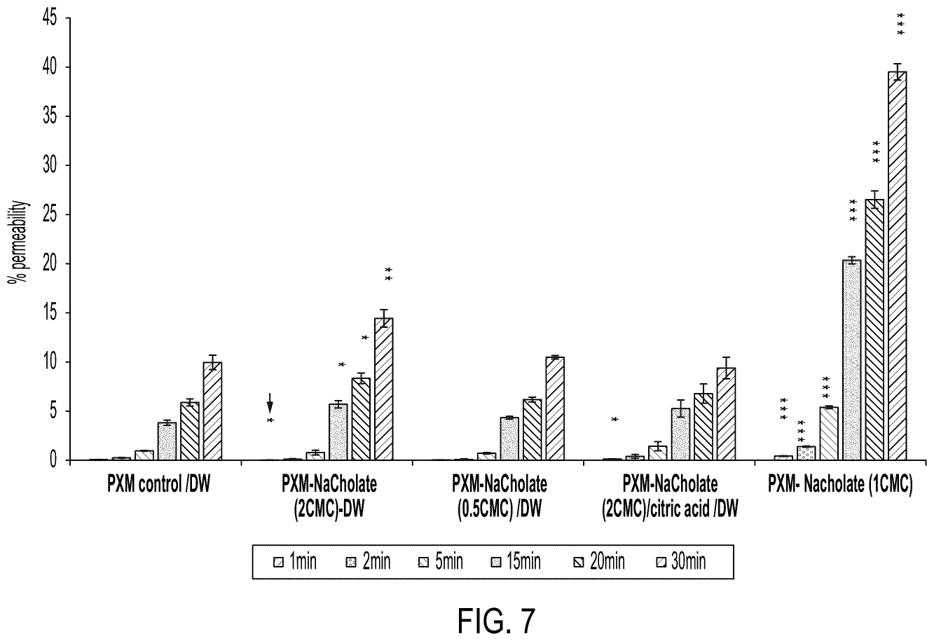

FIG. 7 depicts exemplary permeability data illustrating the effect of using bile salts as permeability enhancers for Piroxicam. Sodium cholate hydrate was used as a model bile salt, and its effect on Piroxicam permeability was assessed at 0.5 CMC, 1 CMC, and 2 CMC. The samples had high pH values compared to the control, with pH values of 6.63, 6.61, and 6.66, respectively. A 2 CMC sample that was pH-adjusted to 5.51 using citric acid was also assessed.

Samples prepared using 0.5 CMC showed a slight, statistically non-significant increase in Piroxicam permeability compared to the control. At 1 CMC, the permeability was significantly increased 5 to 6-fold for the first 15 minutes. A percentage cumulative permeability of 39.52% (a 4-fold increase over the control) was observed at 30 minutes. At 2 CMC, there was a small but statistically significant 1.5-fold increase in the percentage cumulative permeability at the first minute. Percentage cumulative permeability reached 14.44% after 30 minutes. For the 2 CMC sample that was pH-adjusted using citric acid, the increase in permeability was slight and statistically non-significant. These results may be explained by the dual effect of sodium cholate. It may disrupt the lipid bilayer packing and enhance Piroxicam solubility, and consequently its permeability, by micellization. When citric acid is added, although the percentage of unionized drug increases (favoring an increase in permeability), the drug solubility may decrease, which may explain the lower permeability of the sample containing citric acid.

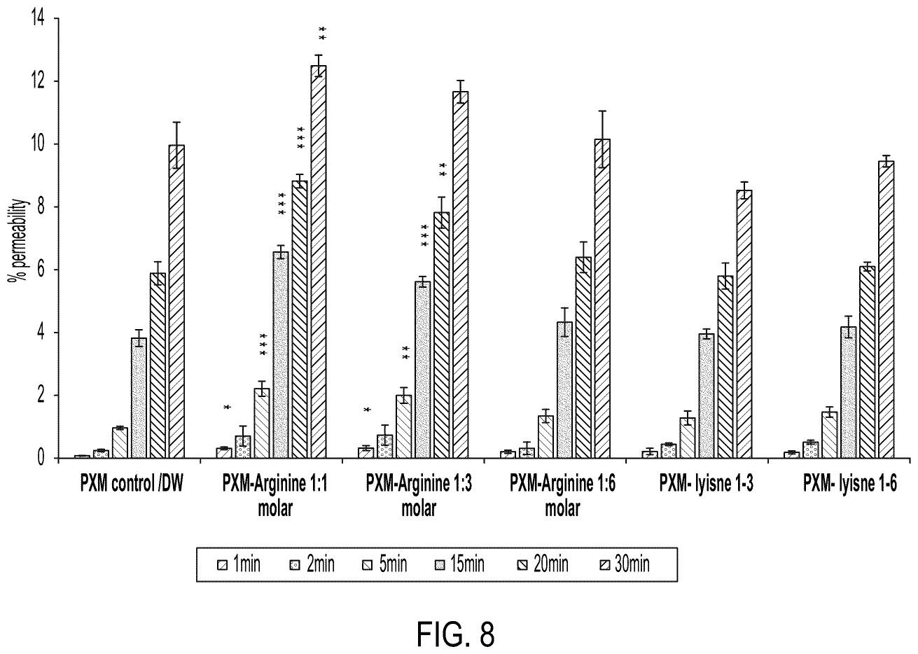

FIG. 8 depicts exemplary permeability data illustrating the effect of using various counterions as permeability enhancers for Piroxicam. Since Piroxicam is a weak acid (pKa=5.1), basic amino acids such as arginine and lysine were used as counterions. Both arginine and lysine have 2 basic groups: pKa1=9.04 and pKa2=12.48 for arginine, while pKa1=8.95 and pKa2=10.53 for lysine. To ensure that both the Piroxicam and counterions were fully ionized, all of the samples were prepared at a pH 2 units different from their pKas. Therefore, the pH of the samples was adjusted to the range of 6.5-7 using 0.1M HCl.

Arginine was studied at 1:1, 1:3, and 1:6 molar ratios of Piroxicam to arginine. Significant increases in percentage cumulative permeability were observed at both the 1:1 and 1:3 molar ratios, mainly at the early time points (over 1-5 minutes) with 2 to 4-fold increases in % cumulative permeability. At the later time points, from 15 to 30 minutes, the increase was less pronounced and decreased from 1.7-fold to 1.2-fold over this time period. For the 1:6 molar ratio sample, no increase in permeability was observed. Amino acids as counterions may reduce the lipophilicity of the drug, thus decreasing the drug's log P value as the fraction of amino acid increases. Therefore, increasing the ratio of counterion to API may not increase drug permeability.

Lysine was studied at 1:3 and 1:6 molar ratios of Piroxicam to lysine. The results showed no significant enhancement in Piroxicam permeability. This may be due to the lower difference in pKa (ΔpKa) values between Piroxicam and lysine (ΔpKa≥5) compared to Piroxicam and arginine (ΔpKa>7). Arginine also has more H-bonds than lysine and may form stronger ionic bonds with weak acidic API such as Piroxicam.

In addition to the binary mixtures of API and permeation agent, freeze-dried ODTs comprising Piroxicam and a selection of permeation agents, namely PF127, sodium cholate hydrate, and SLS, were manufactured for the permeability assessment. Batches of ODTs with different concentrations of permeation agents were manufactured. The ODTs were manufactured using the procedures described in the Freeze-dried ODT Formulation Procedures section.

An aqueous dispersion of the ODT formulation included a base matrix (comprising a matrix former (e.g., gelatin) and a structure former (e.g., mannitol)) and Piroxicam. Batches were prepared with and without permeation agents. A wet fill weight of 500 mg of the aqueous dispersion was dosed into each preformed pocket of a blister pack. The aqueous dispersion contained 4% w/w of Piroxicam, giving rise to a dose of 20 mg Piroxicam per tablet.

A selection of these batches was assessed for Piroxicam permeability. The composition of the ODT formulations (as aqueous dispersions prior to freeze-drying and as freeze-dried tablets) of these batches are summarized in Table 4.

TABLE 4
Summary of Piroxicam ODT batches for permeability study

| | | Formulation composition | | |
|---|---|---|---|---|
| | | | Pre freeze drying | Post freeze drying |
| Batch Ref. | Purpose of batch | Component | (% w/w) | (% w/w) |
| Study 1 | Control | Piroxicam | 4.00 | 30.77 |
| PXM: Batch 1 | | Base Matrix | 9.00 | 69.23 |
| | | Water | gs 100.00 | — |
| Study 1 | PF 127 at | Piroxicam | 4.00 | 30.53 |
| PXM: Batch 2 | 2× CMC in | PF127 | 0.10 | 00.76 |
| | aqueous dispersion | Base Martix | 9.00 | 68.70 |
| | (0.1% w/w) | Water | gs 100.00 | — |
| Study 1 | PF 127 at | Piroxicam | 4.00 | 28.57 |
| PXM: Batch 5 | 20× CMC in | PF127 | 1.00 | 07.1 |
| | aqueous dispersion | Base Matrix | 9.00 | 64.29 |
| | (1.0% w/w) | Water | qs 100.00 | — |
| Study 1 | PF 127 at | Piroxicam | 4.00 | 27.59 |
| PXM: Batch 6 | 30× CMC in | P127 | 1.50 | 10.34 |
| | aqueous dispersion | Base Matrix | 9.00 | 62.07 |
| | (1.5% w/w) | Water | qs 100.00 | — |
| Study 2 | Sodium Cholate at | Piroxicam | 4.00 | 29.99 |
| PXM: Batch 1 | 0.5× CMC in | NaCholate | 0.34 | 2.55 |
| | aqueous dispersion | Base Matrix | 9.00 | 67.47 |
| | (0.34% w/w) | Water | gs 100.00 | — |
| Study 2 | Sodium Cholate at | Piroxicam | 4.00 | 25.45 |
| PXM: Batch 4 | 4 × CMC in | NaCholate | 2.72 | 17.30 |
| | aqueous dispersion | Base Matrix | 9.00 | 57.25 |
| | (2.72% w/w) | Water | gs 100.00 | — |
| Study 3 | SLS at 5:1 molar | Piroxicam | 4.00 | 29.22 |
| PXM: Batch 1 | ratio PXM:SDS | SLS | 0.69 | 5.04 |
| | (0.69% w/w) | Base Matrix | 9.00 | 65.74 |
| | | Water | qs 100.00 | — |
| Study 3 | SLS at 1:1 molar | Piroxicam | 4.00 | 24.26 |
| PXM: Batch 5 | ratio PXM:SDS | SLS | 3.49 | 21.16 |
| | (3.49% w/w) | Base Matrix | 9.00 | 54.57 |
| | | Water | gs 100.00 | — |

Footnote:
The concentration for base matrix (pre freeze drying) can range from about 7% to 15%

To determine the concentration of Piroxicam in the ODT samples, the same HPLC method used to determine the concentration of Piroxicam in the API-only and API-permeation agent mixtures was used. As before, the method was optimized with chromatographic conditions of 60/40 TFA/ACN (trifluoroacetic acid/acetonitrile), 0.1% TFA, an Eclipse Plus HPLC column (3.5μ C18, 150×4.6 mm), 10 μl injection volume, a flow rate of 1 ml/min at ambient temperature (25° C.) and detected at 330 nm (UV spectrometry). The linearity range is 0.19-100 mcg/ml of Piroxicam in methanol, acceptable linearity (R²=0.999), and low LOD and LOQ (0.10 mcg/ml and 0.30 mcg/ml respectively). The run time was 8 minutes.

Prior to the permeability assessment, a freeze-dried ODT from each batch, each ODT containing a target dose of 20 mg Piroxicam, was dissolved in 5 ml of distilled water (i.e., 4 mg/ml Piroxicam) and stirred for five minutes on a magnetic stirrer. The pH was then measured to ensure there was no drastic change in pH, which could result in a different permeability due to the ratio of ionized to unionized molecules.

The permeability assessment was performed pursuant to the process described in the Permeability Assessment Procedures section. The results of the percentage cumulative permeability of Piroxicam are summarized in Table 5.

TABLE 5
Permeability results of Piroxicam freeze dried
ODT - Percentage Cumulative Permeability

| | Purpose of batch | | % Cumulative Permeability (n = 3) | | | | | |
|---|---|---|---|---|---|---|---|---|
| | (Permeation agent | | 1 | 2 | 5 | 15 | 20 | 30 |
| Batch Ref. | concentration) | pH | min | min | min | min | min | min |
| Study 1 | Control | 5.89 | 0.04 | 0.19 | 0.76 | 2.55 | 3.34 | 5.14 |
| PXM: Batch 1 | | | | | | | | |
| Study 1 | PF 127 at 2 × CMC | 5.81 | 0.08 | 0.31 | 0.99 | 3.22 | 4.65 | 7.28 |
| PXM: Batch 2 | in aqueous | | | | | | | |
| | dispersion | | | | | | | |
| | (0.1% w/w) | | | | | | | |
| Study 1 | PF 127 at | 5.52 | 0.08 | 0.29 | 0.93 | 3.08 | 4.33 | 7.01 |
| PXM: Batch 5 | 20 × CMC in | | | | | | | |
| | aqueous dispersion | | | | | | | |
| | (1% w/w) | | | | | | | |
| Study 1 | PF 127 at | 5.47 | 0.11 | 0.34 | 1.05 | 3.5 | 5.22 | 8.27 |
| PXM: Batch 6 | 30 × CMC in | | | | | | | |
| | aqueous dispersion | | | | | | | |
| | (1.5% w/w) | | | | | | | |
| Study 2 | Sodium Cholate at | 5.9 | 0.07 | 0.29 | 1.07 | 3.44 | 4.46 | 6.64 |
| PXM: Batch 1 | 0.5 × CMC in | | | | | | | |
| | aqueous dispersion | | | | | | | |
| | (0.34% w/w) | | | | | | | |
| Study 2 | Sodium Cholate at | 6.1 | 0.06 | 0.26 | 0.82 | 2.96 | 3.85 | 5.53 |
| PXM: Batch 4 | 4 × CMC in | | | | | | | |
| | aqueous dispersion | | | | | | | |
| | (2.72% w/w) | | | | | | | |
| Study 3 | SLS at 5:1 molar | 6.1 | 0.06 | 0.21 | 0.73 | 2.89 | 3.95 | 6.41 |
| PXM: Batch 1 | ratio PXM:SDS | | | | | | | |
| | (0.69% w/w) | | | | | | | |
| Study 3 | SLS at 1:1 molar | 6.4 | 0.11 | 0.33 | 0.95 | 3.38 | 4.69 | 7.19 |
| PXM: Batch 5 | ratio PXM:SDS | | | | | | | |
| | (3.49% w/w) | | | | | | | |

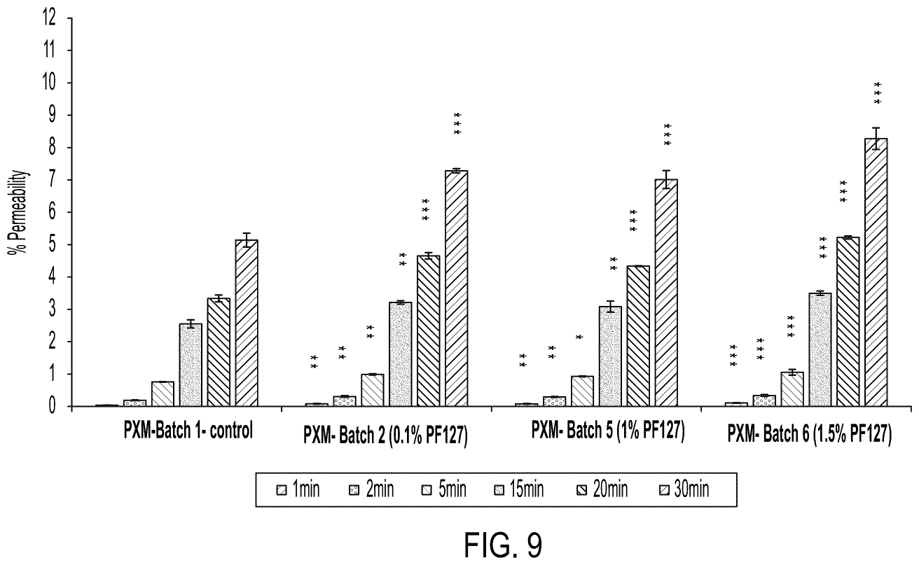

FIG. 9 depicts exemplary permeability data illustrating the effect of using a non-ionic surfactant as a permeability enhancer for Piroxicam reconstituted in a freeze-dried orally disintegrating tablet. PF127 was used as a model surfactant. Its effect on the permeability of Piroxicam in the freeze-dried ODT was assessed at concentrations of 0.1% w/w, 1% w/w, and 1.5% w/w in the aqueous dispersion. These concentrations equate to 2×CMC, 20×CMC, and 30×CMC, respectively. PF127 can form micelles in an aqueous environment when its concentration is at or above the CMC, and the micelles may enhance the permeation of Piroxicam by solubilization and encapsulation within the micelles. In this experiment, the permeation of Piroxicam for the freeze-dried ODT tablets was enhanced in the presence of PF127. The percentage cumulative permeation of the three batches of tablets was comparable, with each showing a 1.3 to 2.3-fold increase in permeation. At these concentrations, sufficient solubilization and/or micellization may have occurred such that similar amounts of micelles carrying Piroxicam were formed and permeated across the buccal cell line.

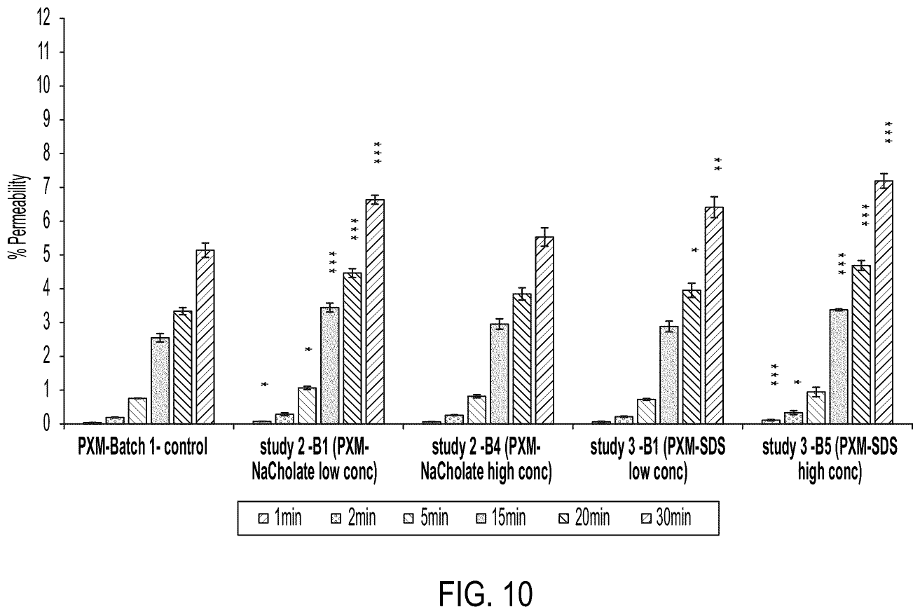

FIG. 10 depicts exemplary permeability data illustrating the effect of using bile salts and anionic surfactants as permeability enhancers for Piroxicam reconstituted in a freeze-dried orally disintegrating tablet. Sodium cholate hydrate was used as a model bile salt. Its effect on the permeability of Piroxicam in the freeze-dried ODT was assessed at concentrations of 0.34% w/w and 2.72% w/w in the aqueous dispersion, which equate to 0.5×CMC and 4×CMC, respectively. At 0.5×CMC, permeability of Piroxicam was only slightly increased. At 4×CMC, permeability was reduced, and percentage cumulative permeability was similar to the control sample. The difference between the two concentrations may be due to the size of the micelles. At CMC, sodium cholate micelles are primarily present as dimers. At concentrations exceeding CMC, the dimers can interact to form larger secondary structures, which may be more difficult to permeate.

FIG. 10 also shows the effect of SLS on the permeability of the Piroxicam ODT compared with the Piroxicam control sample. Its effect was assessed at concentrations of 0.69% w/w and 3.49% w/w in the aqueous dispersion, which equate to PXM: SLS molar ratios of 5:1 and 1:1, respectively. Permeability of the Piroxicam ODT was increased in the presence of SLS. The 3.49% w/w SLS sample showed a marginally higher but statistically significant increase in permeability compared to the 0.69% w/w SLS sample. The increase in percentage cumulative permeation was small in the first 5 minutes and was followed by a greater increase in permeability from 15 minutes onward. The binary mixture containing a 1:1 molar ratio of PXM:SLS exhibited a similar trend in the temporal profile.

### Example 2: Permeability Assessment of Carvedilol

Carvedilol (CAV) is a weakly basic BCS Class II API. It has a molecular weight of 406.5 g/mol, a log P value of 3.05, a basic pKa1 of 7.8, and an acidic pKa2 of 15. Carvedilol exhibits pH-dependent solubility, with lower solubility and ionization at high pH values and higher solubility and ionization at lower pH values. Its water solubility is 4.44 μg/ml.

The permeability of Carvedilol was assessed in combination with the following permeation agents: PF127, sodium cholate hydrate, Tween 21, Span 80, SLS, DDM, capric acid, citric acid, aspartic acid, and alanine.

For this assessment, a control sample (1_{CAV}) containing Carvedilol alone and fifteen binary mixtures of Carvedilol and permeation agents, each mixture containing 1.74 mg/ml of Carvedilol, were prepared in water. The concentrations of the permeation agents in the mixtures are summarized in Table 6. The samples were prepared at room temperature and stirred before they were sampled for testing. The pH of the mixtures were then measured, and the theoretical percent ionized and percent un-ionized of Carvedilol at the respective pH values were calculated using the Henderson-Hasselbach equation.

TABLE 6
Summary of Carvedilol: Permeation Agent binary mix samples

| Sample | | Permeation agent & | | Calculated | Calculated |
|---|---|---|---|---|---|
| Reference | Sample name | concn (mg/ml) | pH | % ionised | % unionised |
| 1_{CAV} | Control (drug only) | N/A | 7 | 86.32 | 13.68 |
| 5_{CAV} | CAV- PF127 | PF127 | 6.11 | 98.00 | 2.00 |
| | (2 × CMC) | (1 mg/ml) | | | |
| 6_{CAV} | CAV- PF127 | PF127 | 4.01 | 99.98 | 0.02 |
| | (2 × CMC)-citric | (1 mg/ml) | | | |
| | acid | Citric acid | | | |
| | | (0.5 mg/ml) | | | |
| 7_{CAV} | CAV- NaCholate | NaCholate | 7.55 | 64.01 | 35.99 |
| | (2 × CMC) | (13.46 mg/ml) | | | |
| 8_{CAV} | CAV- NaCholate | NaCholate | 4.51 | 99.95 | 0.05 |
| | (2 × CMC)-citric | (13.46 mg/ml) | | | |
| | acid | Citric acid (4 mg/ml) | | | |
| 9_{CAV} | CAV- SLS | SLS | 9.27 | 3.28 | 96.72 |
| | (1:1 molar ratio) | (1.21 mg/ml) | | | |
| 10_{CAV} | CAV- DDM | DDM | 7.55 | 64.01 | 35.99 |
| | (1:1 molar ratio) | (2.07 mg/ml) | | | |
| 11_{CAV} | CAV-Tween21 | Tween 21 | 7.2 | 79.92 | 20.08 |
| | (1:1 molar ratio) | (2.41 mg/ml) | | | |
| 12_{CAV} | CAV- capric acid | Capric acid | 5.84 | 98.92 | 1.08 |
| | (1:2 molar ratio) | (1.5 mg/ml) | | | |
| 13_{CAV} | CAV- Citric acid | Citric acid | 5.84 | 98.76 | 1.24 |
| | (0.05 mg/ml) | (0.05 mg/ml) | | | |
| 14_{CAV} | CAV- Citric acid | Citric acid | 4.2 | 99.97 | 0.03 |
| | (0.5 mg/ml) | (0.5 mg/ml) | | | |
| 15_{CAV} | CAV- Aspartic acid | Aspartic acid | 4.8 | 99.90 | 0.10 |
| | (1:1 molar ratio) | (0.58 mg/ml) | | | |
| 16_{CAV} | CAV- Aspartic acid | Aspartic acid | 3.97^{a} | 99.99 | 0.01 |
| | (1:3 molar ratio) | (1.74 mg/ml) | | | |
| | (adjusted with | | | | |
| | 0.1M NaOH) | | | | |
| 17_{CAV} | CAV-Aspartic acid | Aspartic acid | 4.05^{a} | 99.98 | 0.02 |
| | (1:6 molar ratio) | (3.48 mg/ml) | | | |
| | (adjusted with | | | | |
| | 0.1M NaOH) | | | | |
| 18_{CAV} | CAV- Alanine | Alanine | 4.83^{b} | 99.89 | 0.11 |
| | (1:1 molar ratio) | (0.4 mg/ml) | | | |
| | (adjusted with | | | | |
| | 0.1M HCL) | | | | |
| 19_{CAV} | CAV-Span 80 | Span 80 | 7.77 | 51.73 | 48.27 |
| | (1:1 molar ratio) | (1.86 mg/ml) | | | |

^{a}pH after pH adjustment with 0.1M NaOH
^{b}pH after pH adjustment with 0.1M HCL

For the permeability assessment, the control samples and samples of the binary mixtures summarized in Table 6 were tested by the procedure described in the Permeability Assessment Procedures section. For each test sample (done in triplicate), 0.5 ml containing 0.87 mg of Carvedilol was added to the donor chamber of the Transwell plate. Prior to starting the permeability assessment, 1.5 ml of HBSS was added to each receiving chamber. After completion of the permeability assessment, 2.5 ml of methanol was added to each receiving chamber. The Transwell plates were then placed on a plate shaker at 150 rpm for two hours at room temperature. The samples were then prepared for HPLC analysis and assayed for Carvedilol content.

To determine the drug concentration of Carvedilol in the samples, an HPLC method was developed and validated. The method was optimized with chromatographic conditions of 55/45 v/v TFA/ACN (trifluoroacetic acid; acetonitrile), 0.1% TFA, an Eclipse Plus HPLC column (3.5μ C18, 150×4.6 mm), 20 μl injection volume, a flow rate of 1 ml/min at ambient temperature (25° C.) and detected at 240 nm (UV spectrometry). The linearity range is 0.19-100 mcg/ml of Carvedilol in a mixture of methanol:HBSS in a 2:1 ratio, acceptable linearity (R²=0.9999), and low LOD and LOQ (0.014 mcg/ml and 0.042 mcg/ml respectively). The run time was 7 minutes.

The results of the percentage cumulative permeability of Carvedilol are summarized in Table 7.

TABLE 7
Pre-formulation permeability results of Carvedilol/Permeation
Agent binary mixes - Percentage Cumulative Permeability

| | | Permeation | | % Cumulative Permeability (n = 3) | | | | | |
|---|---|---|---|---|---|---|---|---|---|
| Sample | | agent & concn | | 1 | 2 | 5 | 15 | 20 | 30 |
| Ref | Sample name | (mg/ml) | pH | min | min | min | min | min | min |
| 1_{CAV} | Control (drug only) | N/A | 7 | 0.02 | 0.03 | 0.03 | 0.10 | 0.13 | 0.18 |
| 5_{CAV} | CAV- PF127 | PF127 | 6.11 | 0.00 | 0.00 | 0.03 | 0.13 | 0.59 | 0.70 |
| | (2 × CMC) | (1 mg/ml) | | | | | | | |
| 6_{CAV} | CAV- PF127 | PF127 | 4.01 | 0.06 | 0.17 | 0.59 | 2.98 | 3.78 | 5.62 |
| | (2 × CMC)-citric | (1 mg/ml) | | | | | | | |
| | acid | Citric acid | | | | | | | |
| | | (0.5 mg/ml) | | | | | | | |
| 7_{CAV} | CAV- NaCholate | NaCholate | 7.55 | 0.01 | 0.02 | 0.05 | 0.17 | 0.22 | 0.34 |
| | (2 × CMC) | (13.46 mg/ml) | | | | | | | |
| 8_{CAV} | CAV- NaCholate | NaCholate | 4.51 | 0.00 | 0.01 | 0.08 | 0.26 | 0.37 | 0.58 |
| | (2 × CMC)-citric | (13.46 mg/ml) | | | | | | | |
| | acid | Citric acid | | | | | | | |
| | | (4 mg/ml) | | | | | | | |
| 9_{CAV} | CAV- SLS | SLS | 9.27 | 0.02 | 0.04 | 0.08 | 0.17 | 0.24 | 0.36 |
| | (1:1 molar ratio) | (1.21 mg/ml) | | | | | | | |
| 10_{CAV} | CAV-DDM | DDM | 7.55 | 0.01 | 0.01 | 0.03 | 0.10 | 0.14 | 0.23 |
| | (1:1 molar ratio) | (2.07 mg/ml) | | | | | | | |
| 11_{CAV} | CAV-Tween21 | Tween 21 | 7.2 | 0.01 | 0.02 | 0.04 | 0.15 | 0.22 | 0.34 |
| | (1:1 molar ratio) | (2.41 mg/ml) | | | | | | | |
| 12_{CAV} | CAV- capric acid | Capric acid | 5.84 | 0.03 | 0.04 | 0.09 | 0.34 | 0.46 | 0.67 |
| | (1:2 molar ratio) | (1.5 mg/ml) | | | | | | | |
| 13_{CAV} | CAV- Citric acid | Citric acid | 5.84 | 0.08 | 0.12 | 0.23 | 0.67 | 0.89 | 1.19 |
| | (0.05mg/ml) | (0.05mg/ml) | | | | | | | |
| 14_{CAV} | CAV- Citric acid | Citric acid | 4.2 | 0.00 | 0.01 | 0.03 | 0.21 | 0.43 | 1.07 |
| | (0.5 mg/ml) | (0.5 mg/ml) | | | | | | | |
| 15_{CAV} | CAV- Aspartic | Aspartic acid | 4.8 | 0.16 | 0.29 | 0.57 | 2.45 | 3.22 | 4.73 |
| | acid (1:1 molar | (0.58 mg/ml) | | | | | | | |
| | ratio) | | | | | | | | |
| 16_{CAV} | CAV- Aspartic | Aspartic acid | 3.97^{a} | 0.09 | 0.22 | 0.50 | 1.81 | 2.84 | 4.87 |
| | acid (1:3 molar | (1.74 mg/ml) | | | | | | | |
| | ratio) | | | | | | | | |
| | (adjusted with | | | | | | | | |
| | 0.1M NaOH) | | | | | | | | |
| 17_{CAV} | CAV-Aspartic acid | Aspartic acid | 4.05^{a} | 0.03 | 0.13 | 0.34 | 1.60 | 2.42 | 4.36 |
| | (1:6 molar ratio) | (3.48 mg/ml) | | | | | | | |
| | (adjusted with | | | | | | | | |
| | 0.1M NaOH) | | | | | | | | |
| 18_{CAV} | CAV- Alanine | Alanine | 4.83^{b} | 0.15 | 0.22 | 0.41 | 1.03 | 1.53 | 2.31 |
| | (1:1 molar ratio) | (0.4 mg/ml) | | | | | | | |
| | (adjusted with | | | | | | | | |
| | 0.1M HCL) | | | | | | | | |
| 19_{CAV} | CAV-Span 80 | Span 80 | 7.77 | 0.01 | 0.01 | 0.01 | 0.04 | 0.05 | 0.12 |
| | (1:1 molar ratio) | (1.86 mg/ml) | | | | | | | |

^{a}pH after pH adjustment with 0.1M NaOH
^{b}pH after pH adjustment with 0.1M HCL

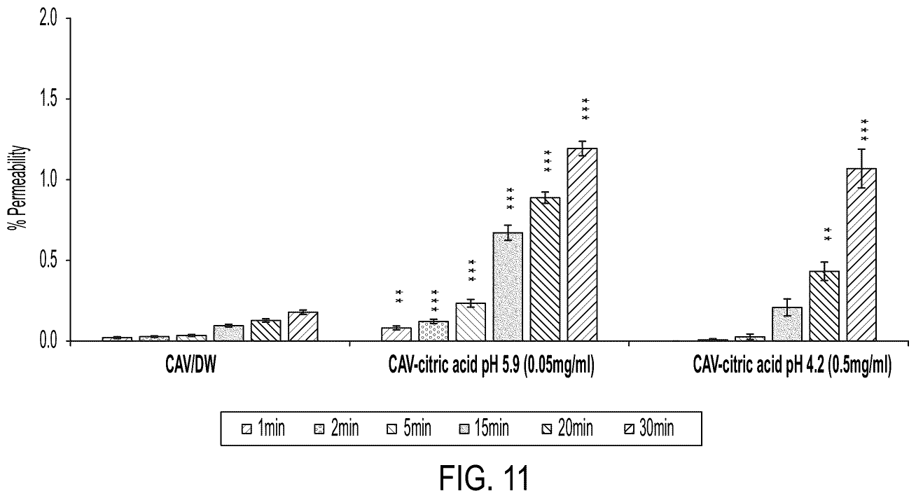

FIG. 11 depicts exemplary permeability data illustrating the effect of using a pH modifier as a permeability enhancer for Carvedilol. The effect of increasing the ionized fraction of Carvedilol on permeability was assessed by decreasing the pH of the Carvedilol solution with a weak acid such as citric acid. The pH was decreased from 7 (the Carvedilol control pH) to 5.9 and 4.2 with citric acid. Decreasing the pH increased the ionized fraction of Carvedilol from 86.32% (control) to 98.76% and 99.97%, respectively. Despite the low fraction of unionized drug (˜1%), the permeation assessment showed a significant increase (up to 7-fold) in percentage cumulative permeability compared to the Carvedilol control (which had ˜13.7% unionized drug). However, if the percentage of unionized drug is further reduced, the permeability enhancement may be impeded. Although drug molecules in the unionized state are favorable for permeation, the drug also needs to be in a solubilized state for absorption. The results suggest that because of the high lipophilicity of Carvedilol (log P of 3.05) in combination with the increase in drug solubility as pH decreases, a lower pH is sufficient to compensate for the low fraction of unionized drug and enable sufficient drug permeation. In contrast, at higher pH values, a lower proportion of the drug is solubilized, thus making less drug available for absorption. pH modification to improve the solubility of a highly lipophilic API may therefore be sufficient to increase permeability despite the low percentage of unionized drug.

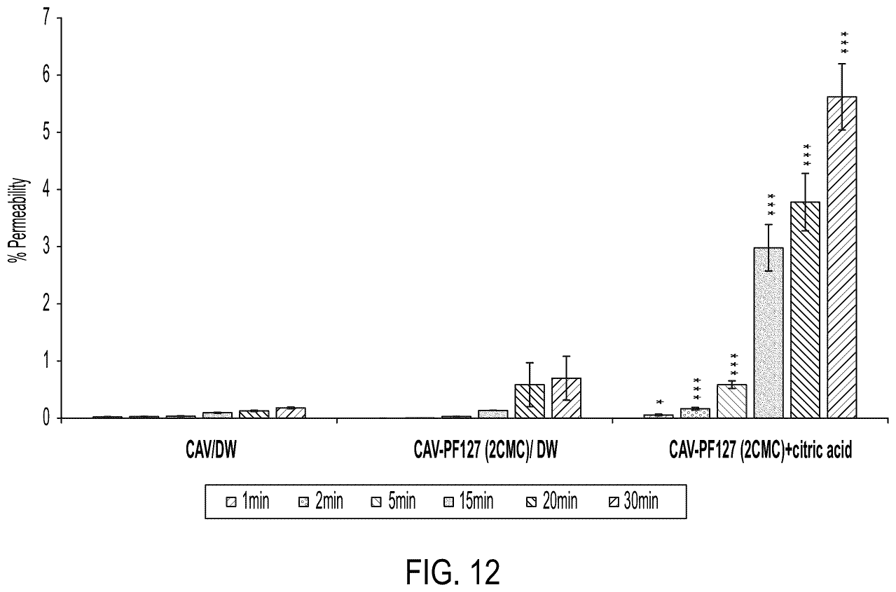

FIG. 12 depicts exemplary permeability data illustrating the effect of using a non-ionic surfactant, alone and in combination with a pH modifier, as a permeability enhancer for Carvedilol. Mixing PF127 at 2×CMC with Carvedilol showed no statistical difference in permeability. The lack of permeability enhancement at the initial phase may be due to the strong association of Carvedilol, a lipophilic substance, with the PF127 micelles. However, a sample prepared with both PF127 and citric acid showed a significant increase in permeability compared to the control sample. A 2-fold increase in percentage cumulative permeability was achieved after the first minute, and a 31-fold increase was achieved at 30 minutes. Citric acid enhances the permeability of Carvedilol by changing its ionization state and solubility. Thus, the presence of citric acid may reduce the association of Carvedilol with PF127, thereby increasing the influence of PF127 on the membrane and disturbing the lipid bilayer packing. This may explain the significant enhancement in Carvedilol permeability at lower pH values.

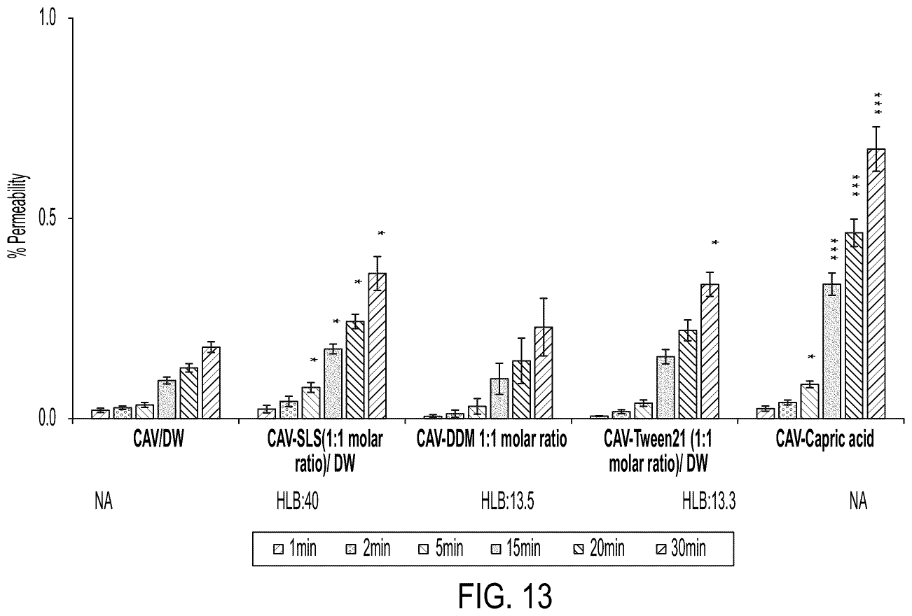

FIG. 13 depicts exemplary permeability data illustrating the effect of using various ionic and non-ionic surfactants and fatty acids as permeability enhancers for Carvedilol. All surfactants (SLS, Tween 21, DDM, and Span 80) were assessed at a 1:1 molar ratio to Carvedilol. SLS significantly increased permeability of Carvedilol, with a 2-fold increase achieved after 5 minutes and up to 30 minutes. Tween 21 significantly increased permeability only after 30 minutes. No significant effect on permeability was observed when DDM was used. Span 80 inhibited the permeability of Carvedilol.

FIG. 13 also shows the effect of fatty acids on the permeability of Carvedilol. Capric acid was used as a model medium-chain fatty acid (MCFA), with a C-10 carbon chain length. Carvedilol showed over a 3-fold increase in permeability after 30 minutes in the presence of capric acid. Fatty acids with chain lengths of C-10 or C-12 are hypothesized to display optimum activity when used for delivery through the buccal mucosa, which has a high content of polar lipids. This may explain the enhancement in Carvedilol permeability in the presence of capric acid.

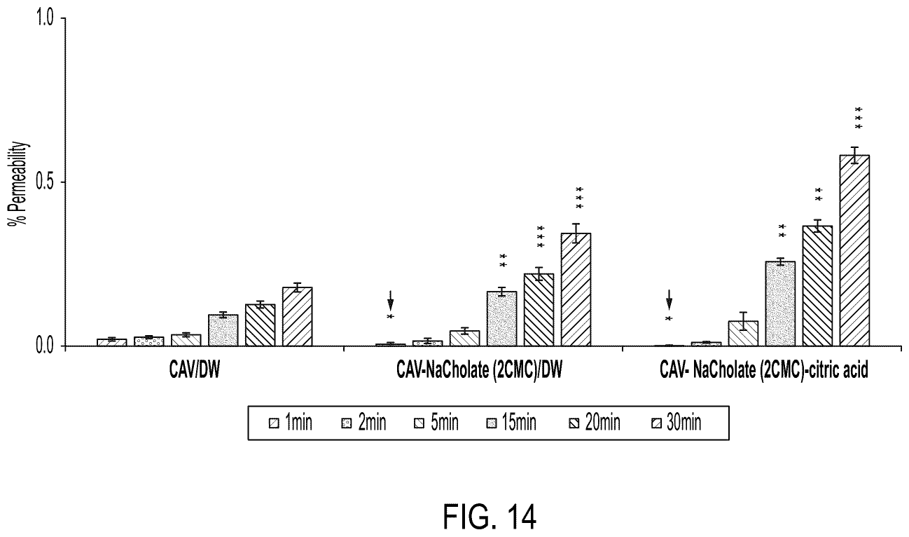

FIG. 14 depicts exemplary permeability data illustrating the effect of using bile salts and a combination of bile salts and pH modifiers as permeability enhancers for Carvedilol. Sodium cholate hydrate was used as a model bile salt. Sodium cholate hydrate significantly enhanced the permeability of Carvedilol after 15 minutes and displayed an almost 2-fold increase in the percentage cumulative permeability after 30 minutes. The sample containing both sodium cholate hydrate and citric acid displayed a similar trend as the sample containing PF127 and citric acid in combination, with a 3-fold increase in Carvedilol permeability compared to the control. The primary technique for bile salt to improve the permeability of Carvedilol is hypothesized to be increasing its water solubility through micellization and fluidizing the membrane, allowing the drug to passively permeate. The addition of citric acid enhances the solubility of Carvedilol, further increasing the permeability.

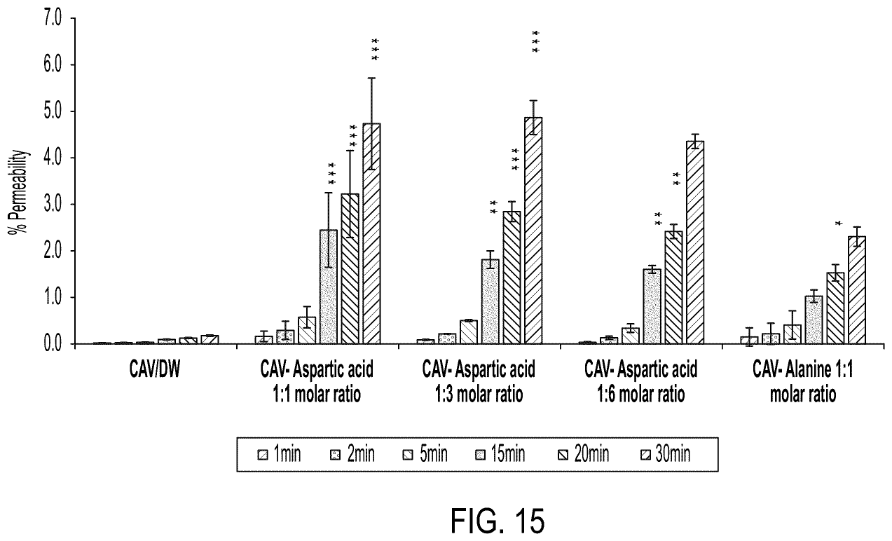

FIG. 15 depicts exemplary permeability data illustrating the effect of using various counterions as permeability enhancers for Carvedilol. Since Carvedilol has basic properties (pKa=7.8), aspartic acid, which is an acidic amino acid with pKa1=1.88, pKa2=3.65, and pKa3=9.60, was used as a model counterion. The effect of aspartic acid on Carvedilol permeability was assessed at 1:1, 1:3, and 1:6 molar ratios of Carvedilol to aspartic acid. Carvedilol permeability was significantly increased when aspartic acid was used at all molar ratios tested. The increase in percentage cumulative permeability ranged from 7 to 27-fold from the 1 minute to 30 minute time points of the experiment. Increasing the molar ratio of aspartic acid to Carvedilol did not further increase Carvedilol permeability. It is believed that amino acids as counterions may reduce the lipophilicity of a drug, and the drug's log P value may decrease as the amino acid ratio increases. Thus, increasing the ratio of counterions may not further increase the drug's permeability.

Alanine (pKa1=2.34, pKa2=9.69) was also studied as a model counterion at a 1:1 molar ratio to Carvedilol. The percentage cumulative permeability of Carvedilol increased 7 to 13-fold from 1 minute to 30 minutes in the presence of alanine.

### Example 3: Permeability Assessment of Famotidine

Famotidine (FAM) is a BCS Class III drug with a molecular weight of 337.5 g/mol. It is a weak base with a pKa of 6.76, a water solubility of 1 mg/ml, and a log P value of −0.64.

The permeability of Famotidine was assessed in combination with the following permeation agents: PF127, sodium cholate hydrate, Tween 21, DDM, aspartic acid, benzoic acid, and Glycine.

For this assessment, a control sample (1_{FAM}) containing Famotidine alone and twelve binary mixtures of Famotidine and permeation agents (2_{FAM}-13_{FAM}), each mixture containing 1.4 mg/ml of Famotidine, were prepared in distilled water. The concentrations of the permeation agents in the mixtures are summarized in Table 8. The samples were prepared at room temperature and stirred before they were sampled for testing. The pH of the mixtures were then measured, and the theoretical percent ionized and percent un-ionized of Famotidine at the respective pH values were calculated using the Henderson-Hasselbach equation.

TABLE 8
Summary of Famotidine/Permeation Agent binary mix samples

| | | Permeation | | | |
|---|---|---|---|---|---|
| Sample | | Agent & | | Calculated | Calculated |
| Reference | Sample name | concn (mg/ml) | pH | % ionised | % unionised |
| 1_{FAM} | Control | None | 8.57 | 1.53 | 98.47 |
| | (drug only) | | | | |
| 2_{FAM} | FAM-PF127 | PF127 | 8.21 | 3.43 | 96.57 |
| | (2 × CMC) | (1 mg/ml) | | | |
| 3_{FAM} | FAM-Tween 21 | Tween 21 | 8.44 | 2.05 | 97.95 |
| | (1:1 molar ratio) | (2.34 mg/ml) | | | |
| 4_{FAM} | FAM-DDM | DDM | 8.48 | 1.87 | 98.13 |
| | (1:1 molar ratio) | (2 mg/ml) | | | |
| 5_{FAM} | FAM-NaCholate | Sodium Cholate | 7.94 | 6.20 | 93.80 |
| | (2 × CMC) | (13.46 mg/ml) | | | |
| 6_{FAM} | FAM-Glycine | Glycine | 5.37^{a} | 96.09 | 3.91 |
| | (1:3 molar ratio); | (0.94 mg/ml) | | | |
| | pH adjusted with | | | | |
| | 0.1M HCL | | | | |
| 7_{FAM} | FAM-Glycine | Glycine | 8.11 | 4.28 | 95.72 |
| | (1:1 molar ratio) | (0.32 mg/ml) | | | |
| 8_{FAM} | FAM-Glycine | Glycine | 8.9^{b} | 0.72 | 99.28 |
| | (1:3 molar ratio); | (0.94 mg/ml) | | | |
| | pH adjusted with | | | | |
| | 0.1M NaOH | | | | |
| 9_{FAM} | FAM-Aspartic acid | Aspartic acid | 5.36 | 96.17 | 3.83 |
| | (1:1 molar ratio) | (0.56 mg/ml) | | | |
| 10_{FAM} | FAM-Aspartic acid | Aspartic acid | 5.29^{b} | 96.72 | 3.28 |
| | (1:3 molar ratio); | (1.65 mg/ml) | | | |
| | pH adjusted with | | | | |
| | 0.1M NaOH | | | | |
| 11_{FAM} | FAM-Aspartic acid | Aspartic acid | 8.77^{b} | 0.97 | 99.03 |
| | (1:1 molar ratio); | (0.56 mg/ml) | | | |
| | pH adjusted with | | | | |
| | 0.1M NaOH | | | | |
| 12_{FAM} | FAM-Benzoic acid | Benzoic acid | 5.35 | 96.26 | 3.74 |
| | (1:1 molar ratio) | (0.5 mg/ml) | | | |
| 13_{FAM} | FAM-Span 80 | Span 80 | 7.92 | 6.47 | 93.53 |
| | (1:1 molar ratio) | (1.82 mg/ml) | | | |

^{a}pH after adjustment with 0.1M HCL
^{b}pH after adjustment with 0.1M NaOH

For the permeability assessment. the control samples and samples of the binary mixtures summarized in Table 8 were tested by the procedure described in the Permeability Assessment Procedures section. Each sample was tested in triplicate. Prior to starting the permeability assessment, 1.5 ml of HBSS was added to each receiving chamber. After completion of the permeability assessment, 2.5 ml of methanol was added to each receiving chamber. The Transwell plates were then placed on a plate shaker at 150 rpm for two hours at room temperature. The samples were then prepared for HPLC analysis and assayed for Famotidine content.

To determine the drug concentration of Famotidine in the samples, an HPLC method was developed and validated. The method was optimized with chromatographic conditions of 55/45 v/v MeOH:H₂O (methanol; water with pH 3.3, adjusted with phosphoric acid), an Eclipse Plus HPLC column (3.5μ C18, 150×4.6 mm), 10 μl injection volume, a flow rate of 0.5 ml/min at ambient temperature (25° C.) and detected at 265 nm (UV spectrometry). The linearity range is 0.19-100 mcg/ml of Famotidine in methanol, acceptable linearity (R²=0.9998), and low LOD and LOQ (0.028 mcg/ml and 0.085 mcg/ml respectively). The run time was 6 minutes.

The results of the percentage cumulative permeability of Famotidine are summarized in Table 9.

TABLE 9
Permeability results of Famotidine/Permeation Agent
binary mixes - Percentage Cumulative Permeability

| | | Permeation | | % Cumulative Permeability (n = 3) | | | | | |
|---|---|---|---|---|---|---|---|---|---|
| Sample | | Agent & concn | | 1 | 2 | 5 | 15 | 20 | 30 |
| Ref | Sample name | (mg/ml) | pH | min | min | min | min | min | min |
| 1_{FAM} | Control | None | 8.57 | 0.32 | 0.84 | 2.09 | 7.81 | 11.76 | 19.98 |
| | (drug only) | | | | | | | | |
| 2_{FAM} | FAM-PF127 | PF127 | 8.21 | 0.43 | 0.96 | 4.00 | 7.18 | 10.23 | 16.29 |
| | (2 × CMC) | (1 mg/ml) | | | | | | | |
| 3_{FAM} | FAM-Tween 21 | Tween 21 | 8.44 | 0.37 | 0.81 | 1.69 | 7.96 | 11.84 | 18.32 |
| | (1:1 molar ratio) | (2.34 mg/ml) | | | | | | | |
| 4_{FAM} | FAM-DDM | DDM | 8.48 | 0.68 | 1.63 | 7.11 | 10.88 | 13.95 | 19.34 |
| | (1:1 molar ratio) | (2 mg/ml) | | | | | | | |
| 5_{FAM} | FAM-NaCholate | Sodium | 7.94 | 0.60 | 1.96 | 7.16 | 21.66 | 25.77 | 32.45 |
| | (2CMC) | Cholate | | | | | | | |
| | | (13.46 mg/ml) | | | | | | | |
| 6_{FAM} | FAM-Glycine | Glycine | 5.37^{a} | 0.74 | 1.64 | 3.84 | 9.23 | 12.38 | 17.64 |
| | (1:3 molar ratio); | (0.94 mg/ml) | | | | | | | |
| | pH adjusted with | | | | | | | | |
| | 0.1M HCL | | | | | | | | |
| 7_{FAM} | FAM-Glycine | Glycine | 8.11 | 0.62 | 1.66 | 5.69 | 19.77 | 24.32 | 29.34 |
| | (1:1 molar ratio) | (0.32 mg/ml) | | | | | | | |
| 8_{FAM} | FAM-Glycine | Glycine | 8.9^{b} | 0.61 | 1.67 | 5.65 | 20.93 | 25.57 | 32.13 |
| | (1:3 molar ratio); | (0.94 mg/ml) | | | | | | | |
| | pH adjusted with | | | | | | | | |
| | 0.1M NaOH | | | | | | | | |
| 9_{FAM} | FAM-Aspartic | Aspartic acid | 5.36 | 0.49 | 1.06 | 2.31 | 6.25 | 8.57 | 12.48 |
| | acid | (0.56 mg/ml) | | | | | | | |
| | (1:1 molar ratio) | | | | | | | | |
| 10_{FAM} | FAM-Aspartic | Aspartic acid | 5.29^{b} | 0.45 | 0.93 | 1.87 | 5.31 | 7.60 | 11.54 |
| | acid | (1.65 mg/ml) | | | | | | | |
| | (1:3 molar ratio); | | | | | | | | |
| | pH adjusted with | | | | | | | | |
| | 0.1M NaOH | | | | | | | | |
| 11_{FAM} | FAM-Aspartic | Aspartic acid | 8.77^{b} | 0.74 | 1.75 | 3.92 | 9.67 | 12.96 | 17.85 |
| | acid | (0.56 mg/ml) | | | | | | | |
| | (1:1 molar ratio); | | | | | | | | |
| | pH adjusted with | | | | | | | | |
| | 0.1M NaOH | | | | | | | | |
| 12_{FAM} | FAM-Benzoic | Benzoic acid | 5.35 | 0.50 | 1.05 | 2.28 | 6.10 | 8.60 | 12.72 |
| | acid | (0.5 mg/ml) | | | | | | | |
| | (1:1 molar ratio) | | | | | | | | |
| 13_{FAM} | FAM-Spam 80 | Span 80 | 7.92 | 1.05 | 1.79 | 4.42 | 14.01 | 20.29 | 33.74 |
| | (1:1 molar ratio) | (XX mg/ml) | | | | | | | |

^{a}pH after adjustment with 0.1M HCL
^{b}pH after adjustment with 0.1M NaOH

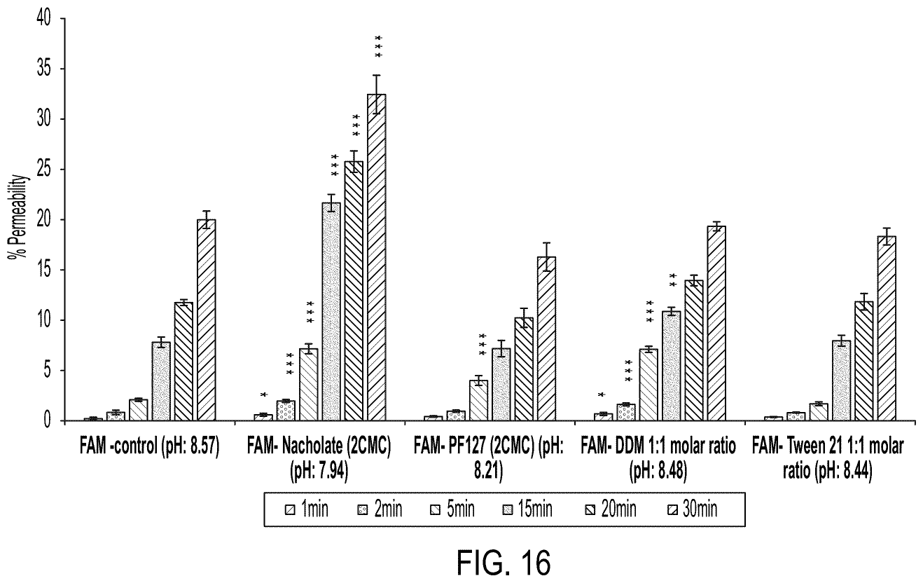

FIG. 16 depicts exemplary permeability data illustrating the effect of using bile salts and non-ionic surfactants as permeability enhancers for Famotidine. Model surfactants assessed included PF127, DDM, Tween 21, and Span 80. The model bile salt assessed was sodium cholate hydrate. All samples were prepared by mixing the surfactant or bile salt solutions with Famotidine solution and left to stir before carrying out the permeability assessment. Neither PF127 nor Tween 21 showed an enhancement in Famotidine permeability. DDM in a 1:1 molar ratio with Famotidine showed a significant increase in Famotidine permeability. Permeability increased 3-fold compared to the control sample in the first 5 through 15 minutes, though no significant difference in permeability was observed at the 20 and 30 minute marks. Span 80 in a 1:1 molar ratio with Famotidine showed a 2-fold increase in permeability compared to the control sample. Sodium cholate hydrate at 2×CMC showed a significant increase in Famotidine permeability, with a 2 to 3.5-fold increase in percentage cumulative permeability compared to the Famotidine control at all time points in the experiment.

For sodium cholate hydrate and DDM, the findings were dissimilar to those achieved when tested with Atenolol, which is another BCS Class III drug. However, Famotidine and Atenolol have very different solubility and lipophilicity characteristics. Famotidine is less hydrophilic (water solubility of 1 mg/ml) and less permeable (log P value of −0.64) compared to Atenolol, which has a water solubility of 13.5 mg/ml and log P value of 0.57. Permeation agents like bile salts may enhance permeability of a drug by disrupting the cell membrane. Thus, when sodium cholate hydrate and DDM were used, the water solubility difference between Famotidine and Atenolol may have contributed to the difference in permeability.

For PF127 and Tween 21, no increase in Famotidine permeability was observed. When PF127 and Tween 21 are above their CMC, they spontaneously form micelles with a hydrophilic exterior and a hydrophobic interior. PF127 and Tween 21 may not have improved permeability because the hydrophilic Famotidine molecule was not enclosed within the hydrophobic micelle core. This could also explain the similar results achieved when PF127 and Tween 21 were used with Atenolol.

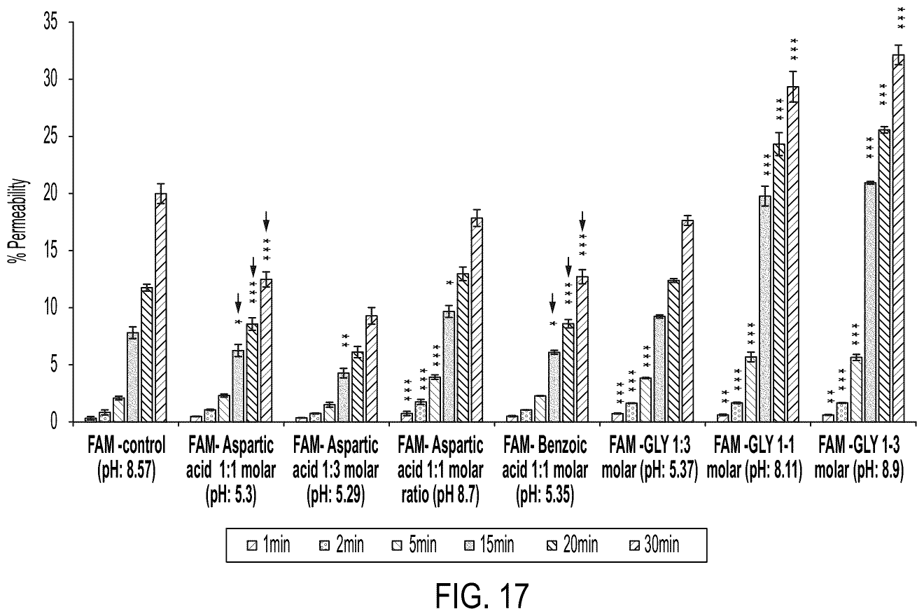

FIG. 17 depicts exemplary permeability data illustrating the effect of using various counterions as permeability enhancers for Famotidine. Famotidine has basic properties, with a pKa of 6.76 and four basic NH₂ functional groups. Aspartic acid (pKa1=1.88, pKa2=3.65, pKa3=9.60), benzoic acid (pKa 4.20), and glycine (pKa 1=2.34, pKa2=9.60) were selected as counterions. Samples of aspartic acid at 1:1 and 1:3 molar ratios to Famotidine (Samples 9_{FAM} and 10_{FAM}), benzoic acid at a 1:1 molar ratio to Famotidine (Sample 12_{FAM}), and glycine at a 1:3 molar ratio to Famotidine (Sample 6_{FAM}) were prepared at a pH two units different from the pKa of both the Famotidine and the counterion to ensure that both were fully ionized. As a result, the samples' pH values ranged from 5.0-5.5. All four samples failed to show any increase in Famotidine permeability. At a pH of 5, all four functional groups of Famotidine may have been ionized, and in the presence of ionized counterions, ion pairs could be formed on all four groups, which could increase the bulk structure of the drug-ion pair complex and decrease its permeability. Thus, if the Famotidine was only partially ionized, it may be able to form smaller drug-counterion ion pair complexes, which may increase permeability. To test this hypothesis, samples of Famotidine with aspartic acid at a 1:1 molar ratio (Sample 11_{FAM}) and glycine at 1:1 (Sample 7_{FAM}) and 1:3 (Sample 8_{FAM}) molar ratios were prepared with pH values of 8-9. All three samples showed a significant (2 to 3-fold) increase in Famotidine permeability compared to the control at all time points. At this pH range, a smaller number of ion pairs were formed, and the reduced hindrance from ionized functional groups increased the permeability of Famotidine.

### Example 4: Permeability Assessment of Atenolol

Atenolol (ATL) is a BCS Class III drug with a molecular weight of 266.34 g/mol. It is a weak base with a pKa of 9.6, a water solubility of 13.5 mg/ml, and a log P value of 0.57.

The permeability of Atenolol was assessed in combination with the following permeation agents: PF127, SLS, Tween 21, DDM, Span 80, sodium cholate hydrate, citric acid, aspartic acid, and benzoic acid.

For this assessment, a control sample (1_{ATL}) containing Atenolol alone and thirteen binary mixtures of Atenolol and permeation agents (2_{ATL}-14_{ATL}), each mixture containing 1.74 mg/ml of Atenolol, were prepared in distilled water. The concentrations of the permeation agents in the mixtures are summarized in Table 10. The samples were prepared at room temperature and stirred before they were sampled for testing. The pH of the mixtures were then measured, and the theoretical percent ionized and percent un-ionized of Atenolol at the respective pH values were calculated using the Henderson-Hasselbach equation.

TABLE 10
Summary of Atenolol: Permeation Agent binary mix samples

| Sample | | Permeation agent & | | % | % |
|---|---|---|---|---|---|
| Reference | Sample name | concn (mg/ml) | pH | ionised | unionised |
| 1_{ATL} | Control | None | 10.58 | 9.48 | 90.52 |
| | (drug only) | | | | |
| 2_{ATL} | ATL -PF127 | PF127 | 10.6 | 9.09 | 90.91 |
| | (2 × CMC) | (1 mg/ml) | | | |
| 3_{ATL} | ATL-SLS | SLS | 10.9 | 4.77 | 95.23 |
| | (1:1 molar ratio) | (1.88 mg/ml) | | | |
| 4_{ATL} | ATL-Tween21 | Tween 21 | 10.46 | 12.13 | 87.87 |
| | (1:1 molar ratio) | (3.68 mg/ml) | | | |
| 5_{ATL} | ATL- DDM | DDM | 10.66 | 8.01 | 91.99 |
| | (1:1 molar ratio) | (3.15 mg/ml) | | | |
| 6_{ATL} | ATL-Span 80 | Span 80 | 9.15 | 73.81 | 26.19 |
| | (1:1 molar ratio) | (2.8 mg/ml) | | | |
| 7_{ATL} | ATL-NaCholate | NaCholate | 10.47 | 11.89 | 88.11 |
| | (2 × CMC) | (13.46 mg/ml) | | | |
| 8_{ATL} | ATL- citric acid | Citric acid | 8.84 | 85.19 | 14.81 |
| | (0.37 mg/ml) | (0.37 mg/ml) | | | |
| 9_{ATL} | ATL- citric acid | Citric acid | 6.66 | 99.89 | 0.11 |
| | (0.5 mg/ml) | (0.5 mg/ml) | | | |
| 10_{ATL} | ATL-citric acid | Citric acid | 4.45 | 100.00 | 0.00 |
| | (1 mg/ml) | (1 mg/ml) | | | |
| 11_{ATL} | ATL- Aspartic acid | Aspartic acid | 10.56 | 9.88 | 90.12 |
| | (3:1 molar ratio) | (0.58 mg/ml) | | | |
| 12_{ATL} | ATL- Aspartic acid | Aspartic acid | 5.7 | 99.99 | 0.01 |
| | (1:1 molar ratio) | (0.87 mg/ml) | | | |
| 13_{ATL} | ATL-Aspartic acid | Aspartic acid | 6.3^{a} | 99.95 | 0.05 |
| | (1:3 molar ratio); | (2.61 mg/ml) | | | |
| | pH adjusted with | | | | |
| | 0.1M NaOH | | | | |
| 14_{ATL} | ATL- benzoic acid | Benzoic acid | 7.5^{a} | 99.21 | 0.79 |
| | (1:1 molar ratio); | (0.78 mg/ml) | | | |
| | pH adjusted with | | | | |
| | 0.1M NaOH | | | | |

^{a}pH after pH adjustment with 0.1M NaOH

For the permeability assessment, the control samples and samples of the binary mixtures summarized in Table 10 were tested by the procedure described in the Permeability Assessment Procedures section. For each test sample (done in triplicate), 0.5 ml of the sample was added to the donor chamber of the Transwell plate. Prior to starting the permeability assessment, 1.5 ml of HBSS was added to each receiving chamber. After completion of the permeability assessment, 1.5 ml of acetonitrile was added to each receiving chamber. The Transwell plates were then placed on a plate shaker at 150 rpm for two hours at room temperature. The samples were then prepared for HPLC analysis and assayed for Atenolol content.

To determine the drug concentration of Atenolol in the samples, an HPLC method was developed and validated. The method was optimized with chromatographic conditions of 60/40 TFA/ACN (trifluoroacetic acid; acetonitrile), 0.1% TFA, a Phenomenex HPLC column (Gemini 5μ C18, 150×4.6 mm), 20 μl injection volume, a flow rate of 0.5 ml/min at ambient temperature (25° C.) and detected at excitation 276 nm and emission 296 nm fluorescence. The linearity range is 0.19-100 μg/ml of Atenolol in ACN, acceptable linearity (R²=1), and low LOD and LOQ (0.02 μg/ml and 0.05 μg/ml respectively).

The results of the percentage cumulative permeability of Atenolol are summarized in Table 11.

TABLE 11
Permeability results of Atenolol/Permeation Agent binary mixes

| | | Permeation | | % Cumulative Permeability (n = 3) | | | | | |
|---|---|---|---|---|---|---|---|---|---|
| Sample | | agent & concn | | 1 | 2 | 5 | 15 | 20 | 30 |
| Ref | Sample name | (mg/ml) | pH | min | min | min | min | min | min |
| 1_{ATL} | Control | None | 10.58 | 0.05 | 0.12 | 0.35 | 1.03 | 1.31 | 1.77 |
| | (drug only) | | | | | | | | |
| 2_{ATL} | ATL -PF127 | PF127 | 10.6 | 0.04 | 0.10 | 0.29 | 0.88 | 1.14 | 1.67 |
| | (2 × CMC) | (1 mg/ml) | | | | | | | |
| 3_{ATL} | ATL-SLS | SLS | 10.9 | 0.25 | 0.37 | 0.60 | 1.11 | 1.38 | 1.76 |
| | (1:1 molar ratio) | (1.88 mg/ml) | | | | | | | |
| 4_{ATL} | ATL-Tween21 | Tween 21 | 10.46 | 0.05 | 0.13 | 0.34 | 0.89 | 1.17 | 1.60 |
| | (1:1 molar ratio) | (3.68 mg/ml) | | | | | | | |
| 5_{ATL} | ATL- DDM | DDM | 10.66 | 0.03 | 0.12 | 0.35 | 0.93 | 1.22 | 1.69 |
| | (1:1 molar ratio) | (3.15 mg/ml) | | | | | | | |
| 6_{ATL} | ATL-Span 80 | Span 80 | 9.15 | 0.04 | 0.10 | 0.37 | 1.03 | 1.32 | 1.80 |
| | (1:1 molar ratio) | (2.8 mg/ml) | | | | | | | |
| 7_{ATL} | ATL-NaCholate | NaCholate | 10.47 | 0.06 | 0.18 | 0.38 | 1.05 | 1.26 | 1.74 |
| | (2CMC) | (13.46 mg/ml) | | | | | | | |
| 8_{ATL} | ATL- citric acid | Citric acid | 8.84 | 0.02 | 0.05 | 0.12 | 0.50 | 0.73 | 1.14 |
| | (0.37 mg/ml) | (0.37 mg/ml) | | | | | | | |
| 9_{ATL} | ATL- citric acid | Citric acid | 6.66 | 0.03 | 0.06 | 0.17 | 0.58 | 0.82 | 1.23 |
| | (0.5 mg/ml) | (0.5 mg/ml) | | | | | | | |
| 10_{ATL} | ATL-citric acid | Citric acid | 4.45 | 0.02 | 0.04 | 0.10 | 0.34 | 0.47 | 0.69 |
| | (1 mg/ml) | (1 mg/ml) | | | | | | | |
| 11_{ATL} | ATL- Aspartic acid | Aspartic acid | 10.56 | 0.03 | 0.11 | 0.28 | 0.89 | 1.17 | 1.89 |
| | (3:1 molar ratio) | (0.58 mg/ml) | | | | | | | |
| 12_{ATL} | ATL- Aspartic acid | Aspartic acid | 5.7 | 0.39 | 0.92 | 2.25 | 6.47 | 9.07 | 12.07 |
| | (1:1 molar ratio) | (0.87 mg/ml) | | | | | | | |
| 13_{ATL} | ATL-Aspartic acid | Aspartic acid | 6.3^{a} | 0.15 | 0.40 | 1.33 | 3.92 | 5.13 | 8.18 |
| | (1:3 molar ratio); | (2.61 mg/ml) | | | | | | | |
| | pH adjusted with | | | | | | | | |
| | 0.1M NaOH | | | | | | | | |
| 14_{ATL} | ATL- benzoic acid | Benzoic acid | 7.5^{a} | 0.22 | 0.50 | 1.46 | 4.78 | 6.45 | 9.55 |
| | (1:1 molar ratio); | (0.78 mg/ml) | | | | | | | |
| | pH adjusted with | | | | | | | | |
| | 0.1M NaOH | | | | | | | | |

^{a}pH after pH adjustment with 0.1M NaOH

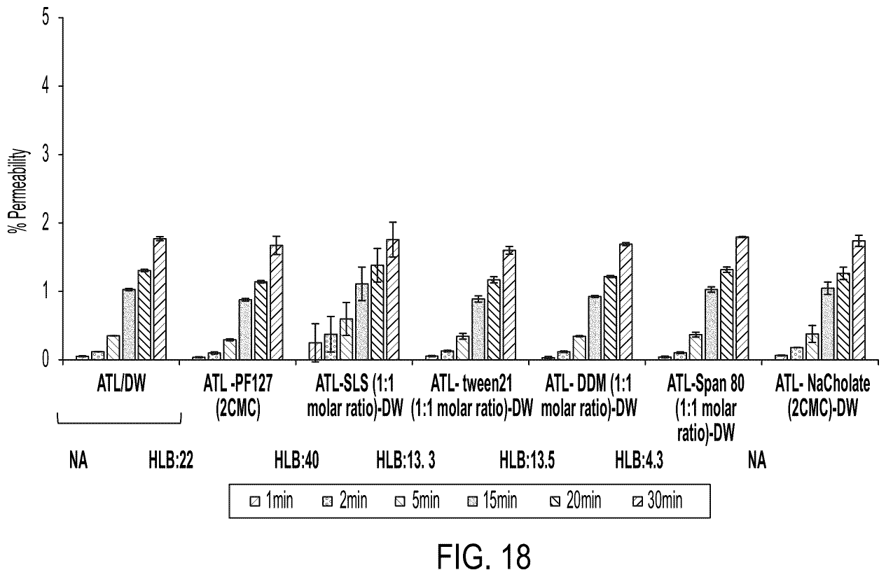

FIG. 18 depicts exemplary permeability data illustrating the effect of using various ionic and non-ionic surfactants and bile salts as permeability enhancers for Atenolol. Span 80, Tween 21, SLS, and DDM were assessed at a 1:1 molar ratio to Atenolol. PF127 was assessed at a concentration of 2 CMC. Surfactants may improve drug permeability by disturbing the lipid membrane and solubilizing the drug, followed by micellization. However, in this experiment, neither lipophilic (HLB<10) nor hydrophilic (HLB>10) surfactants showed any significant increase in Atenolol permeability. SLS showed a non-statistically significant increase in permeability compared to the control for the first 5 minutes and showed no difference thereafter. PF127, DDM, Tween 21, and Span 80 all showed no statistically significant difference in drug permeability when compared to the control. Although surfactants may potentially impact the lipid bilayer packing of the membrane, a drug with hydrophilic functional groups like Atenolol may still be unable to pass through the lipophilic membrane. The hydrophilic nature of Atenolol also renders it unlikely to be enclosed within the core of micelles.

FIG. 18 also shows the effect of bile salts on the permeability of Atenolol compared to the Atenolol control sample. Sodium cholate hydrate was used as a model bile salt. Its effect on Atenolol permeability was assessed at a concentration of 2×CMC. Bile salts may improve drug permeability by increasing the water solubility through micellization and fluidizing the membrane, thus allowing the drug to undergo passive permeation. However, this experiment showed no statistical difference in permeability of the bile salt-Atenolol mixture when compared to the Atenolol control. Despite membrane fluidization, sodium cholate hydrate had no effect on Atenolol permeability because of the inability of the hydrophilic drug to cross the lipid-rich cell membrane.

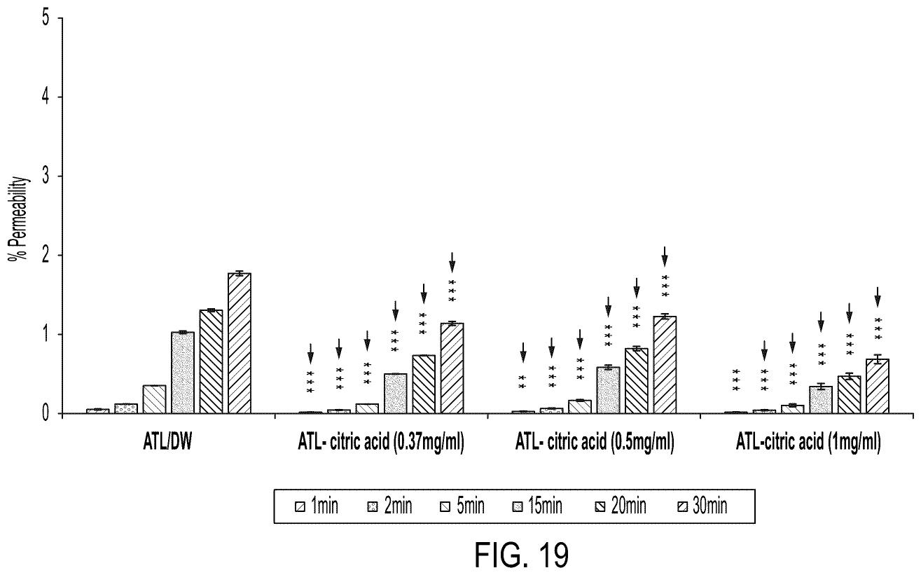

FIG. 19 depicts exemplary permeability data illustrating the effect of using PH modifiers as permeability enhancers for Atenolol. Citric acid was used as a model pH modifier for Atenolol, which is a weak base. The permeability of a drug can be influenced by the relative fractions of ionized drug to unionized drug, which may be manipulated by modifying the pH of the API dispersion with a pH modifier such as a weak acid, weak base, or buffer. Unionized drug is required for absorption. Citric acid was evaluated at concentrations of 0.37 mg/ml, 0.5 mg/ml, and 1 mg/ml, giving rise to pH values of 8.84, 6.66, and 4.45, respectively. The corresponding calculated percentages of unionized drug at each pH were 14.81%, 0.11%, and 0%, respectively. In comparison, the Atenolol control sample had a pH of 10.58, and the calculated percentage of unionized drug was 90.52%. A statistically significant reduction in percentage cumulative permeability compared to the Atenolol control sample was observed for the samples containing citric acid. For example, at pH 6.6 with 0.5 mg/ml citric acid, when Atenolol was 0.11% unionized, the percentage cumulative permeability at 30 minutes was reduced from 1.77% (control) to 1.23%. The permeability reduction was comparable for the samples containing 0.37 mg/ml and 0.5 mg/ml citric acid. The reduction was slightly more pronounced for the 1 mg/ml citric acid sample. Since Atenolol is a weak base and a BCS Class III API, it is permeability limiting. Thus, when the pH was decreased, the reduction in the percentage of unionized drug, rather than the increase in solubility, had a bigger impact on the permeability of the API. pH modification may be used to either improve or inhibit permeation of a drug depending on whether the drug is a weak acid or base and its pKa and solubility characteristics.

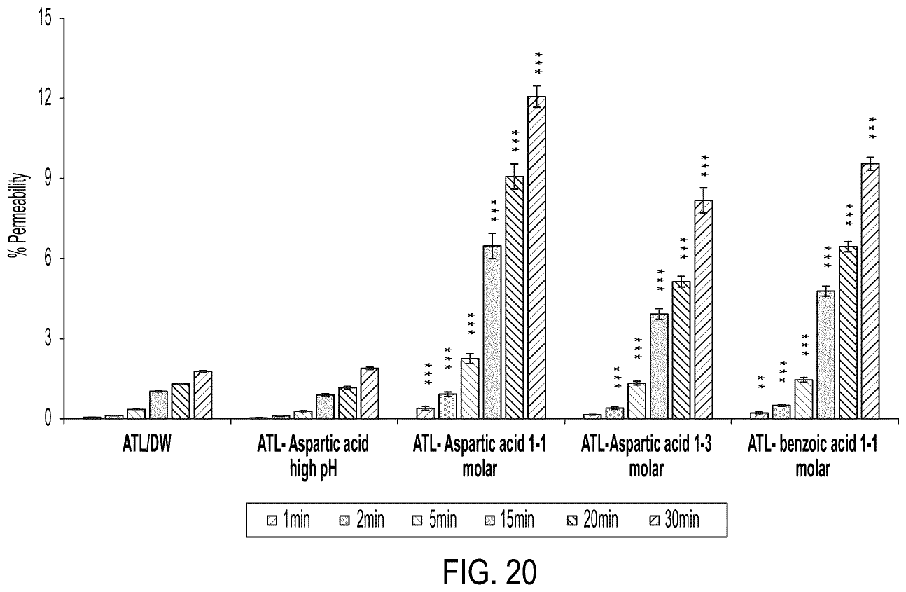

FIG. 20 depicts exemplary permeability data illustrating the effect of using various counterions as permeability enhancers for Atenolol. Since Atenolol is a weak base (pKa=9.6), benzoic acid (pKa=4.20) and aspartic acid (pKa1=1.88, pKa2=3.65 and pKa3=9.60) were selected as counterions. All samples were prepared at a pH 2 units different from the pKa of both the drug and the counterions, where both were fully ionized. The pH values of the samples were adjusted to 5.5-6.5 for aspartic acid and 7 for benzoic acid. Aspartic acid was studied at molar ratios of 3:1, 1:1, and 1:3 aspartic acid to Atenolol. The 3:1 molar ratio, which resulted in a pH of 10.56, was not pH-adjusted. This pH was similar to the Atenolol control sample, which had a pH of 10.58. The permeability of the 3:1 molar ratio sample was comparable to the Atenolol control sample, which confirmed the need to adjust the pH of the sample to ensure ionization of both the drug and counterion to obtain an ion pair. The 1:1 molar ratio sample had a pH of 5.7, which was within the accepted range and did not require a pH adjustment. The 1:1 sample showed a 7-fold increase in Atenolol permeability compared to the Atenolol control sample. The 1:3 molar ratio sample, which had a pH of 3.3, required a pH adjustment to 6.3 using 0.1M NaOH. The 1:3 sample showed a 4-fold increase in Atenolol permeability compared to the control sample. The lower permeability compared to the 1:1 sample could be due to the amino acid reducing the lipophilicity of the drug when present at a higher concentration. Additionally, an increase in the size of the ion pair complex may also reduce permeability. Benzoic acid was assessed at a 1:1 molar ratio to Atenolol and required a pH adjustment with 0.1M NaOH from pH 5.4 to 7.5. Benzoic acid samples showed a 4 to 4.5-fold increase in permeability compared to the Atenolol control sample.

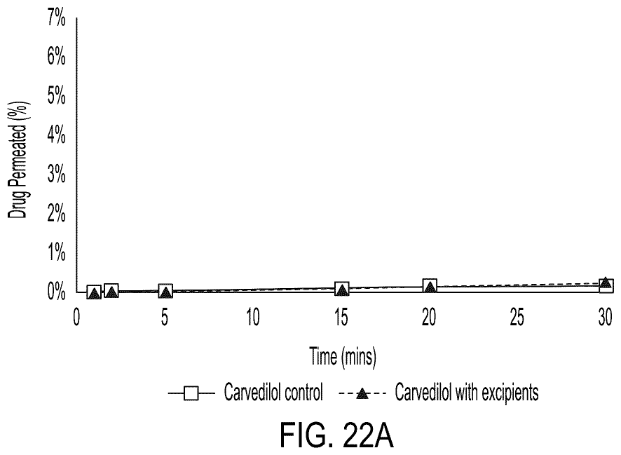

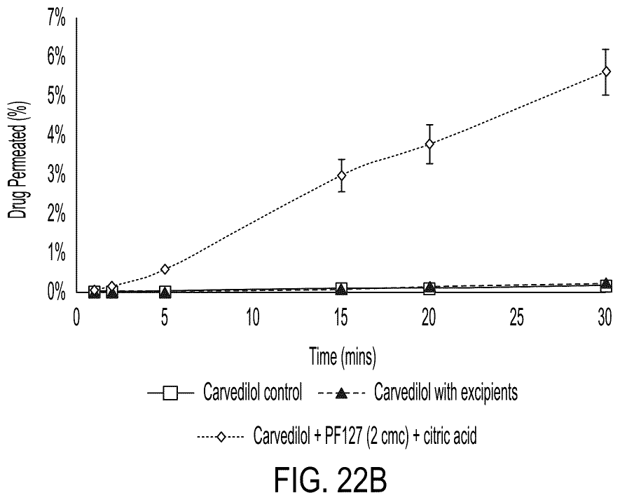

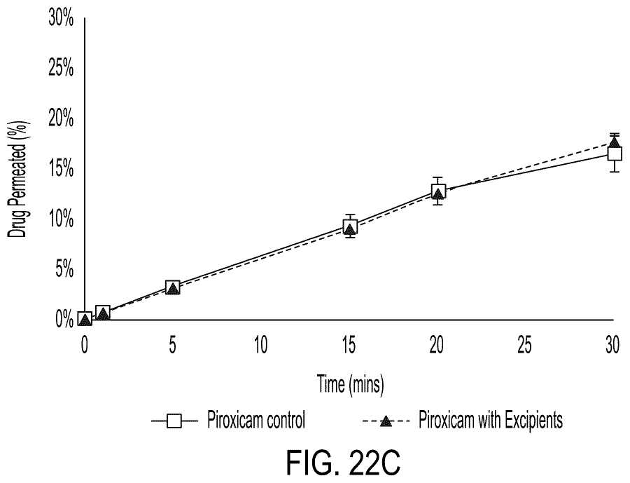

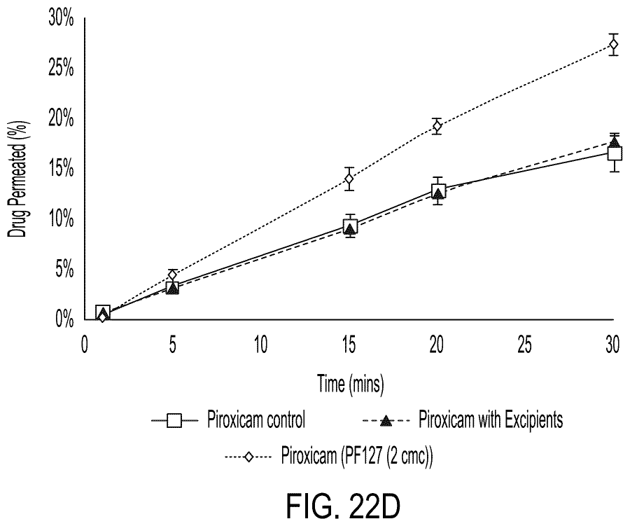

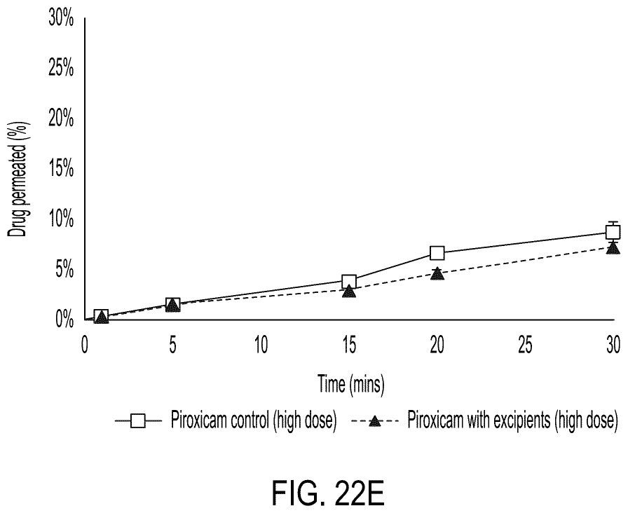

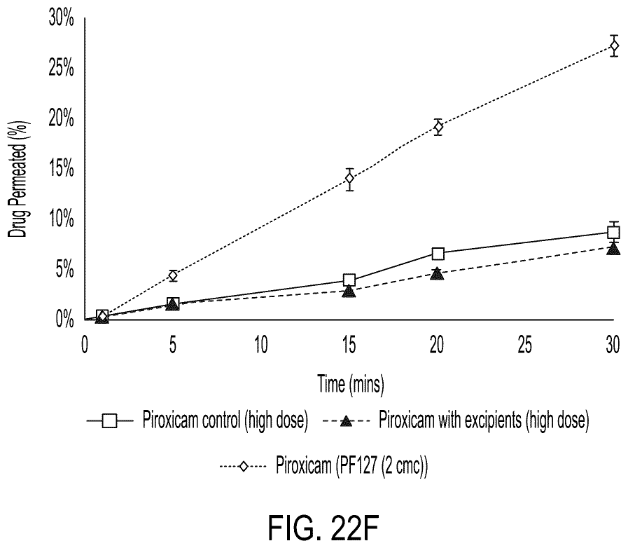

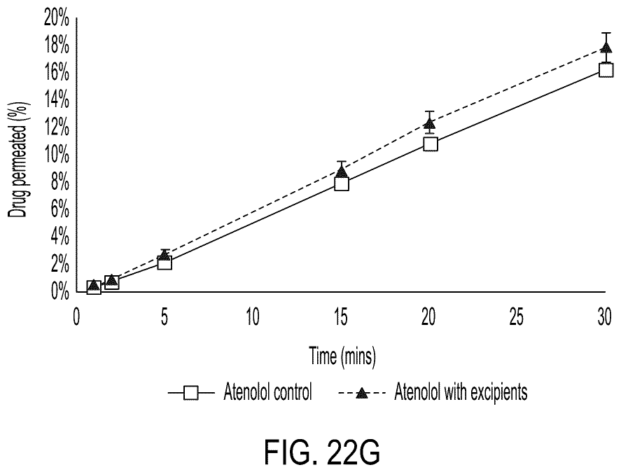

The permeability enhancement shown in binary mixture formulations can be directly correlated to permeability enhancement in a pharmaceutical composition (freeze-dried orally dissolvable tablet). This is because the base matrix has been shown to be inert to permeability as shown in FIGS. 22A-G. In these examples, the base matrix added to the pharmaceutical formulations included 5 wt. % gelatin and 4 wt. % mannitol for samples with excipients. The control samples simply added water. These samples also included a drug (piroxcam at 0.35 mg (low dose) and 2 mg (high dose); atenolol at 0.87 mg; carvedilol at 0.87 mg) in 0.5 mL.

FIG. 22A illustrates exemplary permeability data illustrating that the base matrix excipients have no effect on the permeability of carvedilol. FIG. 22B illustrates exemplary permeability data illustrating that the base matrix excipients have no effect on permeability of carvedilol versus a positive effect with a non-ionic surfactant (PF127). FIG. 22C illustrate exemplary permeability data illustrating that base matrix excipients have no effect on permeability of piroxicam (at low dose). FIG. 22D illustrates exemplary permeability data illustrating that base matrix (with excipients) has no effect on permeability of piroxicam (at low dose) versus a positive effect of a non-ionic surfactant (PF127). FIG. 22E illustrates exemplary permeability data illustrating that base matrix excipients have no effect on permeability of piroxicam (at high dose). FIG. 22F illustrates exemplary permeability data illustrating that base matrix excipients has no effect on permeability of piroxicam (at low dose) versus a positive effect of a non-ionic surfactant (PF127). FIG. 22G illustrates exemplary permeability data illustrating that base matrix excipients have no enhancement effect on the permeability of atenolol.

In other words, the additional materials added to form the pharmaceutical formulation (e.g., structure former (mannitol), matrix former (gelatin)) do not have an effect on the permeability. In addition, as shown by the data herein, any enhancement, inhibition, or no change in permeability in a tested binary formulation (API & permeation enhancer) was also shown when the corresponding dosage form was tested.

FIG. 3 depicts an example data set determined by the Applicant which can indicate combinations of API BCS information and sub-classification information and suitable PEs associated respectively with each of the combinations of API BCS information and sub-classification information. The data set illustrates the performance of various PEs, including hydrophilic surfactants, lipophilic surfactants, bile salts, micelles, fatty acids, counterions, and pH modifiers, when used with APIs having various BCS classifications and sub-classifications. Column 1 indicates the performance of the aforementioned PEs for a practically insoluble BCS Class II API. Column 2 indicates the performance of the aforementioned PEs for a very slightly soluble BCS Class II API. Column 3 indicates the performance of the aforementioned PEs for a slightly soluble BCS Class III API. Column 4 indicates the performance of the aforementioned PEs for a sparingly soluble BCS Class III API.

FIG. 21 is a decision-making flowchart that illustrates how the data set in FIG. 3 can be used to identify PEs suitable for use with an API associated with a given combination of API BCS information and sub-classification information. Appropriate PEs to combine with the API can be determined by the flowchart from left to right. For example, an API may be identified as BCS Class II. Class II APIs may be further sub-classified as “practically insoluble,” with solubility less than 0.1 mg/ml and/or a log P value greater than 2.5, or “very slightly soluble,” with solubility between 0.1 and 1 mg/ml and/or a log P value between 1 and 2.5. If the API's sub-classification is “practically insoluble,” the flowchart indicates that suitable PEs for enhancing permeability include counterions, pH modifiers, surfactants with HLB values greater than 10, bile salts, micelles, and fatty acids (and also the potential to adjust the pH to improve solubility). Alternatively, if the API's sub-classification is “very slightly soluble,” suitable PEs for enhancing permeability include counterions, pH modifiers, surfactants with HLB values greater than 10, bile salts, and micelles (and also the potential to adjust the pH to improve solubility). For very slightly soluble APIs, the flowchart also indicates PEs that may inhibit permeation, such as surfactants with HLB values less than 10 and fatty acids. In another example, the API may be classified as BCS Class III. Class III APIs may further be sub-classified as “slightly soluble,” with solubility between 1 and 10 mg/ml and/or a log P value less than 1, or “sparingly soluble,” with solubility between 10 and 33 mg/ml and/or a log P value less than 1. If the API's sub-classification is “slightly soluble,” suitable PEs for enhancing permeability include counterions, bile salts, and pH modifiers (and also the potential to adjust the pH to improve solubility). The flowchart also indicates PEs that may only slightly enhance or have no effect on permeability, including micelles and surfactants with HLB values greater than 10. Alternatively, if the API's sub-classification is “sparingly soluble,” suitable PEs for enhancing permeability include counterions and pH modifiers (and also the potential to adjust the pH to improve solubility). The flowchart also indicates PEs that may inhibit permeability, including surfactants (with both HLB values greater than 10 and less than 10), bile salts, and micelles.

### Additional Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Reference to “about” a value or parameter herein includes (and describes) variations that are directed to that value or parameter per se. For example, description referring to “about X” includes description of “X”. In addition, reference to phrases “less than”, “greater than”, “at most”, “at least”, “less than or equal to”, “greater than or equal to”, or other similar phrases followed by a string of values or parameters is meant to apply the phrase to each value or parameter in the string of values or parameters. For example, a statement that a solution has a concentration of at least about 10 mM, about 15 mM, or about 20 mM is meant to mean that the solution has a concentration of at least about 10 mM, at least about 15 mM, or at least about 20 mM.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

The above description is presented to enable a person skilled in the art to make and use the disclosure, and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

1. A pharmaceutical composition comprising:
a pharmaceutically effective amount of a biopharmaceutics classification system (BCS) Class II active pharmaceutical ingredient (API) or pharmaceutically acceptable salt or solvate thereof having a solubility of less than 0.1 mg/mL and a log P value greater than 2.5;
a permeation enhancer comprising at least one selected from the group of counterions, a pH modifier, a surfactant with a hydrophilic lipophilic balance (HLB) greater than 10, a bile salt, a micelle, or a fatty acid;
a matrix former; and
a structure former.

2. The pharmaceutical composition of claim 1, wherein the permeation enhancer comprises a surfactant with a hydrophilic lipophilic balance (HLB) greater than 10.

3. The pharmaceutical composition of claim 2, wherein a molar ratio of API to the surfactant is 5:1 to 1:5 and/or the pharmaceutical composition comprises 0.5-20 wt. % the surfactant.

4. The pharmaceutical composition of claim 2, further comprising a pH modifier.

5. The pharmaceutical composition of claim 1, wherein the permeation enhancer comprises a bile salt.

6. The pharmaceutical composition of claim 5, wherein a molar ratio of API to the bile salt is 15:1 to 1:15 and/or the pharmaceutical composition comprises 1-20 wt. % the bile salt.

7. The pharmaceutical composition of claim 1, wherein the permeation enhancer comprises a fatty acid.

8. The pharmaceutical composition of claim 7, wherein a molar ratio of API to fatty acid is 3:1 to 1:3 and/or the pharmaceutical composition comprises 0.25-5 wt. % fatty acid.

9. The pharmaceutical composition of claim 1, wherein the permeation enhancer comprises counterions.

10. The pharmaceutical composition of claim 1, wherein a molar ratio of API to counterions is 6:1 to 1:6 and/or the pharmaceutical composition comprises 0.1-25 wt. % counterions.

11. The pharmaceutical composition of claim 9, further comprising a pH modifier.

12. The pharmaceutical composition of claim 1, further comprising 25-60 wt. % matrix former.

13. The pharmaceutical composition of claim 12, wherein the matrix former comprises gelatin, pullulan, starch, or combinations thereof.

14. The pharmaceutical composition of claim 3, wherein the gelatin comprises fish gelatin, bovine gelatin, porcine gelatin, or combination thereof.

15. The pharmaceutical composition of claim 4, wherein the gelatin is fish gelatin and the fish gelatin is high molecular weight fish gelatin.

16. The pharmaceutical composition of claim 1, further comprising 20-45 wt. % structure former.

17. The pharmaceutical composition of claim 16, wherein the structure former comprises mannitol.

18. The pharmaceutical composition of claim 1, wherein the pharmaceutical composition comprises 1-35 wt. % the BCS Class II API or pharmaceutically acceptable salt or solvate thereof.

19-59. (canceled)

60. A method of forming a solid dosage form, the method comprises:
dosing a pharmaceutical formulation into a preformed mold, wherein the pharmaceutical formulation comprises:
a pharmaceutically effective amount of a biopharmaceutics classification system (BCS) Class II active pharmaceutical ingredient (API) or pharmaceutically acceptable salt or solvate thereof having a solubility of less than 0.1 mg/mL and a log P value greater than 2.5;
a permeation enhancer comprising at least one selected from the group of counterions, a pH modifier, a surfactant with a hydrophilic lipophilic balance (HLB) greater than 10, a bile salt, a micelle, or a fatty acid;
1-10 wt. % matrix former; and
1-10 wt. % of a structure former;
freezing the dosed pharmaceutical formulation; and
freeze-drying the frozen pharmaceutical formulation to form the dosage form.

61. The method of claim 60, wherein the permeation enhancer comprises a surfactant with a hydrophilic lipophilic balance greater than 10.

62. The method of claim 61, wherein the pharmaceutical formulation comprises 0.01-5 wt. % the surfactant.

63. The method of claim 61, wherein the surfactant has a concentration of 0.1-30× its critical micellar concentrations (CMC) in the pharmaceutical formulation.

64. The method of claim 63, wherein the surfactant has a concentration of 0.5-3× CMC in the pharmaceutical formulation.

65. The method of claim 61, wherein the pharmaceutical formulation has a molar ratio of API to the surfactant of 5:1 to 1:5.

66. The method of claim 60, wherein the permeation enhancer comprises a bile salt.

67. The method of claim 66, wherein the pharmaceutical formulation comprises 0.25-5 wt. % bile salt.

68. The method of claim 66, wherein the bile salt has a concentration of 0.5-5× CMC in the pharmaceutical formulation.

69. The method of claim 66, wherein the pharmaceutical formulation has a molar ratio of API to the bile salt of 15:1 to 1:15.

70. The method of claim 69, wherein the molar ratio is 5:1 to 1:5.

71. The method of claim 60, wherein the permeation enhancer comprises a fatty acid.

72. The method of claim 71, wherein the pharmaceutical formulation comprises 0.03-0.15 wt. % fatty acid.

73. The method of claim 71, wherein the pharmaceutical formulation has a molar ratio of API to fatty acid of 3:1 to 1:3.

74. The method of claim 60, wherein the permeation enhancer comprises counterions.

75. The method of claim 74, wherein the pharmaceutical formulation comprises 0.025-5 wt. % counterions.

76. The method of claim 74, wherein the pharmaceutical formulation has a molar ratio of API to counterions of 6:1 to 1:6.

77. The method of claim 76, wherein the molar ratio is 3:1 to 1:3.

78. The method of claim 74, wherein the pharmaceutical formulation comprises a pH modifier.

79. The method of claim 60, wherein the pharmaceutical formulation comprises 0.1-5 wt. % the BCS Class II API or pharmaceutically acceptable salt or solvate thereof.

80-126. (canceled)
